SEQUENCE LISTING (1 sequence)

SEQ ID NO: 1 - protein, 34 aa, synthetic construct
  DISULFID 2..17: note = Disulfide Cys2-Cys17
  DISULFID 9..23: note = Disulfide Cys9-Cys23
  DISULFID 16..30: note = Disulfied Cys16-Cys30
  MOD_RES 34: note = C-terminal amidation of Phe34
GCLEFWWKCNPNDDKCCRPKLKCSKLFKLCNFSF

(19) United States
(12) Patent Application Publication
(10) Pub. No.: US 2025/0352615 A1
(43) Pub. Date: Nov. 20, 2025

(54) COMPOSITIONS AND METHODS FOR THE MODULATION OF PIEZO1 AND TRPV4

(71) Applicant: Duke University, Durham, NC (US)

(72) Inventors: Rodger Liddle, Durham, NC (US); Joelle J. Romac, Durham, NC (US); Sandip Swain, Durham, NC (US)

(21) Appl. No.: 18/710,673

(22) PCT Filed: Nov. 14, 2022
(86) PCT No.: PCT/US22/79849

§ 371 Date: May 16, 2024

Related U.S. Application Data
(60) Provisional application No. 63/279,746, filed on Nov. 16, 2021.

(51) Int. Cl. A61K 38/17; A61K 31/121; A61K 31/18; A61K 31/353; A61K 31/4709; A61K 31/495; A61K 31/497; A61K 31/5377; A61K 33/24; A61P 35/00; A61P 35/04

(52) U.S. Cl. CPC A61K 38/1767; A61K 31/121; A61K 31/18; A61K 31/353; A61K 31/4709; A61K 31/495; A61K 31/497; A61K 31/5377; A61K 33/24; A61P 35/00; A61P 35/04

(57) ABSTRACT

Described herein are methods and compositions for treating or reducing the likelihood of a subject developing a pancreatic disease or disorder or keloids. In one embodiment, the method comprises administering a therapeutically effective amount of one or more of a Piezol antagonist, a PLA2 antagonist, a TRPV4 antagonist, or combinations thereof.

### CROSS-REFERENCE TO RELATED APPLICATIONS

This patent application is the U.S. national stage entry under 35 U.S.C. § 371 of International Application Number PCT/US2022/079849, filed Nov. 15, 2022, which claims priority to U.S. Provisional Patent Application No. 63/279,746, filed Nov. 16, 2021, the entire contents of each of which are hereby incorporated by reference.

### FEDERALLY SPONSORED RESEARCH

This invention was made with government support under grant number R01 DK120555 awarded by the National Institutes of Health. The government has certain rights in the invention.

### REFERENCE TO SEQUENCE LISTING

This application is filed with a Computer Readable Form of a Sequence Listing in accord with 37 C.F.R. § 1.821(c). The text file submitted in the USPTO Patent Center, “028193-0008-US02_sequence_listing_28 Oct. 2024_ST25.txt,” was created on Oct. 28, 2024, contains 1 sequence, has a file size of 4.00 kilobytes (4,096 bytes), and is incorporated by reference in its entirety into the specification.

### TECHNICAL FIELD

Described herein are methods and compositions for treating or reducing the likelihood of a subject developing a pancreatic disease or disorder or keloids. In one embodiment, the method comprises administering a therapeutically effective amount of one or more of a Piezo1 antagonist, a PLA2 antagonist, a TRPV4 antagonist, or combinations thereof.

### BACKGROUND

Disorders of the pancreas, such as pancreatic cancer and pancreatitis, are quite common. For example, pancreatitis is a severe, painful, and debilitating disease for which there is no specific treatment. Over 200,000 patients are hospitalized in the United States each year with pancreatitis and severe acute pancreatitis is associated with a ˜20% mortality rate. Treatment of pancreatitis has proven difficult once the disease has been initiated. In the case of cancer, in 2019, about 57,000 Americans were diagnosed with pancreatic cancer. Pancreatic cancer is slightly more common in men than in women, usually occurring after age 45. Pancreatic cancer's tendency to spread silently before diagnosis makes it one of the deadliest cancer diagnoses, with more than 45,000 people expected to die of the disease in 2019.

Stellate cells in the pancreas and other tissues are intimately associated with pancreatic disease, such as cancer, and the disease microenvironment. Stimulation can convert stellate cell from a quiescent to an activated phenotype leading to the production of fibrogenic and inflammatory proteins. In the pancreas, fibrogenic proteins contribute to the disease microenvironment and inflammatory proteins enhance the disease state, such as increased tumor formation and growth. Modulation of these proteins may provide a mechanism for the treatment of these pancreatic diseases or disorders.

Keloid is overgrowth of granulation tissue at the site of a scar beyond the normal boundaries of healing and is composed primarily of collagen. Type Ill collagen predominates in early stages and type I collagen appears in later stages of keloid growth. Keloids are generally firm or rubbery lesions that often grow in a claw-like pattern. Although they appear as tumors, keloids are benign, non-malignant tissue and are non-contagious. Keloids usually develop at the site of skin injury or trauma such as surgery, skin piercings, scratches, burns, chickenpox scars, vaccination sites, or acne. As fibrotic tumors, keloids contain atypical fibroblasts and extracellular matrix that is composed of collagen, fibronectin, elastin, and proteoglycans. They are relatively acellular although fibroblasts can be seen throughout the lesions.

Injury to skin initiates a fibrotic response through the stimulation of myofibroblasts which secrete collagen. Myofibroblasts, characterized by expression of glial fibrillary acidic protein (GFAP) and α-smooth muscle actin expression, reside in close proximity to keratinocytes and other dermal elements in the skin. GFAP-expressing cells are present in both the epidermis and dermis. However, epidermal cells express a higher level of GFAP than dermal cells. Piezo1 and GFAP co-localized in the majority of cells. Collagen-producing stellate cells in the pancreas express GFAP and Piezo1 and respond to mechanical force. Prolonged stimulation induces these cells to produce collagen. High levels of Piezo1 and TRPV4 are expressed in human keloid scar and based on the discovery that Piezo1 mediated mechano-signaling pathways induced TRPV4 channel activation is required for abnormal collagen synthesis in pancreas, suggests that this phenomenon may operate in keloid where it would be responsible for high levels of collagen deposition and unusual skin growth.

What is needed are compositions and methods for the treatment or prophylaxis of pancreatic diseases or disorders or keloids by modulating Piezo1 and TRPV4.

### SUMMARY

One embodiment described herein is a method for treating a subject suffering from or reducing the likelihood of developing a pancreatic disease or disorder, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof. In one aspect, the Piezo1 antagonist comprises GsMTx-4. In another aspect, the TRPV4 antagonist comprises Ruthenium Red, RN-1734, HC-067047, RN-9893, GSK2798745, GSK2193874, or combinations thereof. In another aspect, the PLA2 antagonist comprises YM26734, AACOCF₃, or a combination thereof. In another aspect, the pancreatic disease or disorder comprises pancreatitis, pancreatic fibrosis, pancreatic cancer, metastatic pancreatic cancer, or combinations thereof. In another aspect, the pancreatic cancer or the metastatic pancreatic cancer comprises pancreatic ductal adenocarcinoma (PDAC). In another aspect, the method of further comprises administering one or more additional therapeutic agents to the subject. In another aspect, the one or more additional therapeutic agents is selected from chemotherapeutic agents, anticancer agents, anti-inflammatory agents, antibiotics, steroids, or combinations thereof. In another aspect, the one or more additional therapeutic agents is administered to the subject before administration of the pharmaceutical composition comprising the inhibitory molecule. In another aspect, the one or more additional therapeutic agents is administered to the subject concurrently with administration of the pharmaceutical composition comprising the inhibitory molecule. In another aspect, the one or more additional therapeutic agents is administered to the subject after administration of the pharmaceutical composition comprising the inhibitory molecule.

Another embodiment described herein is a method for treating or reducing the likelihood of pancreatic stellate cell activation and/or activation of a fibrinogenic or inflammatory phenotype in a subject, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

Another embodiment described herein is a method for treating or reducing the likelihood of a subject developing keloids, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof. In one aspect, the Piezo1 antagonist comprises GsMTx-4. In another aspect, the TRPV4 antagonist comprises Ruthenium Red, RN-1734, HC-067047, RN-9893, GSK2798745, GSK2193874, or combinations thereof. In another aspect, the PLA2 antagonist comprises YM26734, AACOCF₃, or a combination thereof. In another aspect, the method of further comprises administering one or more additional therapeutic agents to the subject. In another aspect, the one or more additional therapeutic agents is selected from anti-inflammatory agents, antibiotics, steroids, or combinations thereof. In another aspect, the one or more additional therapeutic agents is administered to the subject before administration of the pharmaceutical composition comprising the inhibitory molecule. In another aspect, the one or more additional therapeutic agents is administered to the subject concurrently with administration of the pharmaceutical composition comprising the inhibitory molecule. In another aspect, the one or more additional therapeutic agents is administered to the subject after administration of the pharmaceutical composition comprising the inhibitory molecule.

Another embodiment described herein is a method for treating or reducing the likelihood of fibroblast activation and/or activation of a fibrinogenic or inflammatory phenotype in a subject, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

Another embodiment described herein is a pharmaceutical composition for treating a subject suffering from, or reducing the likelihood of developing, a pancreatic disease or disorder or keloid, the pharmaceutical composition comprises one or more inhibitory molecules comprising a Piezo1 antagonist, a PLA2 antagonist, a TRPV4 antagonist, or combinations thereof. In one aspect, the pharmaceutical composition further comprises one or more pharmaceutically acceptable carriers or excipients. In another aspect, the Piezo1 antagonist comprises GsMTx-4. In another aspect, the TRPV4 antagonist comprises Ruthenium Red, RN-1734, HC-067047, RN-9893, GSK2798745, GSK2193874, or combinations thereof. In another aspect, the PLA2 antagonist comprises YM26734, AACOCF₃, or a combination thereof. In another aspect, the pharmaceutical composition further comprises one or more additional therapeutic agents selected from chemotherapeutic agents, anticancer agents, anti-inflammatory agents, antibiotics, steroids, or combinations thereof.

Another embodiment described herein is the use of the pharmaceutical compositions described herein as medicaments for treating a subject suffering from, or reducing the likelihood of developing, a pancreatic disease or disorder or keloid.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-G show that Piezo1 activation increases [Ca²⁺]ᵢ and induces cell death in pancreatic acini. FIG. 1A shows live-cell imaging of pancreatic acini loaded with calcium 6-QF. Pancreatic acini were incubated with Yoda1 (25 μM) in the presence (2 mM) or absence (0 mM) of extracellular calcium. Yoda1 was added at the time indicated by the arrow. FIG. 1B shows a comparison of the Yoda1-induced peak [Ca²⁺]ᵢ expressed as the ratio of peak intensity (Fₘₐₓ)/baseline intensity (F₀) from 32 to 41 cells. FIG. 1C shows the effects of Yoda1 (50 μM) on [Ca²⁺]ᵢ in the absence or presence of the calcium chelator BAPTA-AM (20 μM). FIG. 1D shows statistical analysis of peak (Fₘₐₓ/F₀) from 25 to 30 cells in which acinar cells were preincubated 30 minutes with BAPTA-AM (20 μM) before Yoda1 application. (Fₘₐₓ/F₀) was calculated from the time periods 1 to 2 minutes and 5 to 6 minutes to assess the initial transient and sustained [Ca²⁺]ᵢ levels, respectively. FIG. 1E shows the effects of CCK (1 nM) on LDH release from isolated pancreatic acini from WT and Piezo1^{aci}-KO mice from 4 to 6 experiments. FIG. 1F shows the effects of Yoda1 (50 μM) on LDH release from acinar cells with and without preincubation of BAPTA-AM for 30 minutes, shown from 3 to 5 experiments. FIG. 1G shows brightfield images of pancreatic acini at different time points in the presence of Yoda1 (50 μM). Images represent a plane (5 μm thick) from a Z-stack of captured images to visualize the changes in cell morphology and granule movement. Images were captured with a ×100 oil objective. Statistical analyses were performed using Student's t test (FIGS. 1B and 1D) and 1-way ANOVA with Tukey's multiple comparison test (FIGS. 1E and 1F). *P≤0.05; **P≤0.01; ***P≤0.001, ****P≤0.0001. Data are shown as mean±SEM. Scale bar: 10 μm.

FIG. 2A-B show the effect of extracellular and intracellular Ca²⁺ ion on CCK-induced [Ca²⁺]ᵢ. FIG. 2A shows representative traces of CCK-induced [Ca²⁺]ᵢ elevation with and without pre-incubation with BAPTA-AM. FIG. 2B shows isolated mouse pancreatic acini that were incubated with CCK (1 nM) in the presence of bath calcium (2 mM) or absence of extracellular calcium, and some cells were pre-incubated 30 minutes with BAPTA-AM (20 μM) before CCK application. The CCK-induced peak [Ca²⁺]ᵢ is expressed as the ratio of peak intensity (Fₘₐₓ)/baseline intensity (F₀) from 35-37 cells. Statistical analyses were performed using Student's t-test. **P≤0.01; ****P≤0.0001. Results are expressed as the mean±SEM.

FIG. 3A-E show that the CRAC blocker CM4620 inhibits CCK- but not Yoda1-induced elevations in [Ca²⁺]ᵢ in pancreatic acini. FIG. 3A shows representative traces showing the [Ca²⁺]ᵢ elevation following incubation with CCK (100 μM) or CCK (100 μM)+CM4620. Mouse pancreatic acini were treated with CCK (100 μM) in the absence or presence of CM4620. CM4620 was added 1 hr before CCK. FIG. 3B shows peak [Ca²⁺]ᵢ responses shown from acini treated with CCK (100 μM) or CCK (100 μM)+CM4620, (n=4 experiments with 47-68 cells). FIG. 3C shows peak [Ca²⁺]ᵢ responses shown from acini treated with CCK (1 nM) or CCK (1 nM)+CM4620, (n=3 experiments with 34-36 cells). FIG. 3D shows a representative experiment showing the effects of CM4620 (5 μM) on Yoda1 (25 μM)-induced [Ca²⁺]ᵢ in pancreatic acini. Acini were pre-incubated with CM4620 or vehicle for 1 hr. FIG. 3E shows the effects of CM4620 on Yoda1-induced [Ca²⁺]ᵢ shown at two time periods (1-2 minutes and 4-5 minutes) (n=3 experiments with 30-32 cells). Statistical analyses were performed using Student's t-test. ***P≤0.001; ****P≤0.0001. Values represent the mean±SEM.

FIG. 4A-H show that activation of Piezo1 does not alter CCK sensitivity in pancreatic acini. FIG. 4A-C show CCK-stimulated live cell calcium imaging in pancreatic acini from wild type (WT) and Piezo1^{aci} KO mice loaded with Calcium 6-QF dye. FIG. 4A shows traces that represent the live cell calcium imaging with CCK (1 nM) from three experiments. FIG. 4B shows the maximum peak [Ca²⁺]ᵢ expressed as the ratio of maximum peak intensity (Fₘₐₓ)/baseline intensity (F₀), and FIG. 4C shows the sustained calcium [Ca²⁺]ᵢ rise expressed as the ratio of peak [Ca²⁺]ᵢ at 9 minutes (F₉)/base line intensity (F₀) from 30-32 cells. FIG. 4D shows that CCK responsivity is not altered in Piezo1^{aci}-KO mice. The maximum CCK (20 μM)-stimulated [Ca²⁺]ᵢ elevation is shown in acini from wild type or Piezo1^{aci} KO mice (from 33 cells). FIG. 4E shows the effects of acinar cell pushing with a micropipette and physiological concentrations of CCK (20 μM) on live acinar cell [Ca²⁺]ᵢ. A representative image from 5 experiments is shown. FIG. 4F shows peak [Ca²⁺]ᵢ levels from 5 experiments with a total of 18-19 cells. FIG. 4G-H show the viability of pancreatic acini from WT mice measured following stimulation with CCK (1 nM), Yoda1 (50 μM), and CCK (1 nM)+Yoda1 (50 μM) using Propidium Iodide and Calcein AM dyes. Dead cells were marked by Propidium Iodide uptake (red) and live cells were labeled with Calcein AM (green) (scale bar=100 μm). Quantitative values for pancreatic acinar cell viability from 3 experiments are shown in FIG. 4H. Statistical analyses were performed using Student's t-test (FIGS. 4B-D and 4F) and one-way ANOVA with the Tukey's multiple comparison (FIG. 4H). *P≤0.05, **P≤0.01; ****P≤0.0001. Results are expressed as the mean±SEM.

FIG. 5A-1 show that Piezo1 activation induces mitochondrial depolarization and trypsinogen activation in pancreatic acini. FIG. 5A shows brightfield and fluorescence images of TMRE-loaded pancreatic acini 0 and 12 minutes after Yoda1 (50 μM) application. FIG. 5B shows traces of live-cell TMRE fluorescence intensity of single acinar cells over time following the administration of Yoda1 (arrow). F_{D} is the decrease in TMRE fluorescence, and F₀ is the baseline TMRE intensity. Acini from WT or Piezo1^{ad}i-KO mice were treated with the mitochondrial uncoupler FCCP (10 μM), Yoda1 (50 μM), Yoda1+GsMTx4 (5 μM), or CCK (10 nM). A representative tracing from 3 experiments is shown. FIG. 5C shows the mean decrease in TMRE intensity from FIG. 5B (n=3-5 independent experiments with 15-20 cells in each experiment). FIG. 5D shows a decrease in TMRE intensity of pancreatic acini in response to CCK (10 nM) from 60 cells over 20 minutes. FIG. 5E shows a decrease in fluorescence intensity of TMRE in pancreatic acinar cells upon Yoda1 application with or without preincubation of BAPTA-AM (20 μM) for 20 minutes from 29-45 cells. FIG. 5F shows live-cell imaging of intracellular trypsin activation with CCK (10 nM) and Yoda1 (50 μM) treatments. Scale bar: 10 μm. FIG. 5G shows a time course of Yoda1-induced trypsin activation for 3 experiments. FIG. 5H shows peak BZiPAR fluorescence that was measured in acini from WT mice treated with Yoda1 in the absence or presence of GsMTx4 (5 μM) or in acini from Piezo1^{aci}-KO mice (n=3-5 independent experiments with 16-31 cells). FIG. 51 shows that pancreatic acini from Piezo1^{aci}-KO mice had a trypsin activation response to CCK (10 nM) similar to that of WT mice (n=3 from 16-20 cells). Statistical analyses were performed using Student's t test (FIG. 5D) and 1-way ANOVA (FIGS. 5C, 5E, and 5H-1). Data are shown as mean±SEM. **P≤0.01; ***P≤0.001; ****P≤0.0001.

FIG. 6A-J show that mechanical pushing and fluid shear stress increase [Ca²⁺]ᵢ in pancreatic acini. FIG. 6A shows brightfield and live-cell imaging of pancreatic acini loaded with calcium 6-QF at time 0 and at time of maximum fluorescence 1:40 (min:s) after mechanical pushing. Scale bars: 10 μm. FIG. 6B shows representative [Ca²⁺]ᵢ fluorescence tracings from single acinar cells in an acinus during the course of mechanical pushing with a blunt pipette. FIG. 6C shows peak [Ca²⁺]ᵢ levels following mechanical pushing of acini from WT or Piezo1^{aci}-KO mice. FIG. 6D shows that blunt pushing with a micropipette increased [Ca²⁺]ᵢ fluorescence only in the presence of extracellular Ca²⁺. Results represent data from 25-39 cells in 3 independent experiments. FIG. 6E shows representative traces for relative fluorescence intensity (ΔF/F₀) of calcium 6-QF-loaded cells in response to applied shear stress at the forces shown in the graph. The duration of fluid flow shear stress was 30 seconds, as indicated by the orange bar. FIG. 6F shows the average peak [Ca²⁺]ᵢ intensity of Fₘₐₓ/F₀ for 41-54 cells (n=3-4 independent experiments). FIG. 6G-H show the relative fluorescence intensity (ΔF/F₀) and average peak [Ca²⁺]ᵢ intensity of pancreatic acini in response to 12 dyne/cm² shear stress for 1 or 5 seconds. Data in FIG. 6H are averaged from 14-41 cells. FIG. 6I shows representative traces of relative fluorescence intensity (ΔF/F₀) from calcium 6-QF-loaded WT and Piezo1-KO acinar cells before and after applying 12 dyne/cm² shear stress for 30 seconds. FIG. 6J shows the average peak intensity of Fₘₐₓ/F₀ from the experiment depicted in FIG. 6I. Data represent a total of 53-67 cells and 3 independent experiments. Statistical analyses were performed using Student's t test (FIGS. 6D, 6H, and 6J) and 1-way ANOVA with Tukey's multiple comparison (FIGS. 6C and 6F). Data are shown as mean±SEM. **P≤0.01; ***P≤0.001; ****P≤0.0001.

FIG. 7A-D show that fluid shear stress induces mitochondrial depolarization and trypsinogen activation in pancreatic acini. FIG. 7A shows pancreatic acini were subjected to fluid shear force of 12 dyne/cm², and TMRE dye fluorescence intensity of pancreatic acinar cells from WT and Piezo1^{aci}-KO mice were measured over 15 minutes. The duration of shear stress for 30 seconds is marked by the orange bar. F_{D} represents the decrease in TMRE fluorescence over time, and F₀ represents the base line TMRE intensity before fluid shear stress. FIG. 7B shows the mean average decrease in fluorescence intensity of TMRE over 15 minutes. Flow is the lowest TMRE fluorescent intensity after fluid shear stress during imaging. The total number of acinar cells examined were as follows: control (without fluid shear stress)=56; fluid shear stress on WT acini=50; and fluid shear stress on Piezo1^{aci}-KO acini=91 and WT acini=67 (n=3 independent experiments). FIG. 7C shows traces that represent live-cell trypsin activity upon fluid shear stress of 12 dyne/cm² for 30 seconds in acini from WT or Piezo1^{aci}-KO mice (n=3 experiments). FIG. 7D shows peak fluorescence intensity of BZiPAR over 50 minutes from 3 experiments; total number of acinar cells were as follows: WT=28 and Piezo1^{aci}-KO=36. Data are shown as the mean±SEM. ****P≤0.0001. Statistical analyses were performed using Student's t test (FIG. 7B) and 1-way ANOVA with Tukey's multiple comparison (FIG. 7D).

FIG. 8A-B show mechanically-induced mitochondrial depolarization and trypsinogen activation. FIG. 8A shows the change in TMRE intensity of pancreatic acini upon 5 μm mechanical pushing for 1 second. Flow is the lowest fluorescence intensity over 12 minutes after pushing and F₀ is the basal fluorescence intensity before pushing. 48-53 cells were used in the analysis from 3 independent experiments. FIG. 8B shows the increase in BZiPAR intensity of pancreatic acini upon 5 μm mechanical pushing for 1 second. Peak BZiPAR fluorescence intensity over 50 minutes from 20-26 cells. Statistical analyses were performed using Student's t-test.

FIG. 9A-C show immunostaining of TRPV4 in mouse and human pancreatic acini and Piezo1 in human pancreatic acini. FIG. 9A (left panel) shows mouse pancreatic acini immunostained with TRPV4 antibody (red), and nuclei were identified with Nunc blue. FIG. 9A (right panel) shows pancreatic acini immunostained with TRPV4 antibody pre-absorbed with immunogenic peptide to eliminate specific, saturable TRPV4 staining. FIG. 9B shows human pancreatic acini immunostained with Piezo1 antibody (green), and nuclei were identified with Nunc blue. FIG. 9C shows human pancreatic acini immunostained with TRPV4 antibody (red), and nuclei were identified with Nunc blue. Scale bar=10 μm.

FIG. 10A-L show that TRPV4 is expressed in pancreatic acini, and Piezo1 induces TRPV4 activation. FIG. 10A shows mRNA (fold expression) of TRPV4 and Piezo1 in pancreatic acini relative to the housekeeping gene actb (n=3-5 experiments). FIG. 10B shows immunostaining of mouse pancreatic acini with a TRPV4 antibody (red). Nuclei (blue) were stained with Nunc blue. Scale bar: 10 μm. FIG. 10C-D show the effects of the TRPV4 agonist GSK101 (50 nM) and GSK101+the TRPV4 antagonist HC067 (100 nM) or RN1734 (30 μM) on [Ca²⁺]ᵢ. FIG. 10C shows a representative experiment showing the relative fluorescence intensity (ΔF/F₀) of calcium dye over time and FIG. 10D shows the average peak [Ca²⁺]ᵢ intensity of pancreatic acini from 43-63 cells. FIG. 10E-F show that arachidonic acid (20 μM) and 5′,6′-EET (5 μM) increased [Ca²⁺]ᵢ. The effects of 5′,6′-EET (5 μM) were blocked by HC067 (100 nM). FIG. 10E shows the relative fluorescence intensity of calcium dye and FIG. 10F shows the average peak [Ca²⁺]ᵢ intensity of pancreatic acini from 40-54 cells. FIG. 10G-J show that the TRPV4 antagonist HC067 (1 μM) blocked the effects of shear stress (12 dyne/cm²) and Yoda1 (25 μM) on the sustained [Ca²⁺]ᵢ responses. FIGS. 10G and 101 show representative tracings of the relative fluorescence intensity of calcium dye over time with the different stimuli. FIGS. 10H and 10J show the average peak [Ca²⁺]ᵢ responses calculated from 35-86 cells. FIG. 10K-L show that shear stress- and Yoda-induced sustained increases in [Ca²⁺]ᵢ were not seen in pancreatic acini isolated from TRPV4-KO mice. FIG. 10K shows representative traces demonstrating the effects of shear stress or Yoda1 on [Ca²⁺]ᵢ and FIG. 10L shows the average peak [Ca²⁺]ᵢ intensity at different time intervals (from 45-47 cells). Statistical analyses were performed using Student's t test. Data are shown as the mean±SEM. *P≤0.05; ***P≤0.001; ***P≤0.0001.

FIG. 11A-E show that the Piezo1 agonist, Yoda1, induces activation of PLA2 activity. FIG. 11A shows brightfield and fluorescence images of BODIPY FL C₁₁-PC loaded pancreatic acini at time 0 and 3:30 minutes after Yoda1 (25 μM) application. Scale bar: 10 μm. FIG. 11B shows traces representing the live-cell PLA2 activity upon Yoda1 (25 M) application from 4 experiments. Representative tracings of acini from WT and Piezo1^{aci}-KO mice are shown. The peak fluorescence intensity was calculated from the elapsed time at 5-6 minutes (blue bar). FIG. 11C shows peak fluorescence intensity of BODIPY FL C₁₁-PC dye measured at an elapsed time of 5-6 minutes from 40-51 cells. FIG. 11D-E show traces and graphs showing the effects of Yoda1 (25 μM) on [Ca²⁺]ᵢ in pancreatic acini with or without treatment with the cytoplasmic PLA2 blocker AACOCF3 (30 μM) and secretory PLA2 blocker YM26734 (10 μM). YM26734 and AACOCF3 were preincubated 10 minutes before application of Yoda1. The transient calcium peaks were measured from signals obtained between 1-3 minutes (yellow bar), and sustained calcium peaks were measured from signals from 5.5-6.5 minutes (pink bar). Data represent the averages of 36-58 cells. Values were expressed as the mean±SEM, and mean differences between multiple groups were analyzed by 1-way ANOVA with Tukey's multiple comparison. *P≤0.05; *** P≤0.001; ****P≤0.0001. Scale bar: 10 μm.

FIG. 12 shows that Protein kinase A and C blockers did not significantly affect the Yoda1-induced sustained calcium elevation. Yoda1-induced peak calcium rises with or without protein kinase A blocker, KT5720 (5 μM), or protein kinase C blocker, GF109203X (20 μM), is shown. The transient calcium peaks were measured from signals between 1-3 minutes and sustained calcium peaks were measured from signals between 5-6-minute intervals. A total of 30 cells were used in the analysis. Statistical analyses were performed using Student's t-test.

FIG. 13A-L show that TRPV4-KO mice are protected against Yoda1- and pancreatic pressure-induced pancreatitis. FIG. 13A shows photographs of the surgical approach used for injection of Yoda1 into the pancreatic duct. Methylene blue dye was mixed with the injected solution to aid with pancreatic duct visualization. A total of 50 μL was injected over 10 minutes. The amount of Yoda1 injected into the pancreatic duct was 0.4 mg/kg. The boundary of the mouse pancreas is marked with yellow before and after Yoda1 infusion. After injection, methylene blue in the solution made the pancreas blue in color. Pancreatitis parameters included edema (FIG. 13B), serum amylase (FIG. 13C), tissue MPO (FIG. 13D), and pancreatic histology score (FIG. 13E) in vehicle- and Yoda1-treated WT and TRPV4-KO mice (n=3-5). FIG. 13F shows representative H&E-stained images of the midregion of pancreas. Scale bar: 100 μm. FIG. 13G shows a photograph of the partial PDL procedure. Head and tail regions of pancreas are outlined in yellow and blue, respectively. PDL-induced pancreatitis parameters included edema (FIG. 13H), serum amylase (FIG. 13I), pancreatic MPO (FIG. 13J), and pancreas histology scores (FIG. 13K) of the sham and PDL WT and TRPV4-KO mice (n=5-7). FIG. 13L shows representative H&E images of the midregion of the pancreas. Scale bar: 100 μm. Statistical analyses were performed using 1-way ANOVA with Tukey's multiple comparison. Data are shown as mean±SEM. *P≤0.05; **P≤0.01; ***P≤0.001; ****P≤0.0001.

FIG. 14A-G show that caerulein-induced pancreatitis is independent of TRPV4 channel activation. FIG. 14A shows live-cell imaging of pancreatic acini loaded with Calcium 6-QF. Pancreatic acini from wild type mice were incubated with CCK (1 nM) with or without HC067 (1 μM). FIG. 14B shows the maximum CCK-induced peak [Ca²⁺]ᵢ expressed as the ratio of maximum peak intensity (Fₘₐₓ)/baseline intensity (F₀), and FIG. 14C shows the sustained [Ca²⁺]ᵢ expressed as the ratio of peak [Ca²⁺]ᵢ at 9 minutes (F₉)/baseline intensity (F₀) from 33 cells. FIG. 14D-E show caerulein-induced pancreatitis in wild type and TRPV4 KO mice. The pancreatitis parameters presented are edema (FIG. 14D), serum amylase (FIG. 14E), tissue MPO (FIG. 14F), and pancreatic histology score (FIG. 14G) in vehicle- and CCK-treated wild type (WT) and TRPV4 KO mice (n=3-5). FIG. 14H shows representative H&E-stained images of the midregion of pancreas (scale bar=100 μm). Statistical analyses were performed using Student's t-test (FIG. 14B-C) and one-way ANOVA with the Tukey's multiple comparison (FIG. 14D-G). Values represent the mean±SEM; **P≤0.01, ***P≤0.001.

FIG. 15A-K show that Piezo1^{GFAP}-KO mice were protected from pancreatic duct ligation-induced fibrosis. FIG. 15A shows a photograph of pancreatic duct ligation (PDL) at the tail region of the pancreas. The head and tail regions of the pancreas are outlined in white and yellow, respectively. FIG. 15B shows pressure in the tail region of the pancreas before and 5 minutes after pancreatic duct ligation. FIG. 15C shows immunostaining of GFAP and Piezo1 in mouse PSCs. FIG. 15D-G show that eight days after PDL, chronic pancreatitis and fibrosis parameters included (FIG. 15D) tail region H&E staining, (FIG. 15E) tail region H&E score, (FIG. 15F) tail region Masson's trichrome staining, and (FIG. 15G) tail Masson's trichrome area of WT, Piezo1^{aci}-KO, and Piezo1^{GFAP}-KO mice (n=3-7). FIG. 15H-K show that thirty days after PDL, chronic pancreatitis and fibrosis parameters included tail region (FIG. 15H) H&E staining, (FIG. 15I) H&E score, (FIG. 15J) tail region Masson's trichrome staining, and (FIG. 15K) Masson's trichrome area of WT and Piezo1^{GFAP}-KO mice (n=3-6). Statistical analyses were calculated using 2-tailed Student's t test for 2 groups, and multiple groups were analyzed by 1-way ANOVA. **P≤0.01, ***P≤0.001; ****P≤0.0001. Scale bar: 100 μm.

FIG. 16 shows that pancreatic duct ligation at the tail region did not affect the head of the pancreas. Representative H&E-stained images of the pancreatic head region of wildtype (WT) and Piezo1^{GFAP}-KO mice are shown 8 days after PDL (n=3-7).

FIG. 17A-C show Piezo1 deletion in GFAP-expressing PSCs. FIG. 17A shows images showing expression of GFAP (green) in mouse PSCs after 3 days in culture. Cell nuclei were stained with Nunc blue. FIG. 17B shows PSCs expressing Cre protein from the mouse line B6.Cg-Tg (GFAP-cre/ERT2); Piezo1^{fl/fl} after tamoxifen injection (referred to as Piezo1^{GFAP}-KO mice) confirm deletion of Piezo1 in PSCs. Scale bar: 20 μm. All cells (Nunc blue), cells expressing Cre protein, and cells expressing GFAP appear as merged images. FIG. 17C shows quiescent PSCs containing perinuclear fat droplets (stained with Bodipy™ 593/503) cultured on a Matrigel coated plate. Scale bar: 10 μm.

FIG. 18A-Q show functional expression of Piezo1 in pancreatic stellate cells. FIG. 18A-B show a tracing and graph illustrating the dose-dependent effects of the Piezo1 agonist, Yoda1, on [Ca²⁺]ᵢ in mouse PSCs (from 33-35 cells). The arrow in FIG. 18A represents the time of Yoda1 application. FIG. 18C-D show the effects of Yoda1 (25 μM) on [Ca²⁺]ᵢ in PSCs from WT and Piezo1^{GFAP}-KO mice (from 40 cells). FIG. 18E shows that the effects of Yoda1 on the rise in [Ca²⁺]ᵢ was blocked by the Piezo1 antagonist GsMTx4 (5 μM) (from 16-21 cells). FIG. 18F-G show that Yoda1 increased [Ca²⁺]ᵢ in the presence and absence of external calcium (from 13 cells). FIG. 18H shows immunostaining of GFAP, nuclei (with Nunc blue), and Piezo1 in human PSCs. Merged images of GFAP (green) and Piezo1 (red) appear as yellow. FIGS. 18I-J show that the Yoda1-mediated [Ca²⁺]ᵢ rise in human PSCs was blocked with GsMTx4 (5 μM) (from 20-50 cells). FIG. 18K-L show a tracing and graph representing the effects of fluid shear stress (12 dyne/cm²) applied for 1 minute on [Ca²⁺]ᵢ with or without GsMTx4 (5 μM) (from 41-45 cells). FIG. 18M-O show representative tracings showing the effects of fluid shear stress applied at 4 dyne/cm² for 1 minute, 12 dyne/cm² for 5 seconds, and 12 dyne/cm² for 1 minute on [Ca²⁺]ᵢ in human PSCs. FIG. 18P-Q show statistical comparisons of the peak and sustained [Ca²⁺]ᵢ rise for the fluid shear stresses shown in FIG. 18M-O (from 30-45 cells). The sustained elevation in [Ca²⁺]ᵢ was measured at 8 minutes of imaging. Data are presented as mean±SEM. Statistical analyses were calculated using 2-tailed Student's t test for 2 groups, and multiple groups were analyzed by 1-way ANOVA. *P≤0.05; **P≤0.01, ***P≤0.001; ****P≤0.0001. Scale bar: 10 μm.

FIG. 19A-C show that high dose Yoda1 did not affect PSC viability and membrane integrity. FIG. 19A shows that mouse PSCs responded to ionomycin (1 μM) after Yoda1 (25 μM) treatment. FIG. 19B-C show that PSCs were treated with Yoda1 (25 μM) for 2 hr after which the viability of stellate cells was analyzed using the Live/Dead Cell Imaging Kit (Thermo Fisher Scientific, Catalog #R37601). Representative images show live (green) and dead (red) cells. FIG. 19C shows a graph representing viable cells with and without Yoda1 treatment (n=3 experiments).

FIG. 20A-C show that mechanical pushing increases [Ca²⁺]ᵢ in PSCs. FIG. 20A shows brightfield and live-cell images of mouse PSC at time 0 and at 1:40 (min) after mechanical pushing with a blunt tip glass pipette for 1 sec. FIG. 20B shows a representative [Ca²⁺]ᵢ profile from a single mouse PSC during the course of mechanical pushing. FIG. 20C shows a graph showing peak [Ca²⁺]ᵢ levels following mechanical pushing in PSCs with and without GsMTx4 (2.5 μM). **P<0.01, n=4-5.

FIG. 21A-K show that the Piezo1 agonist, Yoda1, induces stellate cell activation and fibrosis in vitro. FIG. 21A shows that fluorescent dye, Bodipy 493/503-stained fat droplets (green) are visible in human PSCs 24 hr after Yoda1 (25 μM) treatment. FIG. 21B shows a graph representing the Yoda1-induced loss of fat droplets (from 3 experiments; >100 cells analyzed for each experiment). FIG. 21C shows differential interference contrast (DIC) images of human PSCs with or without Yoda1 treatment (25 μM for 24 hr). FIG. 21D-E show the mean cell area and mean Feret's diameter (max) of human PSCs with and without Yoda1 (25 μM) (from 3 experiments; >20 cells analyzed in each experiment). FIG. 21F and 21I show representative images of fibronectin and collagen type I immunostaining in human PSCs 4 days after Yoda1 (25 μM) (from 3 experiments). FIGS. 21G-H and 21J-K show mRNA levels of fibronectin, collagen type 1, TGF-β1, and Piezo1 in human PSCs 24 hr after treatment with Yoda1 (25 μM) (3-5 experiments). Data are shown as mean±SEM. Scale bars: 10 μm. Statistical analyses were calculated using 2-tailed Student's t test for 2 groups, and multiple groups were analyzed by 1-way ANOVA. *P≤0.05; **P≤0.01, ****P≤0.0001.

FIG. 22A-F show that Yoda1 caused stellate cell activation and conversion to a fibroblast phenotype. FIG. 22A shows DIC images and Bodipy 493/503-stained fat droplets (green) in WT and Piezo1^{GFAP}-KO mouse PSCs without Yoda1 and 24 hr after Yoda1 (25 μM). The images were captured using MetaMorph 7.10.1 software from Molecular Devices. FIG. 22B shows Yoda1-induced loss of fat droplets (from 3 experiments; >100 cells per experiment). FIGS. 22C and 22E show representative images of fibronectin and collagen type I immunostaining in mouse PSCs 4 days after Yoda1 (25 μM) (representative of 3 experiments). FIGS. 22D and 22F show quantification of the fibronectin and collagen type I intensity (arbitrary units) calculated from data shown in FIGS. 22C and 22E. Data are shown as mean±SEM. Scale bars: 10 μm. Statistical analyses were calculated for multiple groups by 1-way ANOVA. **P≤0.01, ***P≤0.001.

FIG. 23A-F show fluid shear stress-induced stellate cell activation and fibrosis in vitro. FIG. 23A shows the fluid shear stress-(12 dyne/cm² for 10 minutes) induced loss of fat droplets (green) stained with Bodipy 493/503 in human PSCs with and without GsMTx4 (5 μM). Images were taken 24 hr after shear stress. FIG. 23B shows quantification of reduction in fat droplets 24 hr after shear stress (from 3 experiments; >50 cells per experiment). FIGS. 23C and 23E show representative images of fibronectin and collagen type I immunostaining in human PSCs 3 days after the last application of shear stress. Shear stress (25 dyne/cm² for 10 minutes) was applied twice at an interval of 24 hr. FIGS. 23D and 23F show quantification of the fibronectin and collagen type I intensity calculated from data shown in FIGS. 23C and 23E. Data represent the mean±SEM. Statistical analyses were calculated using 1-way ANOVA. **P≤0.01, ***P≤0.001; ****P≤0.0001. Scale bar: 10 μm.

FIG. 24A-O show that Piezo1 mediates TRPV4 channel opening in pancreatic stellate cells. FIG. 24A shows immunostaining of GFAP and TRPV4 in human PSCs. FIG. 24B shows TRPV4 agonist GSK101 (100 nM) effects on [Ca²⁺]ᵢ in human PSCs with and without the TRPV4 blocker HC067 (1 μM). FIG. 24C shows that the GSK101 (100 nM) effects on [Ca²⁺]ᵢ in PSCs from 3 biological samples were blocked with the TRPV4 antagonist HC067 (1 μM) (from 18-37 cells). FIG. 24D shows immunostaining of GFAP and TRPV4 in mouse PSCs. FIG. 24E-F show traces and graphs representing the effects of the TRPV4 agonist GSK101 (100 nM) on [Ca²⁺]ᵢ in mouse PSCs with and without the TRPV4 blocker HC067 (1 μM) (from 18 cells). FIG. 24G-I show the effects of Yoda1 (25 μM) on [Ca²⁺]ᵢ in PSCs from WT and TRPV4-KO mice. FIG. 24I shows statistical analyses of peak and sustained [Ca²⁺]ᵢ elevation (from 24-32 cells). The sustained [Ca²⁺]ᵢ elevation was measured at 6 minutes after Yoda1. FIGS. 24J and 24K show the effects of phospholipase A2 blockers AACOCF3 (30 μM) and YM26734 (10 μM) on Yoda1-induced (25 μM) [Ca²⁺]ᵢ in PSCs (from 24-26 cells). The sustained calcium rise was measured at 8 minutes after Yoda1 application. FIG. 24L-N show fluid shear stress (12 dyne/cm²) applied for 1 minute in PSCs from WT and TRPV4-KO mice and TRPV4-KO mice with GsMTx4 (5 μM). In FIGS. 24G-H, and 24L-N, each colored line represents the response of a single cell. FIG. 24O shows quantification of peak [Ca²⁺]ᵢ following shear stress (12 dyne/cm²) for 1 minute in TRPV4-KO PSCs with and without GsMTx4 (from 24 cells). Statistical analyses were calculated using 2-tailed Student's t test. *P≤0.05 and ****P≤0.0001. Scale bar: 10 μm.

FIG. 25A-M show that TRPV4-KO mice were protected from pancreatic duct ligation-induced fibrosis. FIGS. 25A and 25D show DIC and Bodipy 493/503-stained images of PSCs from TRPV4-KO mice 24 hr after Yoda1 (25 μM). FIG. 25B-C show the mean cell area and Feret's diameter (max) of PSCs 24 hours after Yoda1 (25 μM) (from 3 experiments with 20 cells each). FIG. 25E shows the loss of fat droplets in PSCs following Yoda1 (25 μM) (from 3 experiments and >100 cells). FIG. 25F-G show the quantification of collagen type I and fibronectin immunostaining in PSCs from TRPV4-KO mice 4 days after Yoda1 (25 μM). FIG. 25H-I show representative images of collagen type I and fibronectin staining for the data shown in FIG. 25F-G. FIG. 25J-M show that pancreatic duct ligation (PDL) at the tail region of the pancreas induced chronic pancreatitis and fibrosis in WT and TRPV4-KO mice. Eight days after PDL, chronic pancreatitis and fibrosis parameters of the tail region included (FIG. 25J) H&E staining, (FIG. 25K) H&E score, (FIG. 25L) Masson's trichrome staining, and (FIG. 25M) area of WT and TRPV4-KO mice (n=5). Statistical comparisons were made using 2-tailed Student's t test. ***P≤0.001. Scale bar: 100 μm.

FIG. 26 shows that the Piezo1 agonist, Yoda1, upregulates TRPV4 expression in PSCs. Shown are mRNA levels of TRPV4 in human PSCs 24 hr after treatment with Yoda1 (5 μm and 25 μM). *P<0.05, **P<0.01, n=5.

FIG. 27 shows a model of mechanosensing in pancreatic ductal adenocarcinoma (PDAC) growth and pro-metastatic niche formation. Development of PDAC exerts mechanical force that activates PSCs (beige) via the mechanically activated ion channel, Piezo1. Piezo1, in turn, activates TRPV4 converting PSCs into cancer-associated fibroblasts (CAFs). High extracellular matrix production by CAFs increases stromal growth. CAFs produce IL-6, IL-8, and other factors which induce a pro-metastatic niche via hepatocyte activation and recruiting myeloid cells rendering the liver receptive to PDAC metastases.

FIG. 28 shows that Piezo1 activates stellate cells and causes pancreatic fibrosis. mRNA levels of IL-6 in human stellate cells were measured 24 hr after treatment with Yoda1 (25 μM).

FIG. 29A-B show that the Piezo1 agonist, Yoda1, reduces α-smooth muscle actin (ACTA2) and increases CD10 expression in human stellate cell FIG. 29A shows quiescent human PSCs containing perinuclear fat droplets (stained green with bodipy dye) cultured on a Matrigel-coated plate. The PSCs became activated (loss of fat droplets) with Yoda1 treatment for 24 hr. FIG. 29B shows mRNA levels of ACTA2 and CD10 in human PSCs 24 hr after Yoda1 treatment.

FIG. 30 shows that the Piezo1 agonist, Yoda1, increases the nuclear localization of the transcriptional coactivator Yes-associated protein (YAP). Mouse stellate cells isolated from wild-type and Piezo1^{GFAP}-KO mice were treated with Yoda1 (25 μM) for 4 hr and the cells were incubated with YAP antibody for 1 hr. The nuclear localization of YAP was visualized with a fluorescent-labeled secondary antibody against YAP. Increased YAP immunofluorescence was observed in PSCs from wild-type, but not Piezo1^{GFAP}-KO mice. *** P<0.001, n=50 cells/group.

FIG. 31A-D show that the genetic deletion of Piezo1 in pancreatic stellate cells reduces pancreatic cancer metastases. KPCY cells were injected into the pancreas of wild-type or Piezo1^{GFAP}-KO mice, which were analyzed 28 days later. FIG. 31A shows that immunofluorescence staining of liver revealed metastatic KPCY cells (red) in wild-type, but not in Piezo1^{GFAP}-KO mice. Nunc blue staining in blue. FIG. 31B shows that serum IL-6 levels were elevated in wild-type mice. FIG. 31C-D show that liver serum amyloid a1 and a2 (saa1 and saa2, respectively) expression levels were elevated in wild-type mice, consistent with induction of a pro-metastatic niche.

FIG. 32 shows an experimental model to study the effects of Piezo1 or TRPV4 on liver pro-metastatic niche formation and pancreatic cancer metastases. Elevated intrapancreatic pressure is produced by ligating the tail region of the pancreas or injecting KPC cells (100,000 cells in 25 μL phosphate buffered saline pH 7, containing 1% Matrigel) into the tail region of the pancreas of 12-16-week-old wild-type, Piezo1^{GFAP}-KO, and TRPV4 KO mice. On day 10, KPCY cells (KPC cells expressing yellow fluorescent protein) are injected into the spleen (500,000 KPCY cells in 50 μL phosphate buffer saline pH 7.4, containing 1% Matrigel). On day 20, the liver and pancreas tissue are processed for microscopic images and quantification of mRNA levels. Serum samples are used for the quantification of pro-metastatic factors.

FIG. 33 shows an experimental design to study the effects of TRPV4 in liver pro-metastatic niche formation and pancreatic cancer metastases. In this experimental model, KPCY cells (100,000 cells in 25 μL phosphate buffer saline pH 7, containing 1% Matrigel) are injected into the tail region of the pancreas with and without a TRPV4 blocker to investigate if a TRPV4 inhibitor can inhibit PDAC growth and stop cancer cell metastasis. For accurate and continuous dosing, the TRPV4 blocker is placed in an infusion osmotic minipump (ALZET) implanted subcutaneously. Mice are observed for 20 days. On day 20, liver and pancreas tissue are processed for microscopic imaging and quantification of mRNA levels. Serum samples are collected for analysis and quantification of pro-metastatic factors.

FIG. 34A-B show that the TRPV4 blocker (GSK2193874) inhibited PDAC growth (FIG. 34A) and reduced pro-metastatic factor SAA in serum (FIG. 34B). Tumor growth and serum SAA were measured 20 days after transplantation of KPCY cells into the pancreas, as described in FIG. 33. Mice were treated with GSK2193874 (20 mg/mL) dissolved in 6% cavitron saline buffer and 100 μL drug solution was loaded in an osmotic minipump implanted subcutaneously for 20 days.

FIG. 35A-E show that the genetic deletion of TRPV4 reduces pancreatic cancer growth and pro-metastatic factors. KPCY cells were injected into the pancreas of wild-type or TRPV4 KO mice, which were analyzed 20 days later. FIG. 35A shows pancreatic tumor images from wild-type and TRPV4 KO mice. The results show that tumor weight (FIG. 35B), serum IL-6 levels (FIG. 35C), and liver serum amyloid a1 and a2 expression (FIG. 35D-E) were elevated in wild-type mice, but not in TRPV4 KO mice.

FIG. 36A-E show that TRPV4 KO mice exhibit reduced metastatic liver colonization. KPC cells were injected into the tail region of the mouse pancreas to initiate PDAC. Ten days later, these PDAC mice were intrasplenically injected with KPCY cells, and the liver was analyzed after ten days. FIG. 36A shows images of the liver showing metastatic tumor lesions in WT mice (areas are marked with a dotted yellow circle). Metastatic tumor lesions were not seen in TRPV4 KO mice. Scale bar=10 mm. FIG. 36B-C show microscopic images of the liver stained for the proliferation marker Ki-67 (magenta) (FIG. 36B) and colonization of KPCY cancer cells (green) (FIG. 36C). Nuclei stained with Nunc blue. Scale bar for FIG. 36B is 100 μm and for FIG. 36C is 50 μm. FIG. 36D shows a graph of Ki67+ cells in WT and TRPV4 KO mice. FIG. 36E shows a graph of the number of lesions in WT and TRPV4 KO mice.

FIG. 37A-B show prophetic experimental designs to study the effects of Piezo1 and TRPV4 in a genetically engineered KPC (KrasLSL-G12D; p53LoxP; Pdx1-CreER) mouse model of PDAC. FIG. 37A shows that tamoxifen (100 mg/kg/day for five days) are injected intraperitoneally to KPC, KPC-Piezo1^{GFAP}-KO, and KPC-TRPV4 KO mice to induce pancreatic tumor development. 8-10 weeks after tamoxifen (around 80 days), the mice should develop pancreatic intraepithelial neoplasia (PanIN) or precursors lesions. FIG. 37A shows that on days 80, 100, and 120 after tamoxifen, the liver and pancreas tissue are processed for microscopic imaging and quantification of mRNA levels. Serum samples are used to quantify pro-metastatic factors as depicted in the model. FIG. 37B shows that tamoxifen is injected intraperitoneally to KPC mice. One group of mice is administered a TRPV4 blocker via an infusion osmotic minipump (ALZET) implanted subcutaneously on day 100 for 20 days and another group of mice are used as a control without TRPV4 blocker. On days 80, 100, and 120 after tamoxifen, the liver and pancreas tissue are processed for microscopic imaging and quantification of mRNA levels. Serum samples are used to quantify pro-metastatic factors.

FIG. 38A-B show immunohistochemistry and immunostaining of human keloid. FIG. 38A shows immunohistochemistry (hematoxylin and eosin staining) and immunostaining image of type I collagen (red) and nuclei (stained with Nunc blue) (blue) in human keloid. FIG. 38B shows immunostaining of TRPV4 protein (red) and Nunc blue-stained nuclei (blue) in human keloid.

FIG. 39 shows immunostained images of GFAP (green), Nunc blue-stained nuclei (blue), and Piezo1 protein (red) in human keloid. Cells positive for both Piezo1 and GFAP are yellow in color.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, reducing the likelihood of developing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of,” “reducing the likelihood of developing,” or “preventing” a disease, disorder, or the symptoms thereof involves administering a cell, composition, or compound described herein to a subject prior to onset of the disease, disorder, or the symptoms thereof. Further, prophylaxis of,” “reducing the likelihood of developing,” or “preventing” also refers to halting or slowing the progression of a disease or disorder after incipient clinical manifestations. “Suppressing” a disease or disorder involves administering a cell, composition, or compound described herein to a subject after induction of the disease or disorder thereof but before its clinical appearance or symptoms thereof have manifest.

The present disclosure is based, in part, on the discovery that mechanical pressure converts pancreatic stellate cells from a quiescent to a fibrogenic/inflammatory phenotype, and that the effects of pressure occur through the mechanically sensitive ion channel, Piezo1, and subsequent stimulation of the ion channel, transient receptor potential vanilloid 4 (TRPV4). Based on these findings, it was further discovered that blockade of either Piezo1 or TRPV4, or combinations thereof, prevents stellate cell activation and the fibrogenic and inflammatory responses, thereby providing for the prevention and treatment of pancreatic diseases and disorders, such as pancreatitis and pancreatic cancer.

One embodiment described herein is one or more inhibitory molecules capable of preventing pancreatic stellate cell activation or activation of a fibrinogenic/inflammatory phenotype in a subject. In some embodiments, the inhibitory molecule comprises an antagonist. In one embodiment, inhibitory molecule comprises a Piezo1 inhibitor. In certain embodiments, the Piezo1 inhibitor comprises GsMTx-4. In another embodiment, the inhibitory molecule comprises a TRPV4 inhibitor. In certain embodiments, the TRPV4 inhibitory molecule is selected from one or more of Ruthenium Red, RN-1734, HC-067047, RN-9893, or combinations thereof.

Another embodiment described herein is a pharmaceutical composition comprising, consisting of, or consisting essentially of one or more inhibitory molecules as provided herein and a pharmaceutically acceptable carrier and/or excipient.

Another embodiment described herein is a method of preventing and/or treating a subject suffering from a pancreatic disease or disorder comprising, consisting of, or consisting essentially of administering to the subject a therapeutically effective amount of one or more inhibitory molecules as provided herein capable of preventing stellate cell activation and/or activation of a fibrinogenic/inflammatory phenotype such that the pancreatic disease and/or disorder is prevented and/or treated in the subject.

In one embodiment described herein, the pancreatic disease and/or disorder comprises pancreatic cancer. In another embodiment, the pancreatic disease and/or disorder comprises pancreatic cancer and/or metastatic pancreatic cancer. In other embodiments, the pancreatic disease and/or disorder comprises pancreatitis.

In another described herein, the method further provides administering to the subject one or more additional therapeutics. In some embodiments, the one or more additional therapeutics is selected from the group consisting of chemotherapeutic agents, anticancer agents, anti-inflammatory agents, antibiotics, steroids, and combinations thereof.

In another embodiment described herein, the one or more additional therapeutics is administered before the one or more inhibitory molecules as provided herein. In another embodiment, the one or additional therapeutics is administered concurrently with the one or more inhibitory molecules as provided herein. In other embodiments, the one or more additional therapeutics is administered after the one or more inhibitory molecules as provided herein.

In one embodiment the method comprises administering a Piezo1 antagonist. In one aspect, the Piezo1 antagonist comprises GsMTx-4.

### GsMTx-4

Grammostola spatulata spider venom peptide toxin

(SEQ ID NO: 1)
GCLEFWWKCNPNDDKCCRPKLKCSKLFKLCNFSF

Disulfide bonds between Cys2-Cys17, Cys9-Cys23, and Cys16-Cys30; C-terminal amidation of Phe34.

In another embodiment the method comprises administering a TRPV4 antagonist. In one aspect, the TRPV4 antagonist comprises Ruthenium Red, RN-1734, HC-067047, RN-9893, GSK2798745, GSK2193874, or combinations thereof.

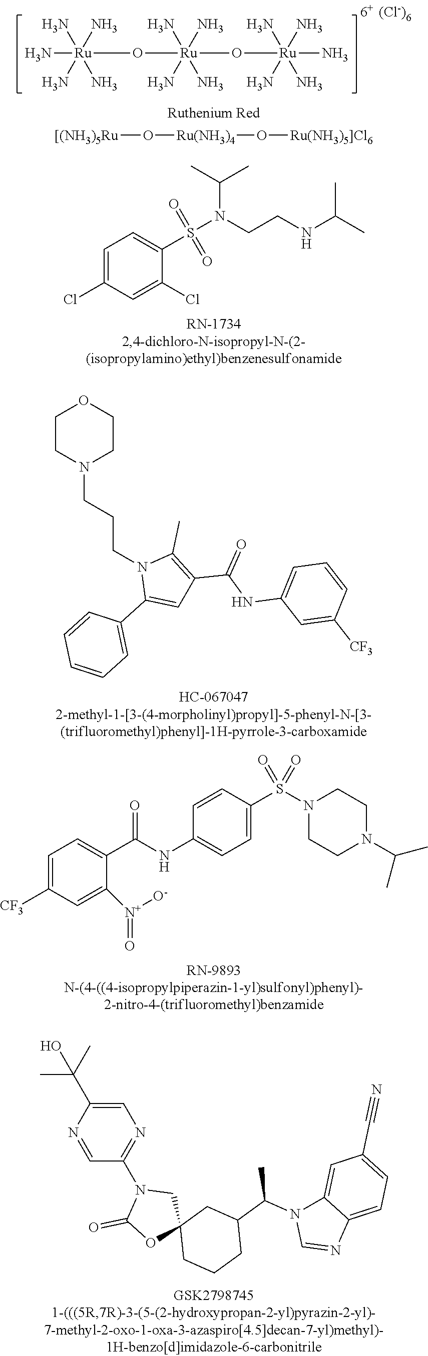

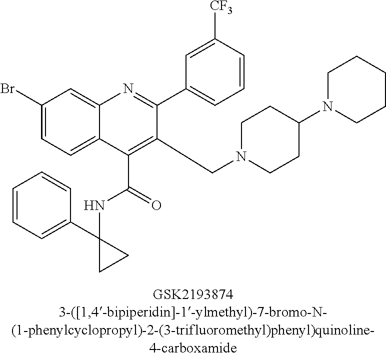 [SMILES: O=C(NC1(c2ccccc2)CC1)c1c(CN2CCC(N3CCCCC3)CC2)c(-c2cccc(C(F)(F)F)c2)nc2cc(Br)ccc12]

In another embodiment the method comprises administering a PLA2 antagonist. In one aspect, the PLA2 antagonist comprises YM26734, AACOCF₃, or a combination thereof.

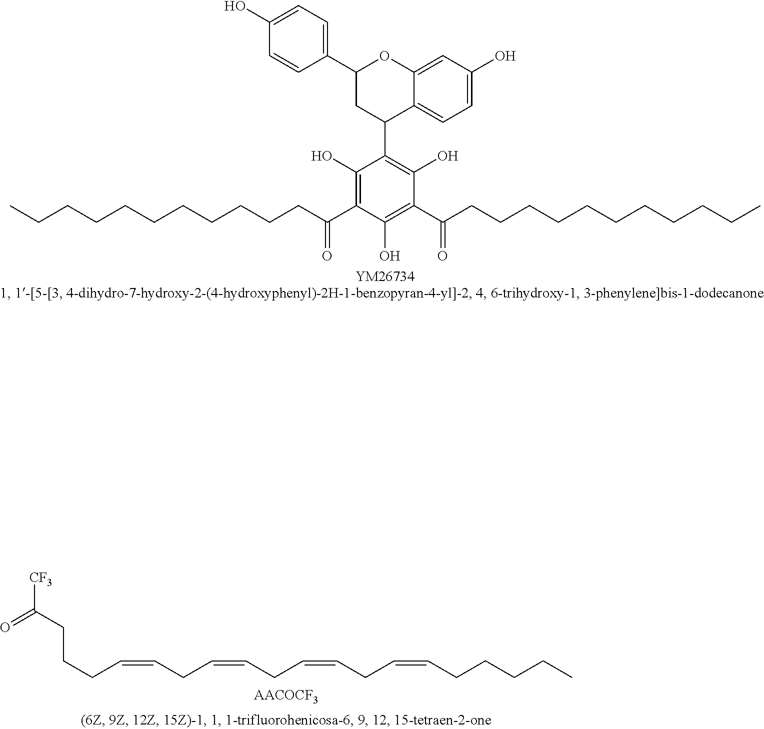

### Pharmaceutically Acceptable Salts

Disclosed compounds (e.g., inhibitory molecules comprising Piezo1 antagonists, PLA2 antagonists, TRPV4 antagonists, or combinations thereof) may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting the compound with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of the carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### Pharmaceutical Compositions

Inhibitory molecules comprising Piezo1 antagonists, PLA2 antagonists, TRPV4 antagonists, combinations thereof, or pharmaceutically acceptable salt thereof may present in a pharmaceutical composition comprising one or more of the antagonists or a pharmaceutically acceptable salts thereof, and a pharmaceutically acceptable carrier, diluent, or excipient. In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

The pharmaceutical composition in various aspects may comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, 18^{th} Edition, E. W. Martin (Mack Publishing Co., Easton, Pa., 1980), which is incorporated by reference in its entirety.

In some aspects, the pharmaceutical composition comprises formulation materials that are nontoxic to recipients at the dosages and concentrations employed. In specific embodiments, pharmaceutical compositions comprising Piezo1 antagonists, PLA2 antagonists, TRPV4 antagonists, or combinations thereof or pharmaceutically acceptable salts thereof, and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In some instances, the pharmaceutical composition may comprise formulation materials for modifying, maintaining, or preserving, for example, the pH, osmolarity, viscosity, clarity, color, isotonicity, odor, sterility, stability, rate of dissolution or release, adsorption, or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; syrup and other carbohydrates (such as glucose, mannose or dextrins); sugar-free syrup; proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapol); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, Remington's Pharmaceutical Sciences, 18^{th} Edition, (A. R. Genrmo, ed.), 1990, Mack Publishing Company.

The pharmaceutical compositions in various instances are formulated to achieve a physiologically compatible pH. In exemplary embodiments, the pH of the pharmaceutical composition is for example between about 4 or about 5 and about 8.0 or about 4.5 and about 7.5 or about 5.0 to about 7.5. In exemplary embodiments, the pH of the pharmaceutical composition is between 5.5 and 7.5.

The pharmaceutical composition may be administered to a subject via parenteral, nasal, oral, pulmonary, topical, vaginal, rectal, or cerebrospinal fluid (CSF) administration. For example, parenteral administration includes intrathecal, intracerebroventricular, intraparenchymal, intravenous, and a combination thereof. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous.

Piezo1 antagonists, PLA2 antagonists, TRPV4 antagonists, combinations thereof, or pharmaceutically acceptable salts thereof may be administered with a physiologically acceptable diluent in a pharmaceutical carrier, such as a sterile liquid or mixture of liquids, including, without limitation, water, saline, aqueous dextrose, and related sugar solutions, syrup including sugar-free syrup, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include, without limitation, petroleum, animal, vegetable, or synthetic oils. Specific examples of oils include peanut, soybean, sesame, cottonseed, corn, olive, petrolatum, and mineral. Suitable fatty acids for use in parenteral formulations include, without limitation, oleic acid, stearic acid, and isostearic acid. Ethyl oleate and isopropyl myristate are examples of suitable fatty acid esters.

Suitable soaps for use in parenteral formulations include, without limitation, fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments may contain Piezo1 antagonists, PLA2 antagonists, TRPV4 antagonists, combinations thereof, or pharmaceutically acceptable salts thereof in solution. Preservatives and buffers can be used. In order to minimize or eliminate irritation at the site of injection, such compositions can contain one or more nonionic surfactants having a hydrophile-lipophile balance (HLB) of from about 12 to about 17. Suitable surfactants include, without limitation, polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions may be prepared from sterile powders, granules, and tablets of the kind previously described.

The pharmaceutical compositions or a pharmaceutically acceptable salt thereof may be present in an injectable formulation. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., 622-630 (1986)).

Formulations suitable for oral administration in some aspects comprise (a) liquid solutions, such as an effective amount of Piezo1 antagonists, PLA2 antagonists, TRPV4 antagonists, combinations thereof, or pharmaceutically acceptable salts thereof dissolved in diluents, such as water, saline, syrups or juice; (b) capsules, sachets, tablets, lozenges, and troches, each containing a predetermined amount of Piezo1 antagonists, PLA2 antagonists, TRPV4 antagonists, combinations thereof, or pharmaceutically acceptable salts thereof, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients.

### Dosings

Piezo1 antagonists, PLA2 antagonists, TRPV4 antagonists, combinations thereof, or pharmaceutically acceptable salts thereof may be administered as a single daily dose. Piezo1 antagonists, PLA2 antagonists, TRPV4 antagonists, combinations thereof, or pharmaceutically acceptable salts thereof, may be administered at a dose of 0.1-200 mg/kg/day. In various instances, Piezo1 antagonists, PLA2 antagonists, TRPV4 antagonists, combinations thereof, or pharmaceutically acceptable salts thereof may be administered at a dose of 1 mg/kg/day to 200 mg/kg/day; 10 mg/kg/day to 100 mg/kg/day; 20 mg/kg/day to 100 mg/kg/day; or 50 mg/kg/day to 10 mg/kg/day. In various instances, Piezo1 antagonists, PLA2 antagonists, TRPV4 antagonists, combinations thereof, or pharmaceutically acceptable salts may be administered at a dose of no greater than 0.1 mg/kg/day; no greater than 0.5 mg/kg/day; no greater than 1 mg/kg/day; no greater than 10 mg/kg/day; no greater than 25 mg/kg/day; no greater than 50 mg/kg/day; no greater than 75 mg/kg/day; no greater than 100 mg/kg/day; no greater than 125 mg/kg/day; no greater than 150 mg/kg/day; no greater than 175 mg/kg/day; or no greater than 200 mg/kg/day. In various instances, Piezo1 antagonists, PLA2 antagonists, TRPV4 antagonists, combinations thereof, or pharmaceutically acceptable salts may be administered at a dose of no less than 0.1 mg/kg/day; no less than 0.5 mg/kg/day; no less than 1 mg/kg/day; no less than 10 mg/kg/day; no less than 25 mg/kg/day; no less than 50 mg/kg/day; no less than 100 mg/kg/day; no less than 150 mg/kg/day; no less than 175 mg/kg/day; or no less than 200 mg/kg/day.

### Diseases and Disorders

The disease or disorder may be associated with fibrogenic and inflammatory proteins and abnormal collagen production. In various instances, the disease or disorder may be associated with one or more of Piezo1, PLA2, TRPV4, phospholipase A2, glial fibrillary acidic protein (GFAP), or combinations thereof. In some instances, the disease or disorder involved stellate cells or myofibroblasts. In some aspects, the disease or disorder may be pancreatic diseases or disorders or keloids. In one aspect, the disease or disorder may be treated or prevented by modulating Piezo1 and TRPV4 among other fibrogenic and inflammatory proteins.

One embodiment described herein is a method for treating a subject suffering from or reducing the likelihood of developing a pancreatic disease or disorder, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof. In one aspect, the Piezo1 antagonist comprises GsMTx-4. In another aspect, the TRPV4 antagonist comprises Ruthenium Red, RN-1734, HC-067047, RN-9893, GSK2798745, GSK2193874, or combinations thereof. In another aspect, the PLA2 antagonist comprises YM26734, AACOCF₃, or a combination thereof. In another aspect, the pancreatic disease or disorder comprises pancreatitis, pancreatic fibrosis, pancreatic cancer, metastatic pancreatic cancer, or combinations thereof. In another aspect, the pancreatic cancer or the metastatic pancreatic cancer comprises pancreatic ductal adenocarcinoma (PDAC). In another aspect, the method of further comprises administering one or more additional therapeutic agents to the subject. In another aspect, the one or more additional therapeutic agents is selected from chemotherapeutic agents, anticancer agents, anti-inflammatory agents, antibiotics, steroids, or combinations thereof. In another aspect, the one or more additional therapeutic agents is administered to the subject before administration of the pharmaceutical composition comprising the inhibitory molecule. In another aspect, the one or more additional therapeutic agents is administered to the subject concurrently with administration of the pharmaceutical composition comprising the inhibitory molecule. In another aspect, the one or more additional therapeutic agents is administered to the subject after administration of the pharmaceutical composition comprising the inhibitory molecule.

Another embodiment described herein is a method for treating or reducing the likelihood of pancreatic stellate cell activation and/or activation of a fibrinogenic or inflammatory phenotype in a subject, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

Another embodiment described herein is a method for treating or reducing the likelihood of a subject developing keloids, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof. In one aspect, the Piezo1 antagonist comprises GsMTx-4. In another aspect, the TRPV4 antagonist comprises Ruthenium Red, RN-1734, HC-067047, RN-9893, GSK2798745, GSK2193874, or combinations thereof. In another aspect, the PLA2 antagonist comprises YM26734, AACOCF₃, or a combination thereof. In another aspect, the method of further comprises administering one or more additional therapeutic agents to the subject. In another aspect, the one or more additional therapeutic agents is selected from anti-inflammatory agents, antibiotics, steroids, or combinations thereof. In another aspect, the one or more additional therapeutic agents is administered to the subject before administration of the pharmaceutical composition comprising the inhibitory molecule. In another aspect, the one or more additional therapeutic agents is administered to the subject concurrently with administration of the pharmaceutical composition comprising the inhibitory molecule. In another aspect, the one or more additional therapeutic agents is administered to the subject after administration of the pharmaceutical composition comprising the inhibitory molecule.

Another embodiment described herein is a method for treating or reducing the likelihood of fibroblast activation and/or activation of a fibrinogenic or inflammatory phenotype in a subject, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

Another embodiment described herein is a pharmaceutical composition for treating a subject suffering from, or reducing the likelihood of developing, a pancreatic disease or disorder or keloid, the pharmaceutical composition comprises one or more inhibitory molecules comprising a Piezo1 antagonist, a PLA2 antagonist, a TRPV4 antagonist, or combinations thereof. In one aspect, the pharmaceutical composition further comprises one or more pharmaceutically acceptable carriers or excipients. In another aspect, the Piezo1 antagonist comprises GsMTx-4. In another aspect, the TRPV4 antagonist comprises Ruthenium Red, RN-1734, HC-067047, RN-9893, GSK2798745, GSK2193874, or combinations thereof. In another aspect, the PLA2 antagonist comprises YM26734, AACOCF₃, or a combination thereof. In another aspect, the pharmaceutical composition further comprises one or more additional therapeutic agents selected from chemotherapeutic agents, anticancer agents, anti-inflammatory agents, antibiotics, steroids, or combinations thereof.

Another embodiment described herein is the use of the pharmaceutical compositions described herein as medicaments for treating a subject suffering from, or reducing the likelihood of developing, a pancreatic disease or disorder or keloid.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A method for treating a subject suffering from or reducing the likelihood of developing a pancreatic disease or disorder, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

Clause 2. The method of clause 1, wherein the Piezo1 antagonist comprises GsMTx-4.

Clause 3. The method of clause 1 or 2, wherein the TRPV4 antagonist comprises Ruthenium Red, RN-1734, HC-067047, RN-9893, GSK2798745, GSK2193874, or combinations thereof.

Clause 4. The method of any one of clauses 1-3, wherein the PLA2 antagonist comprises YM26734, AACOCF₃, or a combination thereof.

Clause 5. The method of any one of clauses 1-4, wherein the pancreatic disease or disorder comprises pancreatitis, pancreatic fibrosis, pancreatic cancer, metastatic pancreatic cancer, or combinations thereof.

Clause 6. The method of any one of clauses 1-5, wherein the pancreatic cancer or the metastatic pancreatic cancer comprises pancreatic ductal adenocarcinoma (PDAC).

Clause 7. The method of any one of clauses 1-6, further comprising administering one or more additional therapeutic agents to the subject.

Clause 8. The method of any one of clauses 1-7, wherein the one or more additional therapeutic agents is selected from chemotherapeutic agents, anticancer agents, anti-inflammatory agents, antibiotics, steroids, or combinations thereof.

Clause 9. The method of any one of clauses 1-8, wherein the one or more additional therapeutic agents is administered to the subject before administration of the pharmaceutical composition comprising the inhibitory molecule.

Clause 10. The method of any one of clauses 1-9, wherein the one or more additional therapeutic agents is administered to the subject concurrently with administration of the pharmaceutical composition comprising the inhibitory molecule.

Clause 11. The method of any one of clauses 1-10, wherein the one or more additional therapeutic agents is administered to the subject after administration of the pharmaceutical composition comprising the inhibitory molecule.

Clause 12. A method for treating or reducing the likelihood of pancreatic stellate cell activation and/or activation of a fibrinogenic or inflammatory phenotype in a subject, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

Clause 13. A method for treating or reducing the likelihood of a subject developing keloids, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

Clause 14. The method of clause 13, wherein the Piezo1 antagonist comprises GsMTx-4.

Clause 15. The method of clause 13 or 14, wherein the TRPV4 antagonist comprises Ruthenium Red, RN-1734, HC-067047, RN-9893, GSK2798745, GSK2193874, or combinations thereof.

Clause 16. The method of any one of clauses 13-15, wherein the PLA2 antagonist comprises YM26734, AACOCF₃, or a combination thereof.

Clause 17. The method of any one of clauses 13-16, further comprising administering one or more additional therapeutic agents to the subject.

Clause 18. The method of any one of clauses 13-17, wherein the one or more additional therapeutic agents is selected from anti-inflammatory agents, antibiotics, steroids, or combinations thereof.

Clause 19. The method of any one of clauses 13-18, wherein the one or more additional therapeutic agents is administered to the subject before administration of the pharmaceutical composition comprising the inhibitory molecule.

Clause 20. The method of any one of clauses 13-19, wherein the one or more additional therapeutic agents is administered to the subject concurrently with administration of the pharmaceutical composition comprising the inhibitory molecule.

Clause 21. The method of any one of clauses 13-20, wherein the one or more additional therapeutic agents is administered to the subject after administration of the pharmaceutical composition comprising the inhibitory molecule.

Clause 22. A method for treating or reducing the likelihood of fibroblast activation and/or activation of a fibrinogenic or inflammatory phenotype in a subject, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

Clause 23. A pharmaceutical composition for treating a subject suffering from, or reducing the likelihood of developing, a pancreatic disease or disorder or keloid, the pharmaceutical composition comprising one or more inhibitory molecules comprising a Piezo1 antagonist, a PLA2 antagonist, a TRPV4 antagonist, or combinations thereof.

Clause 24. The pharmaceutical composition of clause 23, further comprising one or more pharmaceutically acceptable carriers or excipients.

Clause 25. The pharmaceutical composition of clause 23 or 24, wherein the Piezo1 antagonist comprises GsMTx-4.

Clause 26. The pharmaceutical composition of any one of clauses 23-25, wherein the TRPV4 antagonist comprises Ruthenium Red, RN-1734, HC-067047, RN-9893, GSK2798745, GSK2193874, or combinations thereof.

Clause 27. The pharmaceutical composition of any one of clauses 23-26, wherein the PLA2 antagonist comprises YM26734, AACOCF₃, or a combination thereof.

Clause 28. The pharmaceutical composition of any one of clauses 23-27, further comprising one or more additional therapeutic agents selected from chemotherapeutic agents, anticancer agents, anti-inflammatory agents, antibiotics, steroids, or combinations thereof.

Clause 29. Use of the pharmaceutical composition of any one of clauses 23-28 as a medicament for treating a subject suffering from, or reducing the likelihood of developing, a pancreatic disease or disorder or keloid.

### EXAMPLES

### Example 1

### Materials and Methods

### Animals

Targeted deletion of Piezo1 in pancreatic acinar cells was accomplished as follows. Piezo1^{fl/fl} mice were crossed with ptf1^{atm2 (cre/ESR1)Cvw/J} mice (The Jackson Laboratory) to generate the mouse line ptf1a^{CreERTM}; piezo1^{fl/fl}. Piezo1^{fl/fl} mice were used as WT, and ptf1a^{CreERTM}; piezo1^{fl/fl} mice at the age of 5 to 7 weeks were subjected to 1 mg of tamoxifen (Millipore Sigma, T5648) injected intraperitoneally for 5 consecutive days. After tamoxifen induction, the ptf1a^{CreERTM}; piezo1^{fl/fl} mice expressed a truncated Piezo1 specific to pancreatic acinar cells and are referred to as Piezo1^{aci}-KO mice. Eight days after the last tamoxifen injection, mice were used in experiments, and each time a small piece of pancreas was used for genotyping. Mice (both male and female) aged 7 to 12 weeks were used in the experiments. Piezo1^{fl/fl} mice (8 to 12 weeks of age) on a C57BL/6J background were used as WT mice, and ptf1a^{CreERTM}; piezo1^{fl/fl} mice after tamoxifen injection were used as Piezo1^{aci}-KO mice for experiments with the Piezo1 agonist, Yoda1, and shear stress. C57BL/6J mice (8 to 14 weeks old) were used as WT mice, and mice with the trpv4^{-/-} gene deletion (referred to as TRPV4-KO) that were backcrossed on a C57BL/6J background were used for both Yoda1- and partial duct ligation-mediated pancreatitis experiments. For in vivo experiments with Yoda1, WT mice were also injected with tamoxifen. Mice were housed under standard 12-hour light/12-hour dark periods.

### Pancreatic Acini and Acinar Cell Preparations

Mouse pancreatic acini were isolated using a standard collagenase digestion protocol. Isolated acini were plated on a thin-layered, Matrigel-coated, glass-bottom, culture plate (MatTek, P35G-0-14-C). Freshly isolated acini were used in each experiment.

### Shear Stress Assays

Parallel-plate fluid flow chambers (μ-Slide I 0.4 Luer, and μ-Slide I 0.2 Luer from Ibidi GmbH) were used to measure the shear stress-induced changes in intracellular calcium, mitochondrial depolarization, and trypsin activation. The constant flow rate with shear stress (T) was determined as follows: τ=η×104.7.6 φ for μ-Slide I 0.4 Luer and τ=η×330.4 φ for μ-Slide I 0.2 Luer, where η=viscosity of the medium and φ=flow rate (according to the manufacturer's instructions, Ibidi).

### Mechanical Pushing

A borosilicate glass pipette (Sutter Instrument) was pulled using a pipette puller P-87 (Sutter Instruments) and made blunt with an MF-900 Microforge (Narishige). Acini were pushed once with a 2- to 3-μm tip blunt pipette to 5 μm for 1 second using a micromanipulator (World Precision Instruments).

### Calcium Imaging

Calcium imaging in pancreatic acini was performed as previously described. The chemicals used in calcium imaging experiments included the following: Yoda1 (Tocris; 5586), GsMTx4 (Abcam; ab141871), 5′,6′-EET (Santa Cruz Biotechnology; sc-221066), AA (Millipore Sigma; A3611), HC067047 (Tocris; 4100), GSK1016790A (Millipore Sigma; G0798), RN1734 (Tocris; 3746), AACOCF3 (Tocris; 1462), YM26734 (Tocris; 2522), GF109203X (Tocris; 0741), and CCK8 (Sigma-Aldrich).

### Mitochondrial Depolarization

Live-cell mitochondrial depolarization was analyzed using the mitochondrial labeling dye, TMRE. Isolated acini were plated on a thin-layered, Matrigel-coated, glass-bottom culture plate with DMEM/F12 and 10% FBS media and placed in a CO₂ incubator for 1 hr at 37° C. After 1 hr, the acini were incubated with TMRE (200 nM) in the bath buffer containing 140 mM NaCl, 4.7 mM KCl, 2.0 CaCl₂, 1 mM MgCl2, 10 mM HEPES, and 10 mM glucose (pH adjusted to 7.4 with NaOH) for 30 minutes. The TMRE dye was washed and replaced with fresh bath buffer. The images were captured with a Zeiss Axio observer Z1 microscope with MetaMorph software (Molecular Devices) at intervals of 600 ms. TMRE was excited at 540-600 nm and emission collected at 585-675 nm. Carbonyl cyanide 4-(trifluoromethoxy) phenylhydrazone (FCCP), which uncouples the oxidative phosphorylation process and depolarizes mitochondria, was used as a positive control. A maximum of 2 mitochondrial puncta were taken per acinar cell.

### Trypsinogen Activation

To visualize Piezo1-induced trypsinogen activation, acini were loaded with active trypsin enzyme substrate BZiPAR (10 μM). Na-HEPES buffer with 2 mM Ca²⁺ was used during imaging. A Zeiss Axio observer Z1 with a high-sensitivity EMCCD camera with a ×40/0.75 EC-Plan-NeoFluar DIC objective was used to capture the Z-stack images with stack thickness of 3 μm at an interval of 12 seconds. The BZiPAR fluorescent wavelength Ex/Em was 498/521 nm. BZiPAR was excited at 470-510 nm and emission collected at 495-550 nm. The captured images were analyzed with MetaMorph software (Molecular Devices).

### Cell Death Assays

The viability of pancreatic acini with Yoda1 and CCK treatments was analyzed using the Live/Dead Cell Imaging Kit (Thermo Fisher Scientific, catalog R37601) or the LDH Release Assay Kit (Promega, catalog G1780).

### PLA2 Activity

The fluorogenic PLA2 substrate (Bis-BODIPY FL C₁₁-PC) (Thermo Fisher Scientific; B7701) was used to monitor PLA2 activity in living cells. The pancreatic acini were incubated with Bis-BODIPY FL C₁₁-PC in HBSS buffer with 2 mM Ca²⁺ for 30 minutes. HBSS buffer with 2 mM Ca²⁺ was used during imaging. A Zeiss Axio observer Z1 with a high-sensitivity EMCCD camera with a ×40/0.75 EC-Plan-NeoFluar DIC objective was used to capture the Z-stack images with stack thickness of 4 μm at an interval of 10 seconds with 480 nm excitation and 525 nm emission filter. The captured images were analyzed with MetaMorph software (Molecular Devices).

### Immunostaining

Pancreatic acini were incubated with a rabbit anti-TRPV4 antiserum (Alomone; ACC-034; 1:250) or with a rabbit anti-Piezo1 antiserum (Alomone; APC-087; 1:300) overnight at 2° C.-8° C. The signals from TRPV4 and Piezo1 immunostaining were amplified by the tyramide signal amplification method using a kit (Life Technologies; T20924), following the manufacturer's instructions. The nuclei were stained with Nunc blue (Invitrogen; R376060). All staining images were taken with a Zeiss Axio observer Z1 with a ×20 objective or a ×63 oil-immersion objective.

### In Vivo Pancreatitis Models

Laparotomy surgery was performed both in pancreatic partial duct ligation- and retrograde pancreatic duct infusion-mediated pancreatitis experiments as previously described by Romac et al., Nat. Commun. 9(1):1715 (2018). Yoda1 at a dose of 0.4 mg/kg in 50 μL 1.1% dimethylsulfoxide, 4.8% ethanol, and 94.1% buffered saline was injected. In Yoda1-mediated pancreatitis, the midportion of the pancreas (about 100-125 mg) was carefully excised and used for biochemical assays and histological staining. Histological scoring was evaluated from the entire section. Partial PDL was performed as previously described. Using a stereo microscope, the tail region of the pancreas was visualized, and the main pancreatic duct was ligated carefully with 6-0 Prolene suture without damaging the left portal vein that separates the splenic lobe and gastroduodenal part of the pancreas. Any mice suffering damage to any underlying blood vessels were excluded from the experiment. The pancreas was examined 24 hr after ligation. A 100- to 125-g portion of the tail region of the pancreas was used for biochemical assays and histological staining. Mice were subjected to caerulein-induced pancreatitis by intraperitoneal injection of caerulein (50 μg/kg) (Tocris; 6264) every hour for a total of 6 injections.

### Blood Amylase Assay

Serum amylase concentration was measured by a colorimetric method after reaction with substrate using the Phadebas Amylase Test Tablet (Magle Life Sciences) and the Tecan-infinite M200 Pro plate reader.

### MPO Assay

MPO was measured using previously described methods with modifications. See Romac et al., Nat. Commun. 9(1):1715 (2018). To calculate the mU of MPO/mg of protein, the protein concentrations of the supernatants were measured using the Micro BCA Protein Assay Kit (Thermo Fisher Scientific; 23235).

### H&E Staining

In Yoda1-mediated pancreatitis, the body region of the pancreas was used for H&E staining, and in partial duct ligation-mediated pancreatitis, the tail region of the pancreas was used. The histological score was calculated from the pathological parameters, e.g., tissue edema, neutrophil infiltration, necrosis, and hemorrhage, with a minimum to severe scoring range of 0-3, 0-3, 0-7, and 0-7, respectively. The scores from all parameters were added to obtain a total histological score.

### RT-PCR

RNAs were isolated using the RiboPure Kit (Invitrogen; AM1924), followed by DNase I digestion (Invitrogen; AM1906).

### Statistics

Results are expressed as mean±SEM. Mean differences between 2 groups were analyzed by 2-tailed Student's t test, and mean differences between multiple groups were analyzed by 1-way ANOVA with Tukey's multiple comparison posttest (GraphPad Prism 8). P values of less than 0.05 were considered significant.

### Example 2

### Piezo1 Induces Sustained Cytoplasmic Ca²⁺ Elevation and Cell Death

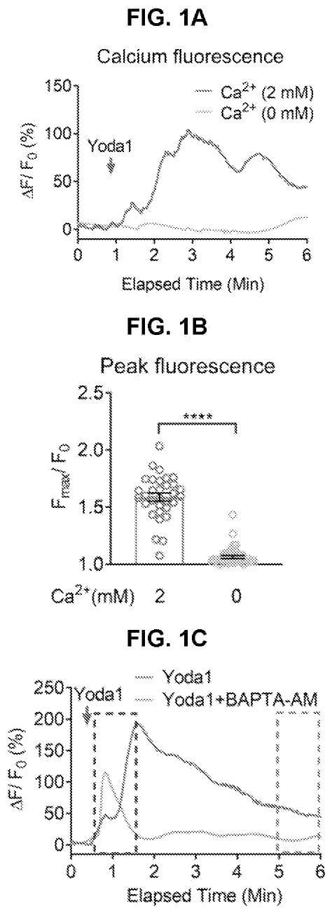

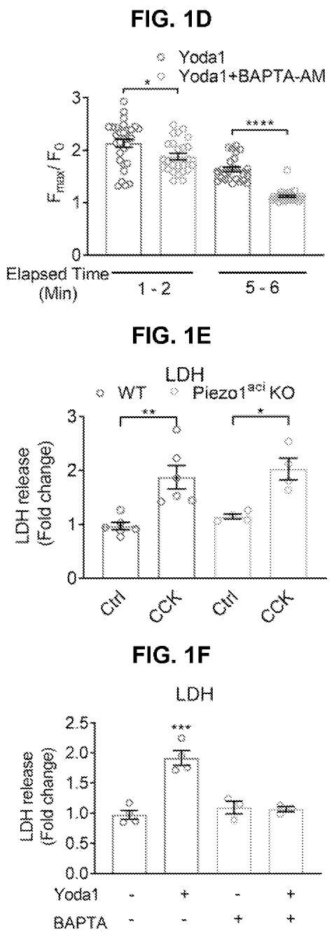

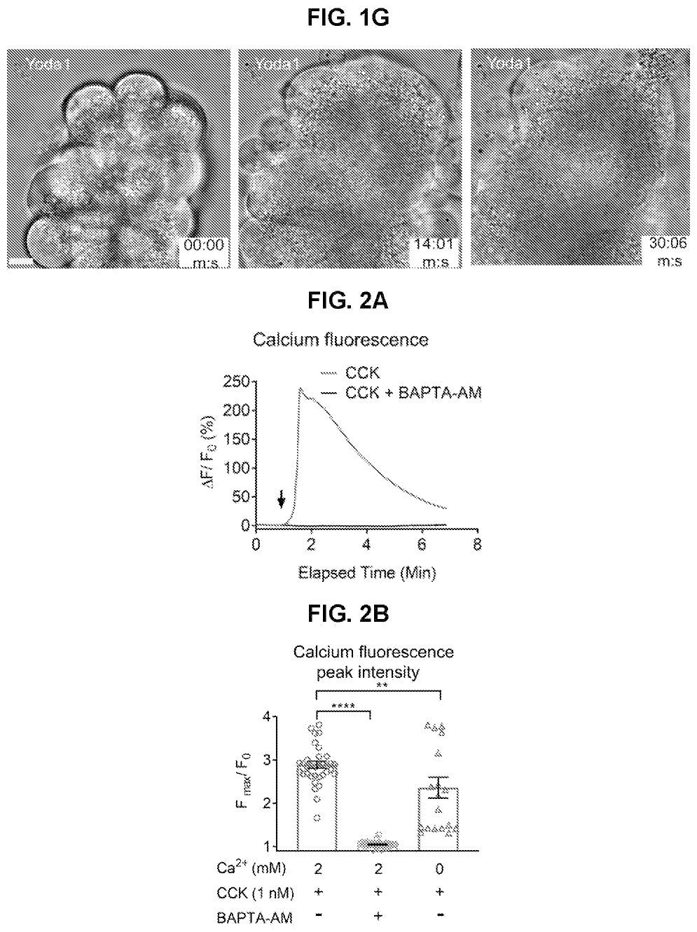

In order to determine whether calcium signaling is responsible for the effects of Piezo1 on the pancreas, the ability of the chemical Piezo1 agonist Yoda1 to regulate intracellular calcium ([Ca²⁺]ᵢ) was first examined in freshly isolated pancreatic acini. Changes in [Ca²⁺]ᵢ were determined in acinar cells loaded with the calcium indicator calcium 6-QF. It was recently reported that the Piezo1 antagonist GsMTx4 inhibits the Yoda1-mediated [Ca²⁺]ᵢ rise in pancreatic acini. Here, the contribution of external and intracellular free calcium on Piezo1-mediated [Ca²⁺]ᵢ is demonstrated. Yoda1 in the presence of bath calcium (2 mM) produced an initial transient Ca²⁺ rise, followed by sustained intracellular calcium elevation (FIG. 1A-B). Removal of external calcium completely abolished the Yoda1-mediated [Ca²⁺]ᵢ rise (FIG. 1A-B). However, preincubating cells with BAPTA-AM (a cell-permeable Ca²⁺ chelator) for 30 minutes abolished the sustained Ca²⁺ rise (FIG. 1C-D). CCK is a secretagogue that stimulates pancreatic enzyme secretion by increasing [Ca²⁺]ᵢ. In contrast to the effects on Yoda1-induced [Ca²⁺]ᵢ, preincubation of cells with BAPTA-AM completely eliminated the effect of CCK on [Ca²⁺]ᵢ (FIG. 2A-B).

To examine the effect of sustained Piezo1 activation on [Ca²⁺]ᵢ and its relation to cellular injury, pancreatic acini were treated with Yoda1, and cellular injury was assessed by measuring lactate dehydrogenase (LDH) release. Cells were preincubated with or without BAPTA-AM. Chelating intracellular free calcium with BAPTA-AM protected pancreatic acini from Yoda1-induced LDH release (FIG. 1F). The specificity of these effects for Piezo1 was confirmed by comparing the cytotoxic effects of CCK (FIG. 1E). At high concentrations, CCK is well known to cause cell damage in vitro and pancreatitis in vivo. As shown in FIG. 1E, CCK produced comparable cell damage in pancreatic acini from both WT mice and mice with selective genetic deletion of Piezo1 in pancreatic acinar cells (Piezo^{aci}-KO mice). The effect of Yoda1 on pancreatic acini was visualized over time by live-cell imaging. Application of Yoda1 (50 μM) caused swelling of WT pancreatic acinar cells and release of zymogen granules from the basolateral surface and gradually ruptured the cell membrane (FIG. 1G). Pancreatic acinar cells from Piezo1^{aci}-KO mice did not respond to Yoda1.

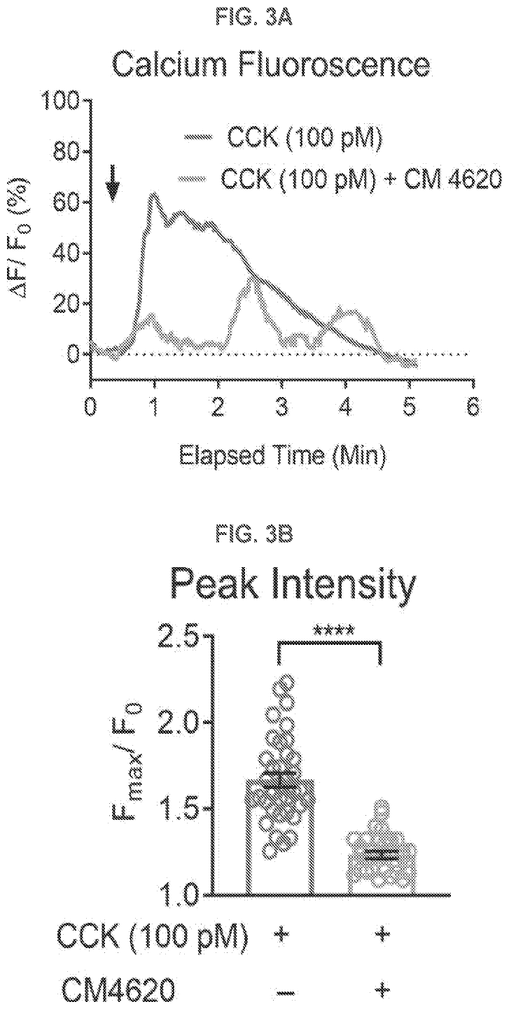

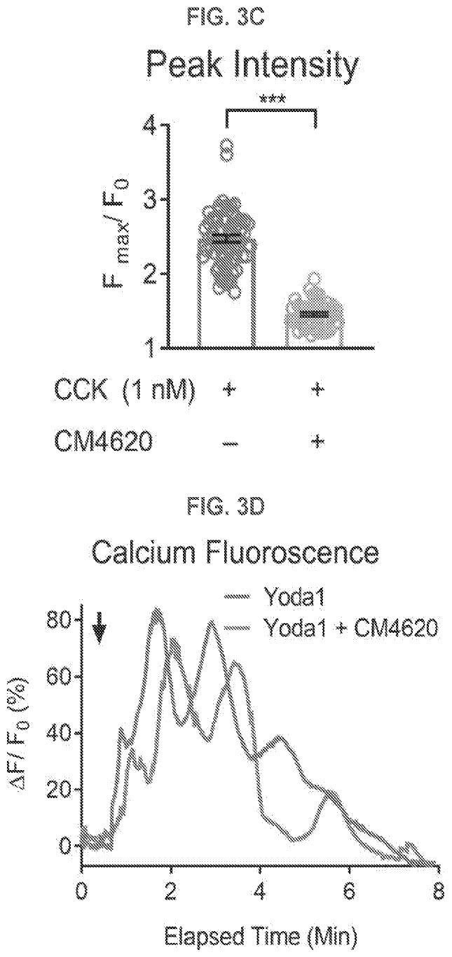

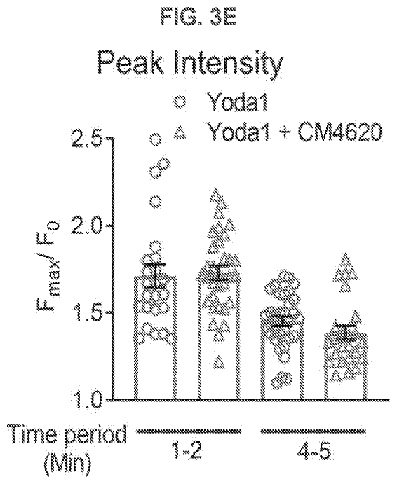

In pancreatic acinar cells, CCK at supraphysiological concentrations produces a sustained elevation of [Ca²⁺]ᵢ, the initial phase of which is due to release of Ca²⁺ from the ER. Following this initial rise, the sustained phase occurs through the activation of CRAC, which allows extracellular Ca²⁺ to flow into cells. In order to determine whether the Piezo1-mediated sustained [Ca²⁺]ᵢ elevation is due to CRAC activation, the effects of the CRAC inhibitor CM4620 was examined, which selectively inhibits Orai, the main component of CRAC. Preincubating acinar cells with CM4620 for 1 hr blocked the sustained elevation in [Ca²⁺]ᵢ produced by CCK (100 and 1000 μM) (FIG. 3A-C). Notably, CM4620 did not completely block the [Ca²⁺]ᵢ rise induced by either concentration of CCK, and a residual calcium wave was always observed following CCK despite CM4620 administration (FIG. 3A-C). This persistent calcium wave was possibly due to Ca²⁺ released from ER stores. In contrast to the effects on CCK-stimulated [Ca²⁺]ᵢ, CM4620 did not alter the rise in [Ca²⁺]ᵢ following Yoda1 stimulation (FIG. 3D-E), indicating that CRAC channels are not the source of sustained [Ca²⁺]ᵢ elevation following Piezo1 activation.

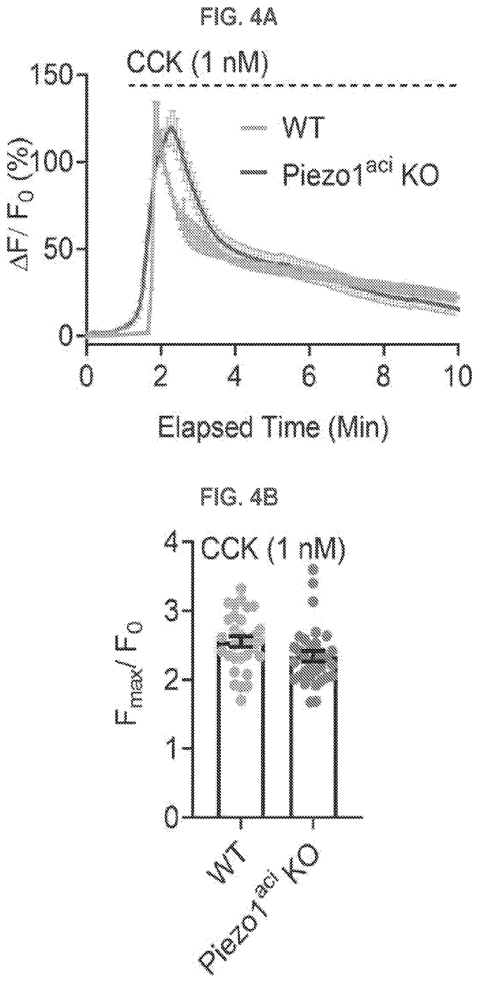

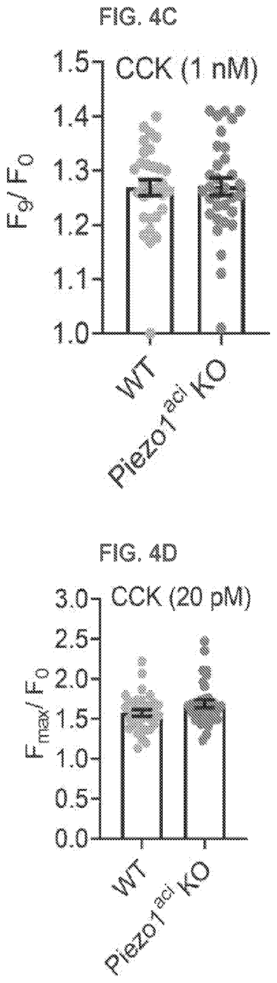

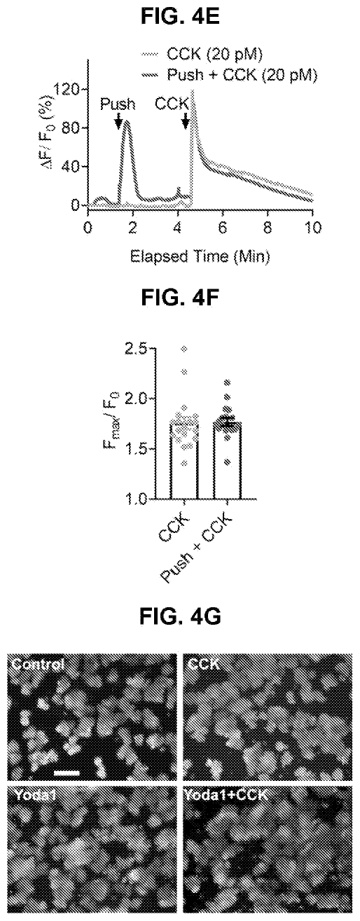

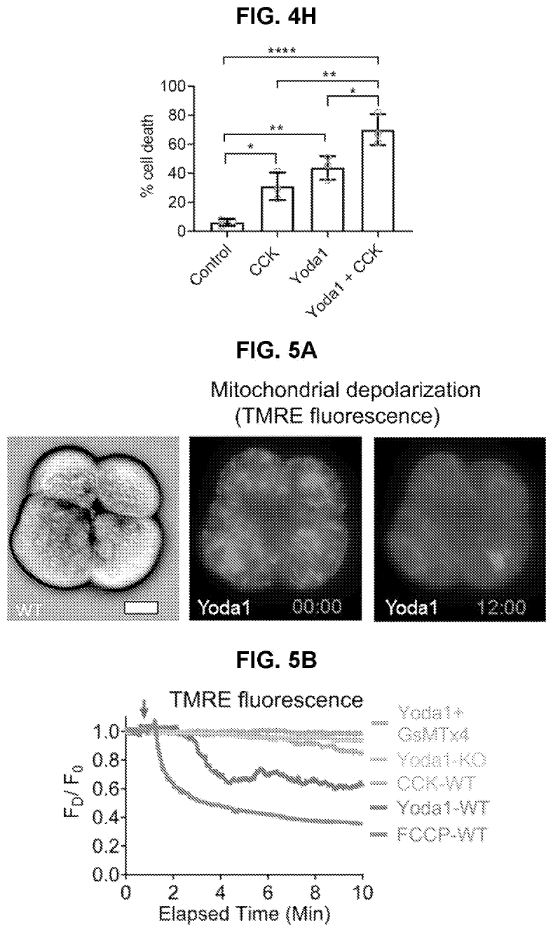

To determine whether Piezo1 gene deletion altered the acinar cell response to secretagogue stimulation, the effects of CCK on [Ca²⁺]ᵢ in pancreatic acini from Piezo1^{aci}-KO mice was examined. Pancreatic acini from WT and Piezo1^{aci}-KO mice responded equally to both physiological (20 μM) and supraphysiological (1 nM) CCK concentrations (FIG. 4A-D). In order to confirm that Piezo1 and CCK stimulate [Ca²⁺]ᵢ through distinct mechanisms, Piezo1 channels were first activated in pancreatic acini through mechanical force by applying a blunt glass pipette to the surface of acinar cells to a depth of 5 μm for 1 second. This blunt pushing produced a transient [Ca²⁺]ᵢ elevation. Cells were then exposed to CCK (20 μM). Slight mechanical pushing did not alter the sensitivity of pancreatic acini to subsequent CCK exposure (FIG. 4E-F). In light of the finding that Piezo1 and CCK affect [Ca²⁺]ᵢ through separate pathways, it was determined whether together Yoda1 and CCK accentuated the deleterious effects on pancreatic acinar cells. Compared with individual application of Yoda1 and CCK, acinar cell death was greater when cells were exposed to both Yoda1 and CCK (FIG. 4G-H).

### Piezo1 Agonist Yoda1 Induces Pathological Events in Pancreatic Acini

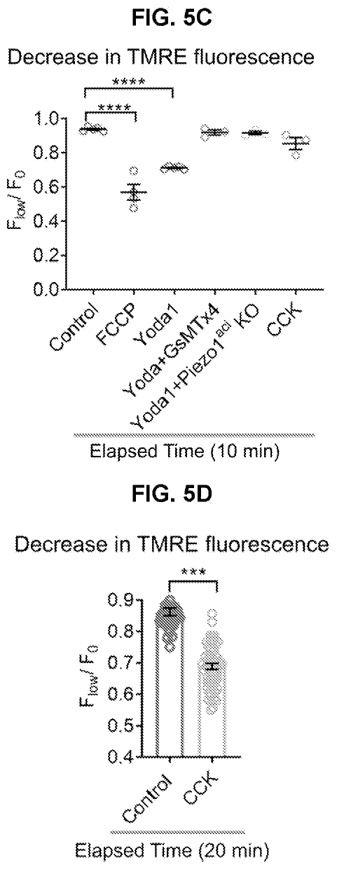

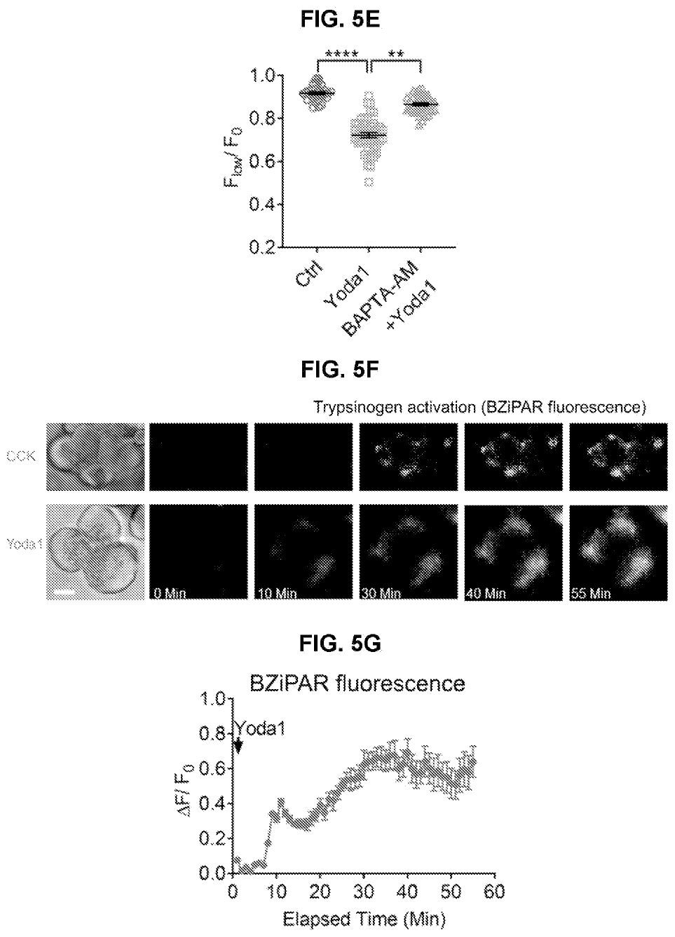

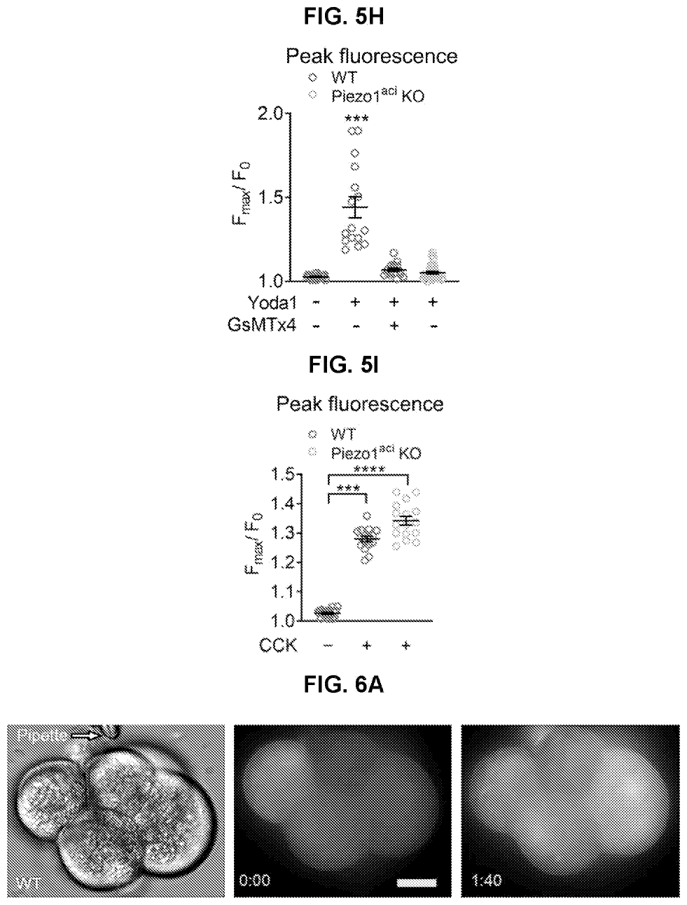

Pancreatic acinar cells possess abundant mitochondria and are highly metabolically active. Proper mitochondrial function is critical for cellular homeostasis, and mitochondrial dysfunction has been implicated in the pathogenesis of pancreatitis. It was postulated that the elevations in cytoplasmic [Ca²⁺] that were observed with Yoda1 stimulation and Piezo1 activation in pancreatic acinar cells may affect mitochondrial depolarization. To test this hypothesis, a cell-permeable and mitochondrial-potential sensitive fluorescent dye was used, tetramethyl rhodamine ester (TMRE). TMRE accumulates as punctate distributions in mitochondria due to high negative membrane potential. The fluorescence intensity of this punctate pattern declines following mitochondrial depolarization. Application of Yoda1 (50 μM) throughout the period of live-cell imaging significantly decreased TMRE fluorescence intensity indicative of depolarization of the mitochondria (FIG. 5A-C). GsMTx4 blocked the Yoda1-induced mitochondrial depolarization in WT cells. Yoda1 did not depolarize mitochondria in Piezo1^{aci}-KO cells (FIG. 5B-C). Pancreatic acinar cells preincubated with BAPTA-AM for 20 minutes were protected from Yoda1-induced mitochondrial depolarization, suggesting a significant role for intracellular-free calcium (FIG. 5E). It was observed that Yoda1-induced mitochondrial depolarization occurred at a higher rate than that induced with CCK (10 nM) (FIG. 5A-D). Yoda1-mediated mitochondrial depolarization occurred within 3 minutes of application; however, CCK-mediated mitochondrial depolarization occurred more slowly and was observed only after 10 minutes of drug exposure.

Premature zymogen activation and autodigestion of acinar cells are critical events in pancreatitis. Moreover, pathophysiological elevations in cytoplasmic [Ca²⁺] have been associated with intracellular trypsinogen activation. It was postulated that prolonged activation of Piezo1 may cause trypsinogen activation through a calcium-mediated pathway. Real-time trypsinogen activation in pancreatic acini was visualized using a trypsin-sensitive, cell-permeable fluorescent probe, rhodamine 110, bis(CBZ-L-isoleucyl-L-prolyl-L-arginine amide) (BZiPAR), which becomes fluorescent once it is cleaved specifically by trypsin. Application of either Yoda1 (50 μM) or CCK (10 nM) activated trypsin in pancreatic acini over time (FIG. 5F-G). Yoda1 induced trypsin activation within 10 minutes of application; however, CCK stimulated trypsin activation more slowly and was observed only after 20 minutes of drug exposure. GSMTx4 blocked the Yoda1-induced trypsin activation in pancreatic acinar cells (FIG. 5H). Pancreatic acinar cells from Piezo1^{aci}-KO mice did not respond to Yoda1 (FIG. 5H). Furthermore, CCK-mediated trypsin activation was similar in acini from WT and Piezo1^{aci}-KO mice (FIG. 51).

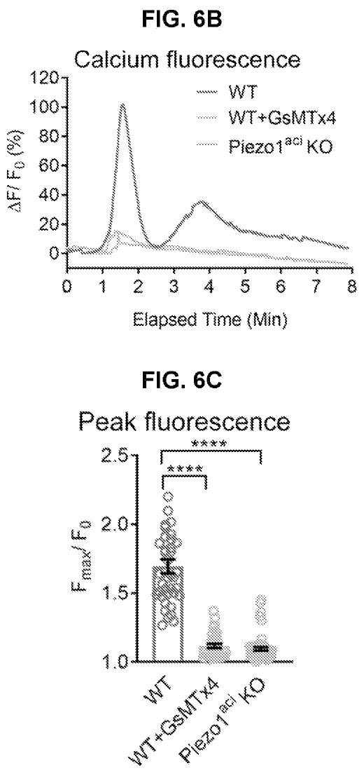

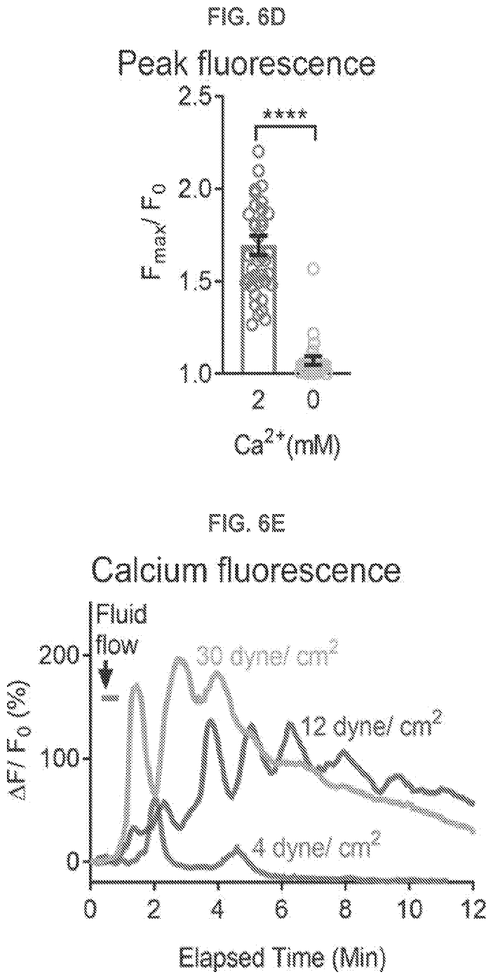

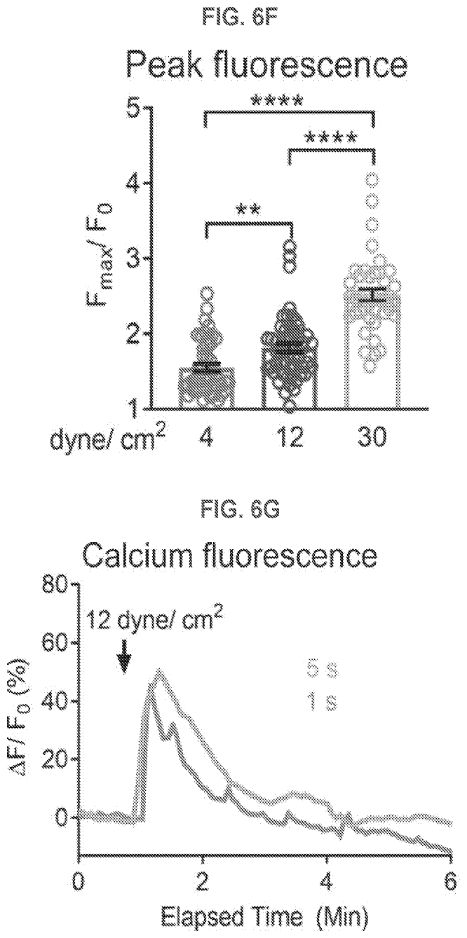

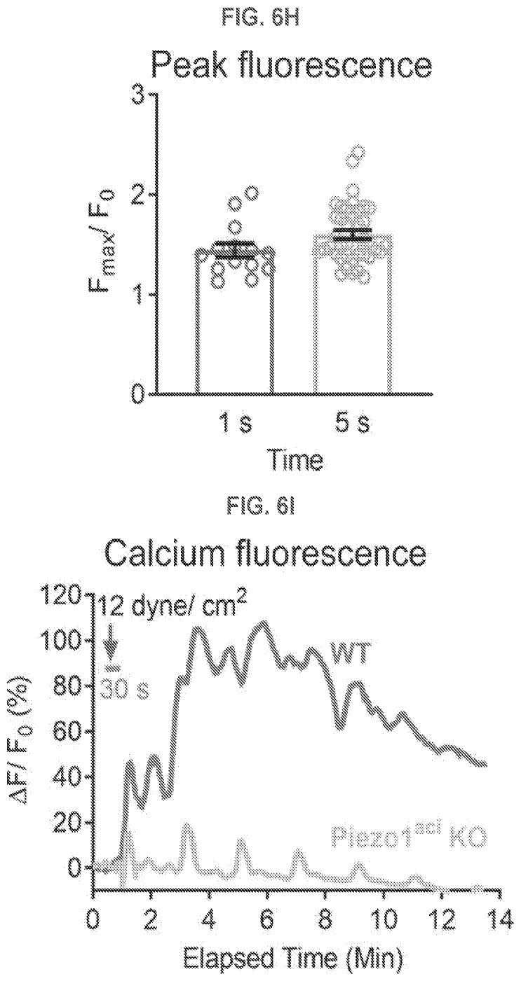

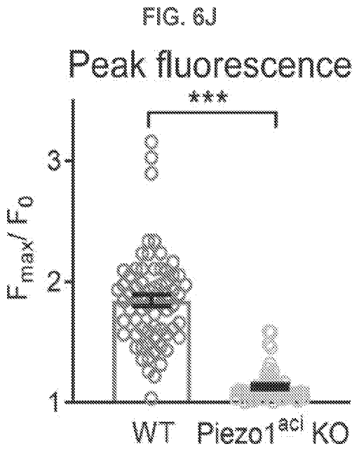

Mechanical Pushing- and Shear Stress-Induced [Ca²⁺]ᵢ Elevation in Pancreatic Acini Mechanical activation of Piezo1 in acinar cells was demonstrated by applying a blunt glass pipette to the surface of acinar cells to a depth of 5 μm for 1 second. This stimulation produced a transient [Ca²⁺]ᵢ rise (FIG. 6A-B) that was completely blocked by prior treatment of the cells with GsMTx4 (FIG. 6C). In addition, mechanical pushing did not increase [Ca²⁺]ᵢ in acinar cells from Piezo1^{aci}-KO mice (FIG. 6C). Removal of external calcium blocked the mechanical pushing-mediated [Ca²⁺]ᵢ rise (FIG. 6D). In order to apply physical force for a longer time, a fluid shear stress approach was used to avoid cell damage that might occur with repeated mechanical pushing and technical difficulties. The fluid shear stress approach is similar to the situation in which high fluid pressure is injected into the pancreatic duct, as it has been demonstrated, which has been used as a model for the clinical condition of excess filling of the pancreatic duct in humans during ERCP.

Mechanical shear stress is a physiological activator of Piezo1 in many tissues, including vascular endothelium. To determine whether Piezo1 channels are responding to fluid shear stress in the pancreas, changes in [Ca²⁺]ᵢ were evaluated in freshly isolated pancreatic acini plated in a shear flow chamber. Following a period as brief as 30 seconds, the peak intensity of [Ca²⁺]ᵢ in pancreatic acini increased as greater shear stress forces were applied (FIG. 6E-F). Shear stresses of 4, 12, and 30 dyne/cm² increased the peak [Ca²⁺]ᵢ by 1.5±0.1-, 1.8±0.1-, and 2.5±0.1-fold, respectively (FIG. 6E-F). The 12 and 30 dyne/cm² stresses caused a sustained elevation in [Ca²⁺]ᵢ and mimicked the [Ca²⁺]ᵢ pattern induced by Yoda1 (25 μM and 50 μM) (FIG. 6E, and FIGS. 1A and 1C). However, the lower shear stress force of 4 dyne/cm² elicited only a transient [Ca²⁺]ᵢ rise (FIG. 6E) without a prolonged [Ca²⁺]ᵢ elevation. Furthermore, fluid shear stress force of 12 dyne/cm² applied for 1 second or 5 seconds was not sufficient to induce a sustained elevation in [Ca²⁺]ᵢ (FIG. 6G-H). The sustained [Ca²⁺]ᵢ elevation at 12 dyne/cm² did not occur in pancreatic acini from Piezo1^{aci}-KO mice, although small transient [Ca²⁺]ᵢ peaks at regular intervals could be seen in some cells (FIG. 6I-J). It was suspected that small transient spikes in [Ca²⁺]ᵢ occurring at regular intervals could be from low expression of other mechanically sensitive channels.

### Fluid Shear Stress Induces Pathological Events in Pancreatic Acini

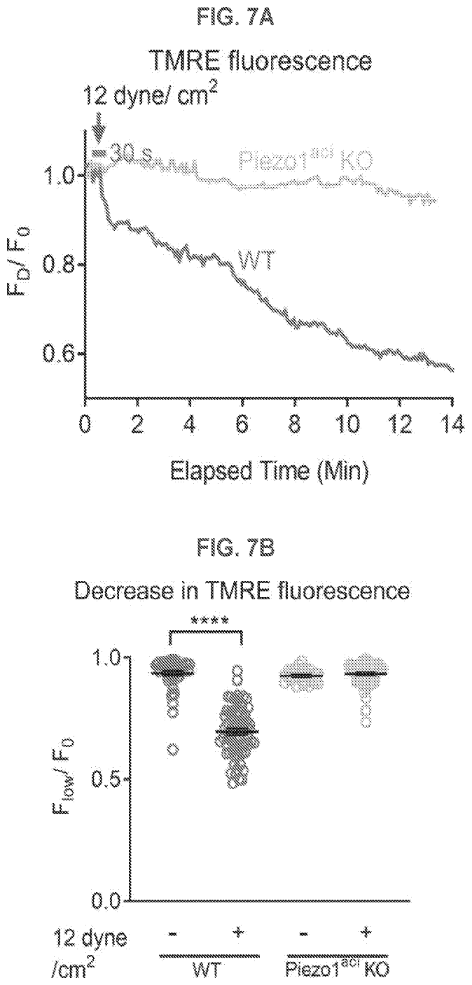

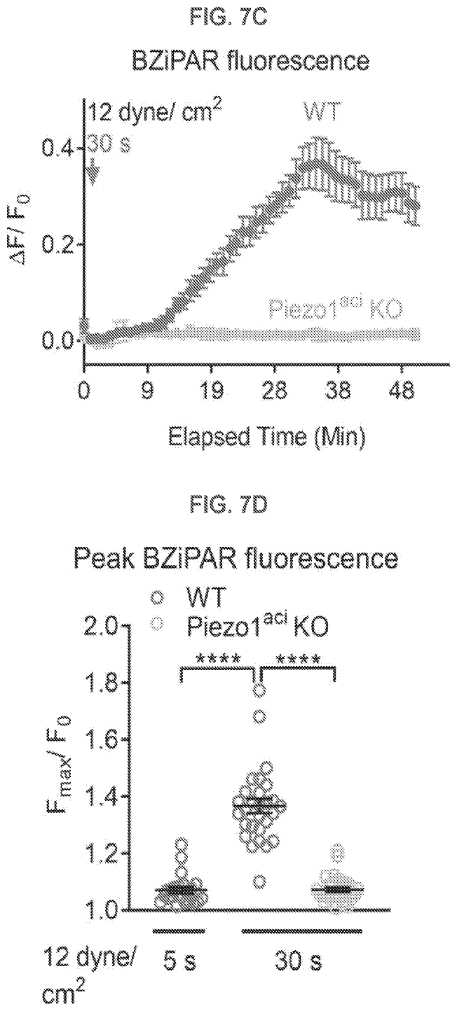

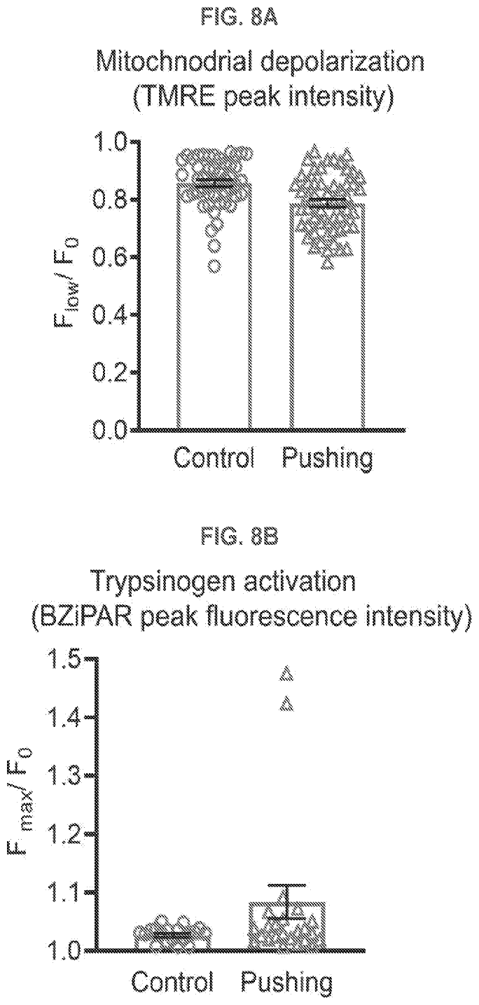

To determine whether fluid shear stress-activated Piezo1 facilitates mitochondrial depolarization, live-cell mitochondrial depolarization was monitored in pancreatic acini loaded with the mitochondrial sequestrant dye TMRE (200 nM) from WT and Piezo1^{aci}-KO mice. Fluid shear stress administered at 12 dyne/cm² for 30 seconds that caused a sustained elevation in [Ca²⁺]ᵢ decreased the TMRE intensity over time in WT pancreatic acini, but not in Piezo1^{aci}-KO cells (FIG. 7A-B). In these experiments, fluid shear stress led to a sustained depolarization consistent with a state of mitochondrial dysfunction. The mitochondrial potential of pancreatic acini with Piezo1 deletion was not affected by these conditions of fluid shear stress (FIG. 7B). These results demonstrate that Piezo1 channels mediate fluid shear stress-induced mitochondrial depolarization. In contrast, mechanical pushing of acinar cells with a glass pipette only slightly depolarized the mitochondria in 20% of acinar cells, while the remainder were completely unaffected. Overall, it appears that pushing of pancreatic acinar cells once for 1 second was not sufficient to substantially depolarize the mitochondria (FIG. 8A).

Pancreatic enzyme activation in pancreatic acinar cells is a key pathological feature in pancreatitis. As disclosed here, the Piezo1 agonist Yoda1 induces trypsinogen activation, which is the initial step in activation of other zymogens in the pancreas. To determine whether fluid shear stress mimics the Yoda1 effect and trypsin activation, pancreatic acinar cells were loaded with the trypsin activity-measuring probe BZiPAR. Pancreatic acini were then subjected to fluid shear stress at 12 dyne/cm² for 30 seconds. Trypsin activity monitored by live-cell imaging was detected after 10 minutes of fluid shear stress and gradually increased for up to 50 minutes (FIG. 7C). An increase in BZiPAR dye (trypsin activity) was not observed in Piezo1^{aci}-KO cells. In contrast, however, a short pulse of 5 seconds, instead of 30 seconds, at a force of 12 dyne/cm² did not trigger trypsin activation in WT acinar cells (FIG. 7D). As was observed with mitochondrial depolarization, brief mechanical pushing of pancreatic acinar cells was not sufficient to activate trypsin (FIG. 8B).

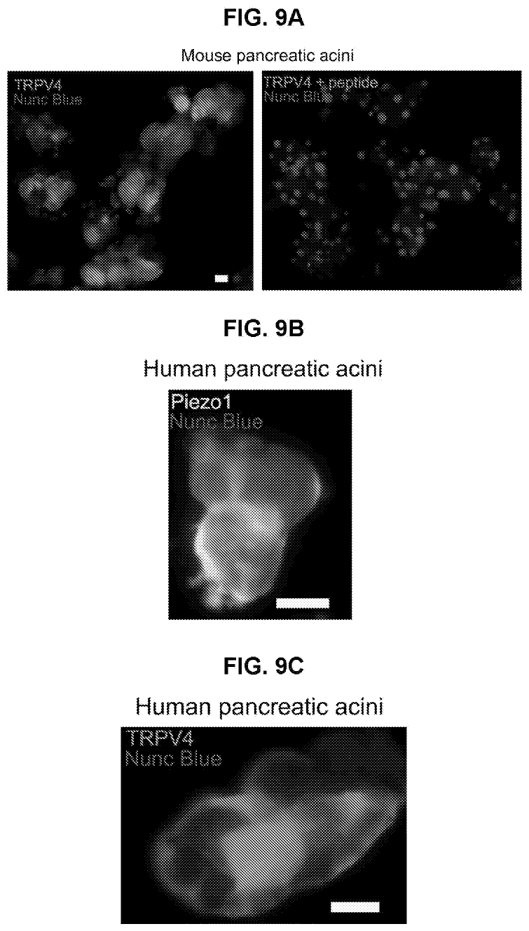

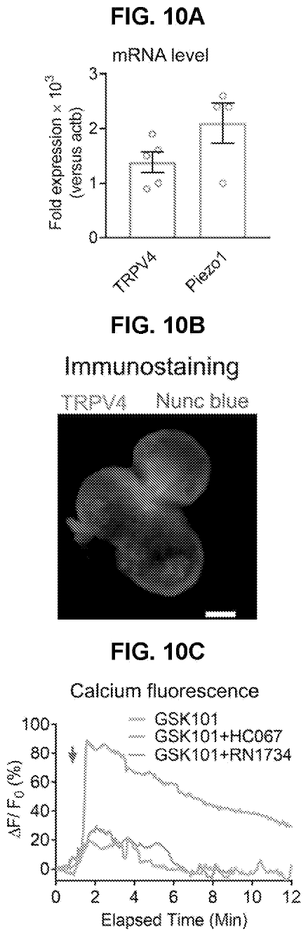

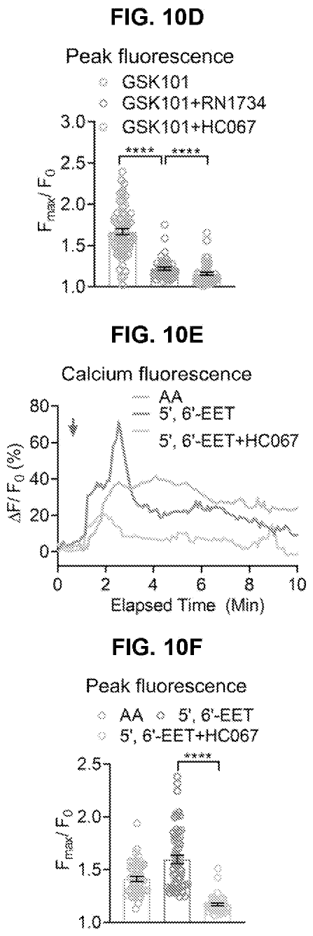

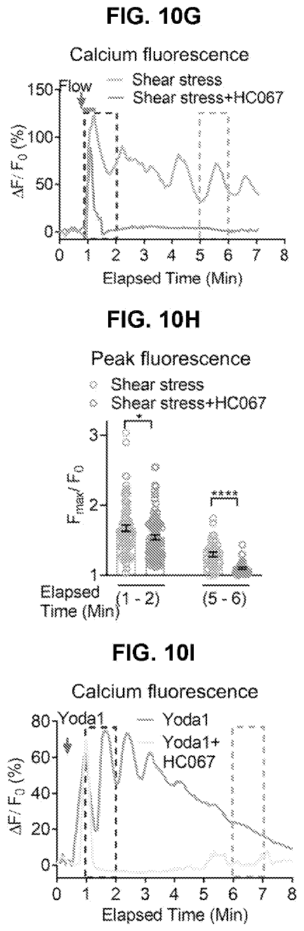

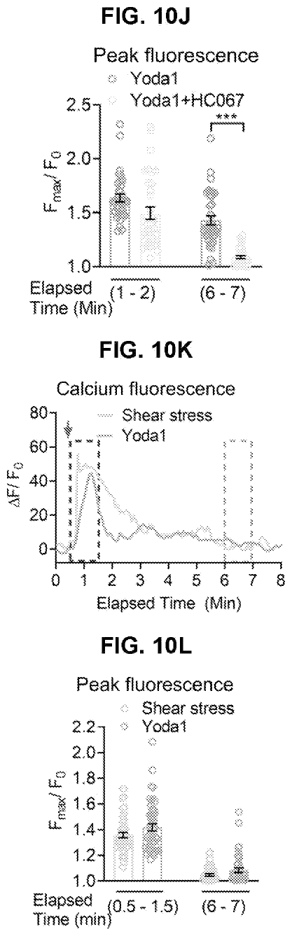

Piezo1 Mediates TRPV4 Channel Activation in Pancreatic Acini Although Piezo1 is a fast-inactivating channel, fluid shear stress and Yoda1 caused a sustained elevation in [Ca²⁺]ᵢ, raising the possibility that other calcium entry channels or Piezo1-mediated downstream signaling pathways may exist. CRAC, which is expressed in pancreatic acinar cells, was one possibility; however, it was observed that the CRAC inhibitor CM4620, which selectively inhibits the Orai channel, blocked the sustained elevation in [Ca²⁺]ᵢ produced by CCK, but not that induced by Yoda1 (FIG. 3). Therefore, it is unlikely that CRAC contributes to Piezo1-stimulated Ca²⁺ entry. Since TRPV4 indirectly senses fluid shear stress, it was determined whether TRPV4 was expressed in pancreatic acinar cells. As shown in FIG. 10A-B, TRPV4 mRNA and protein were detected by quantitative reverse transcriptase PCR (RT-qPCR) and immunostaining, respectively. Moreover, TRPV4 was highly expressed in both mouse and human pancreatic acini (FIG. 10A-B, and FIGS. 9A and 9C). Like mouse pancreatic acini, Piezo1 was also expressed in human pancreatic acini (FIG. 9B). In order to evaluate the function of TRPV4 in pancreatic acini, the TRPV4 channel agonist GSK10167790A (GSK101) and TRPV4 receptor antagonists HC067047 (HC067) and RN1734 were used. GSK101 (50 nM) induced a significant increase in [Ca²⁺]ᵢ, which was inhibited by both HC067 (100 nM) and RN1734 (30 μM) (FIG. 10C-D).

To determine the mechanism for TRPV4 activation, the PLA2 pathway was examined by first testing the endogenous AA metabolite ligand 5′,6′-EET. Similar to the TRPV4 agonist GSK101, AA and 5′,6′-EET induced significant increases in [Ca²⁺]ᵢ (FIG. 10E-F) that were inhibited by the TRPV4 antagonist HC067 (FIG. 10E-F). Next, it was determined whether the ability of shear stress to produce the sustained elevation in [Ca²⁺]ᵢ was due to TRPV4 by measuring [Ca²⁺]ᵢ following shear stress (12 dyne/cm² for 30 seconds) in the presence of the TRPV4 antagonist HC067. HC067 (1 μM, the concentration used to completely inhibit TRPV4 activity) blocked the sustained [Ca²⁺]ᵢ elevation, leaving only transient alterations in [Ca²⁺]ᵢ (FIG. 10G-H). Like shear stress experiments, HC067 completely blocked the Yoda1-stimulated (25 μM) sustained increase in [Ca²⁺]ᵢ (FIGS. 10I-J). Importantly, neither Yoda1 nor fluid shear stress (12 dyne/cm² for 30 seconds) were found to cause a sustained increase [Ca²⁺]ᵢ in pancreatic acini isolated from TRPV4-KO mice (FIG. 10K-L). Together, these findings indicate that Piezo1 directly senses fluid shear stress and initiates calcium influx. However, the activation of TRPV4 is responsible for the secondary sustained influx of calcium resulting in the sustained elevation in [Ca²⁺]ᵢ.

### Piezo1 Elevates PLA2 Activity

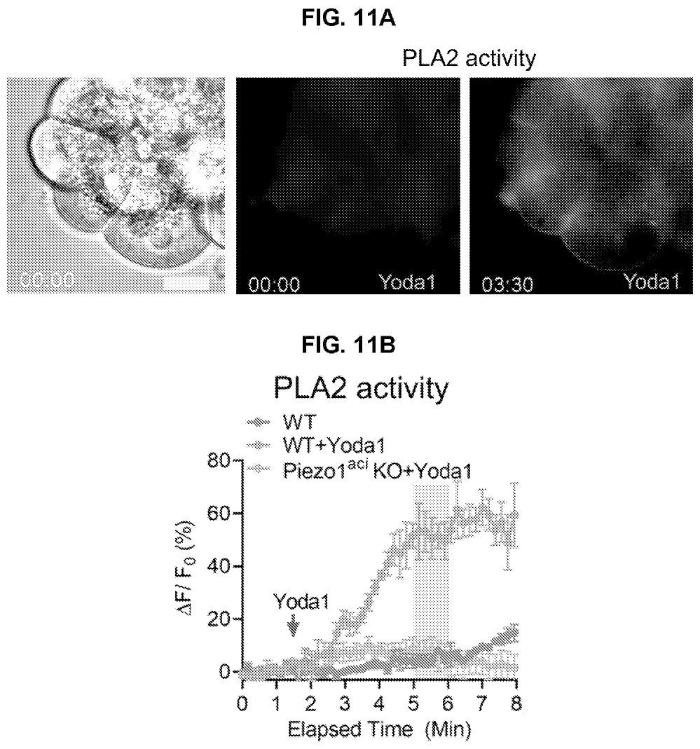

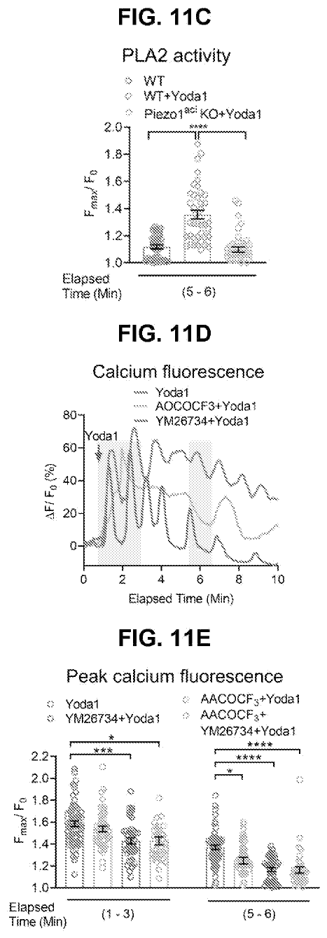

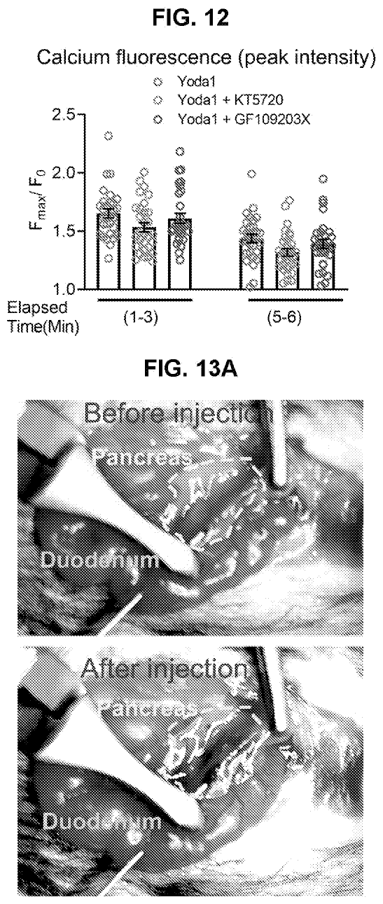

TRPV4 is activated by 5′,6′-EET generated through the PLA2-AA cytochrome P450 epoxygenase-dependent pathway. If this pathway is responsible for Piezo1-initiated TRPV4 channel activation, then Yoda1 should be able to induce PLA2 activation. To test this hypothesis, acinar cells were loaded with the fluorogenic PLA2 substrate 1, 2-Bis (4, 4-difluoro-5, 7-dimethyl-4-Bora-3a, 4a-diaza-s-indacene-3-undecanoyl)-Sn-glycero-3-phosphocholine (Bis-BODIPY FL C₁₁-PC). Prior to Yoda1 stimulation, initial Bis-BODIPY FL C₁₁-PC loaded pancreatic acini exhibited only faint fluorescence. However, application of Yoda1 markedly increased the intensity of the fluorogenic PLA2 substrate, indicating that Piezo1 is able to induce PLA2 activity (FIG. 11A-B). The effects of Yoda1 were observed within 30 seconds of application and reached a plateau after 4 minutes (FIG. 11B). Yoda1 did not stimulate PLA2 activation in pancreatic acini from Piezo1^{aci}-KO mice (FIG. 11-C). To confirm that the increase in PLA2 activity was responsible for the Piezo1-induced sustained [Ca²⁺]ᵢ levels, the secretory and cytosolic PLA2 inhibitors YM26734 and AACOCF3 were tested, respectively. Together, YM26734 and AACOCF3 blocked the Yoda1-induced sustained [Ca²⁺]ᵢ elevation, indicating that Piezo1 induced PLA2, which was responsible for the subsequent [Ca²⁺]ᵢ elevation (FIG. 11D-E). However, only the secretory PLA2 inhibitor effect was substantial and capable of blocking the sustained [Ca²⁺]ᵢ elevation (FIG. 11D-E). PKA and PKC inhibitors did not significantly affect Yoda-stimulated [Ca²⁺]ᵢ (FIG. 12).

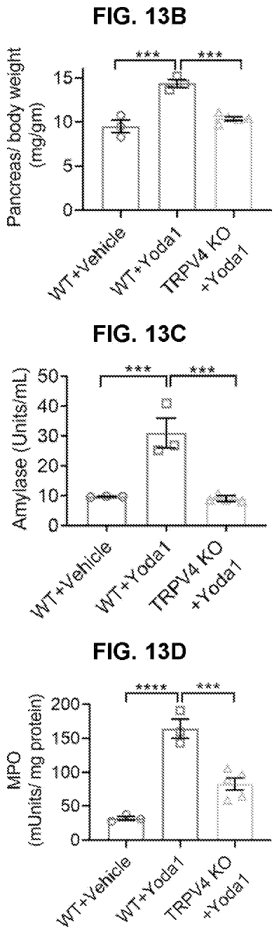

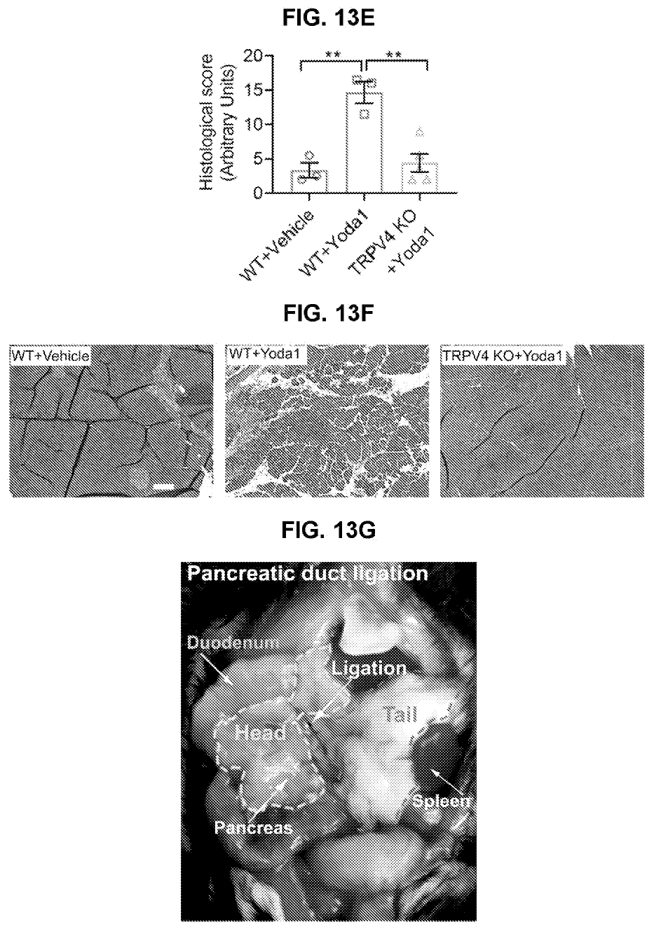

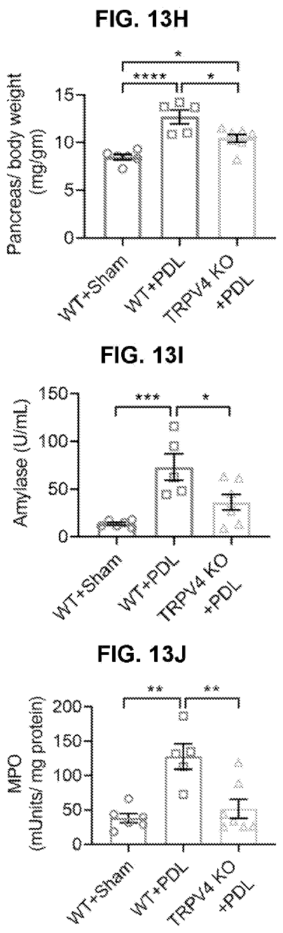

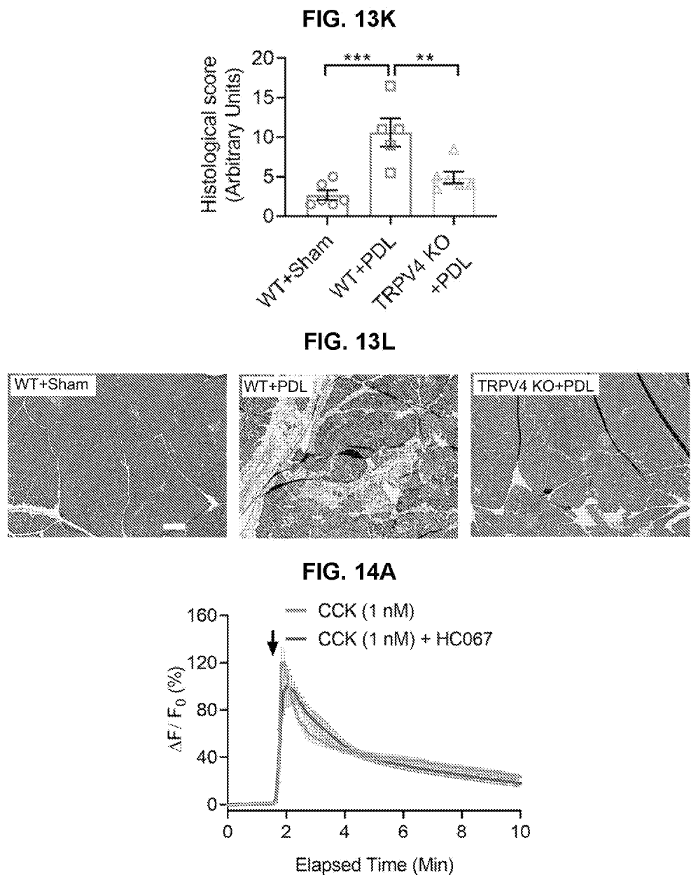

TRPV4-KO Mice are protected Against Pressure-Induced Pancreatitis Sustained elevations in [Ca²⁺]ᵢ are sufficient to cause pancreatitis. Having demonstrated that Piezo1-initiated, sustained [Ca²⁺]ᵢ elevation requires TRPV4 activation, it was proposed that TRPV4-KO mice could be protected from Piezo1-mediated pancreatitis. In order to selectively stimulate Piezo1 in the pancreas, Yoda1 (0.4 mg/kg) was infused into the pancreatic duct at a rate of 5 μL/min (FIG. 13A). This rate of infusion did not exceed the pancreatic duct pressure of 11 mmHg and is considered a low-pressure condition that alone does not cause pancreatitis. As shown in FIG. 13B-F, Yoda1 infusion increased all pancreatitis parameters measured (pancreatic edema, serum amylase, pancreatic myeloperoxidase [MPO], and histological scoring) in WT mice. In contrast, the same dose of Yoda1 did not cause pancreatitis in TRPV4-KO mice (FIG. 13B-F).

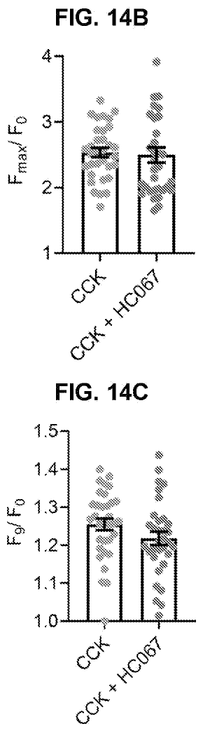

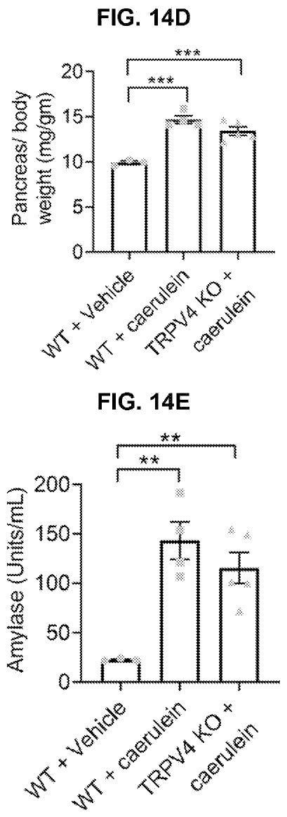

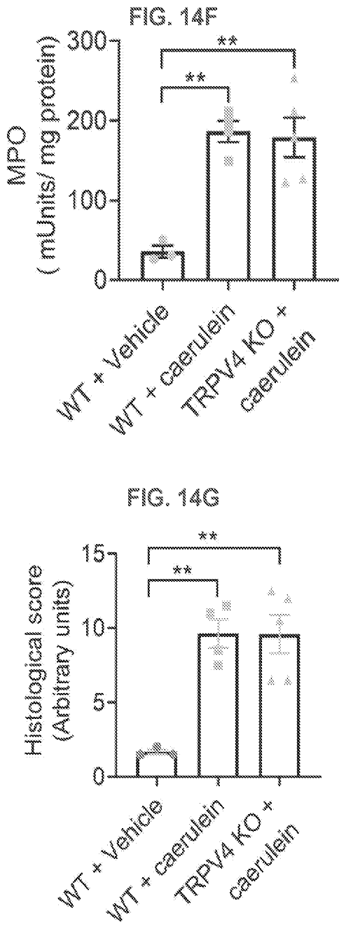

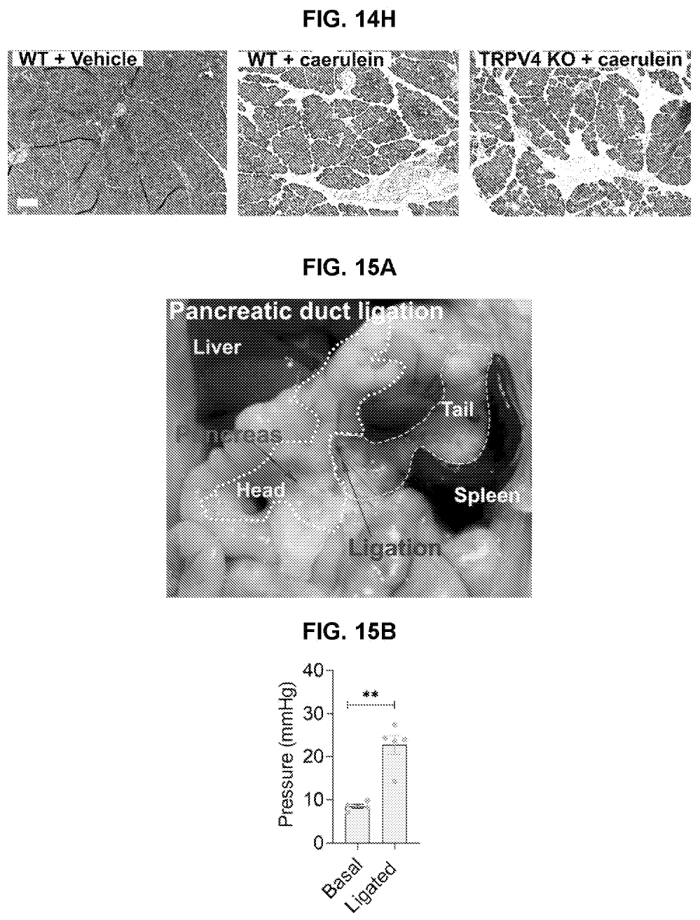

Having demonstrated that Piezo1 and CCK increase [Ca²⁺]ᵢ through separate mechanisms, it was proposed that CCK would not trigger TRPV4 channel opening. In support of this idea, it was found that the TRPV4 channel blocker HC067 did not affect the CCK-stimulated (1 nM) peak or sustained [Ca²⁺]ᵢ changes (FIG. 14A-C). It follows then that if CCK does not stimulate TRPV4 opening in acini, TRPV4-KO mice would not be protected from caerulein-induced pancreatitis. As expected, caerulein-induced pancreatitis was similar in both WT and TRPV4-KO mice (FIG. 14D-H).

Piezo1 is responsible for pressure-induced pancreatitis. Experimentally, Piezo1^{aci}-KO mice were protected against pancreatitis caused by high intrapancreatic duct pressure. If the pathological effects of Piezo1 activation are due to the downstream activation of TRPV4, it would be expected that mice lacking TRPV4 would be protected against pressure-induced pancreatitis. Pancreatitis was induced by ligating the tail region of the pancreas up to 24 hr (FIG. 13G). In WT mice, pancreatic duct ligation (PDL) caused acute tissue injury that was reflected by an increase in all pancreatitis parameters. In contrast, TRPV4-KO mice were protected against duct ligation-induced pancreatitis (FIG. 13H-L). These findings confirm that TRPV4 plays a key role in pressure-induced pancreatitis.

Acinar cells make up 90% of the pancreas and are notable for their abundant digestive enzymes that render the gland highly susceptible to pancreatitis should enzymes become prematurely activated following organ damage. Intracellular enzyme activation is a hallmark of pancreatitis and is believed to play a central role in disease pathogenesis. Recently, it was demonstrated that pressure activation of Piezo1 channels in pancreatic acinar cells is responsible for pressure-induced pancreatitis. Prior to the identification of Piezo1 in acinar cells, it was not known how the pancreas senses pressure, although a number of pathological situations indicate that the gland is exquisitely sensitive to pressure-induced injury. For example, intrapancreatic duct pressure is increased by occlusion of the pancreatic duct and this is thought to be a key factor in the development of gallstone-induced pancreatitis, which is one of the major causes of acute pancreatitis in humans. In this disease, impaction of a gallstone in the ampulla of Vater causes an abrupt increase in pancreatic duct pressure, leading to pancreatitis. In addition, mechanical force on the pancreas during trauma or pancreatic surgery may cause pancreatitis. Finally, injection of fluid into the pancreatic duct at high pressure during ERCP is a well-known cause of pancreatitis. In each of these causes of pancreatitis, abnormal pressure on the pancreas has the potential to activate Piezo1. However, Piezo1 is also expressed in other tissues, where it mediates several physiological processes, such as micturition, endothelial shear stress, embryogenesis, and vascular development. The question, therefore, arises of how activation of the Piezo1 channel leads to the pathological process of pancreatitis. In general, under normal physiological conditions, activation of Piezo1 channels is tightly regulated. Piezo1 channel function and transduction properties vary with stimulus frequency, waveform, and duration. It is proposed that overactivation of Piezo1 by persistent pressure or mechanical force in acinar cells produces downstream signaling events that disrupt normal cellular homeostasis, resulting in premature enzyme activation and ultimately pancreatitis. In the pancreas, disruption of calcium homeostasis is detrimental and leads to pancreatitis.

As disclosed herein, it is demonstrated that either chemical (Yoda1) or physical (shear stress) activation of Piezo1 induced [Ca²⁺]ᵢ overload, caused mitochondrial dysfunction, and led to intrapancreatic trypsinogen activation. Persistent application of the Piezo1 agonist Yoda1 at doses of 25 M and 50 μM produced a sustained increase in [Ca²⁺]ᵢ in pancreatic acini. A similar increase in [Ca²⁺]ᵢ was seen when cells were subjected to fluid shear stress of more than 12 dyne/cm² for 30 seconds. However, mechanical pushing of the acinar cell surface up to 5 μm for 1 second caused only a transient rise in [Ca²⁺]ᵢ and did not induce trypsinogen activation. Similarly, fluid shear stress at low pressure (4 dyne/cm²) or for a short duration (1 or 5 seconds) did not induce these changes. It has been previously established that a sustained elevation in [Ca²⁺]ᵢ in acinar cells is a prime cause of pancreatic injury. Elevated [Ca²⁺]ᵢ in pancreatic acinar cells produced by persistent Yoda1 exposure resembles that produced by supraphysiological doses of CCK, a well-known agent for inducing experimental pancreatitis. Thus, it seems reasonable to attribute the ability of Yoda1 to induce pancreatitis to its prolonged effects on [Ca²⁺]ᵢ. It is possible that brief push stimulation activates a subset of Piezo1 and chemical activation acts on all channels, which mimics the prolonged high shear stress-mediated pathological effects.

It was observed that removal of external Ca²⁺ abolished the Piezo1-mediated increase in [Ca²⁺]ᵢ and preincubating cells with the cell-permeable calcium chelator (BAPTA-AM) blocked the sustained increase in [Ca²⁺]ᵢ. Thus, it is possible that not only is external Ca²⁺ necessary for the Piezo1-mediated increase in [Ca²⁺]ᵢ, but also this increase is required for the opening of a Ca²⁺ entry pathway that contributes to the sustained elevation in [Ca²⁺]ᵢ. This is consistent with the observation that preincubating cells with BAPTA-AM protected acini from Piezo1-mediated cellular injury. Notably, Piezo1-mediated stimulation of [Ca²⁺]ᵢ differs from stimulation through the CCK-mediated pathway. The Piezo1 pathway may require external Ca²⁺ to initiate the process, whereas CCK stimulation begins via the release of Ca²⁺ from intracellular ER stores.

Under physiological conditions, the pancreatic secretagogues acetylcholine and CCK stimulate [Ca²⁺]ᵢ in a spatiotemporal manner that is required for ATP production, initiation of exocytosis, and nuclear signaling processes. However, supraphysiological CCK stimulation and excess ethanol, bile, and toxins induce a sustained elevation in [Ca²⁺]ᵢ in pancreatic acinar cells that cause acute pancreatitis. This sustained elevation in [Ca²⁺]ᵢ mediates mitochondrial dysfunction, premature zymogen activation, vacuolization, and necrosis. It was observed that both Yoda1 and fluid shear stress induced mitochondrial depolarization and trypsin activation in pancreatic acinar cells. Transient depolarization of mitochondria following a rise in [Ca²⁺]ᵢ is associated with normal cellular ATP production. However, [Ca²⁺]ᵢ overload in acinar cells induced by bile and ethanol opens the MPTP, collapses the mitochondrial membrane potential (ψm) required for ATP synthesis, and ultimately results in cell death. By chelating intracellular-free Ca²⁺, Yoda1-induced mitochondrial depolarization and cell death was prevented. It has been observed previously that intracellular calcium chelation prevents zymogen activation and protects against acute pancreatitis in vivo. It was observed that Piezo1-mediated mitochondrial depolarization preceded trypsin activation following either Yoda1 treatment or shear stress and is ultimately responsible for pressure-induced pancreatitis.

Piezo1 is a fast-inactivating channel with single-channel conductance of approximately 22 pS (inward current), which is lower than the TRPV4 ion channel of approximately 60 pS. Piezo1 inactivation kinetics are independent of stimulus intensity. If no other type of channel is present in the cell except Piezo1, the calcium rise will be transient rather than sustained due to fast inactivation kinetics. This suggests that the Piezo1-induced sustained elevation in [Ca²⁺]ᵢ produced by Yoda1 or shear stress requires an extra calcium entry pathway. Therefore, other potential channels were sought that could be linked to Piezo1 activity, and it was discovered that TRPV4 is expressed in both mouse and human pancreatic acini.

Initially, it was thought that mechanical activation of Piezo1 and TRPV4 were independent processes. Even though activation of Piezo1 could produce a transient increase in [Ca²⁺]ᵢ and TRPV4 could produce more sustained elevation of [Ca²⁺]ᵢ by virtue of its slow inactivation kinetics and considerably higher single channel conductance, it was not clear whether the two processes were linked. Remarkably, the TRPV4 antagonist HC067 completely blocked the sustained phase of calcium elevation induced by Yoda1 and shear stress. This provided the hint that Piezo1 regulates TRPV4 channel activation. The results were confirmed in experiments from TRPV4-KO mice when both Yoda1 and prolonged shear stress produced only transient elevation in [Ca²⁺]ᵢ. Low shear stress for 30 seconds and high shear stress for 5 seconds were not sufficient to induce a sustained calcium rise and did not activate TRPV4. The reason could be that the brief force caused only a subset of Piezo1 channel openings and was insufficient to activate PLA2. In the cell, PLA2 is activated upon binding to calcium ions that accelerate enzyme activity, which initiates the arachidonic pathway and TRPV4 channel activation. In mouse pancreatic acinar cells, the Piezo1 agonist Yoda1 increased PLA2 activity and caused a sustained elevation in [Ca²⁺]ᵢ, an effect that was inhibited by a PLA2 inhibitor. Various reports have indicated that TRPV4 is sensitive to mechanical stimuli, such as osmotic pressure, shear stress, and mechanical stretching. However, it was unclear how mechanical stimuli actually activate the TRPV4 channel. The current findings indicate that Piezo1 stimulation of PLA2 and subsequent activation of TRPV4 could be a mechanism by which TRPV4 channels respond to mechanical force.

Supramaximal doses of CCK secretagogue block apical secretion and cause intracellular vacuolization, enzyme activation, and enzyme release from the basolateral surface of the cell. It was observed that Yoda1 induced the release of vesicles from the basolateral surface, indicating a possible pathological situation.

No significant effects of PKA and PKC inhibitors on Yoda1-mediated TRPV4 activation were observed. In other cells, such as HEK293 cells or human coronary artery endothelial cells, PKA and PKC can directly phosphorylate TRPV4 and modify channel activity. These findings suggest that this pathway is not required for channel activation.

These results demonstrate that Piezo1 initiates the pressure-induced calcium signal, causing TRPV4 activation, but the pathological events that occur in the acinar cell may require TRPV4-mediated calcium influx, which is responsible for the sustained phase of calcium elevation that leads to pancreatitis. Consistent with this pathological sequence of events, TRPV4-KO mice were protected from Yoda1-induced pancreatitis.

To mimic pancreatitis caused by pancreatic duct obstruction (e.g., gallstones), a mouse model of pancreatitis was used by ligating the tail region of the pancreas for 24 hr. In this model, it was observed that TRPV4-KO mice were substantially protected. Hence, a TRPV4 channel blocker could be a possible treatment for pancreatitis where pressure is encountered.

These findings suggest that activation of Piezo1 in the absence of TRPV4 is not sufficient for inducing pathological calcium signaling. However, when coexpressed and linked by intracellular signaling pathways, TRPV4 may appear to be pressure sensitive. For example, Piezo1 and TRPV4 channels are expressed in endothelial cells, and previous reports have indicated that high pulmonary venous pressure induces Ca²⁺ influx into endothelial cells via TRPV4 channels, resulting in increased vascular permeability, which is a major cause of mortality in heart failure patients. Although unrecognized at the time, this process may be linked to Piezo1, which appeared to sense high vascular pressures at the lung endothelial surface and account for vascular hyperpermeability and pulmonary edema. Thus, it appears that both Piezo1 and TRPV4 are responsible for this vascular hyperpermeability, and these findings suggest that they may be linked. It is hypothesized that Piezo1 sensing of high vascular pressure initiates a Ca²⁺-signaling pathway that triggers the activation of TRPV4. TRPV4 activation would then cause a secondary, sustained Ca²⁺ influx that would lead to vascular hyperpermeability. The extent of TRPV4-induced Ca²⁺ entry would be influenced by other factors, including the level of TRPV4 expression, the degree and duration of pressure, and, if identical to the pancreas, the appropriate level of PLA2 activity. Thus, these findings may represent a more generalized process in which TRPV4 converts Piezo1 pressure sensing into a pathological event.

### Example 3

### Materials and Methods

### Animals

To generate Piezo1 deletion in stellate cells, Piezo1^{fl/fl} mice were crossed with B6.Cg-Tg(GFAP-cre/ERT2)505Fmv/J mice (The Jackson Laboratory) to generate the mouse line B6.Cg-Tg(GFAP-cre/ERT2); piezo1^{fl/fl}. To generate conditional genetic Piezo1 deletion in stellate cells, 40 mg of tamoxifen/kg body weight (Millipore Sigma, T5648) was injected i.p. per day for 5 consecutive days. The mice were used 8 days after the last tamoxifen injection. The mouse lines B6.Cg-Tg (GFAP-cre/ERT2); piezo1^{fl/fl} and ptf1a^{CreERTM}; piezo1^{fl/fl} after tamoxifen injection were referred to as Piezo1^{GFAP}-KO and Piezo1^{aci}-KO, respectively. Piezo1^{fl/fl} mice were used as WT in the experiments with Piezo1^{aci}-KO and Piezo1^{GFAP}-KO mice. Seven- to 12-week-old mice (both male and female) were used in the experiments. A small piece of tail of each mouse was used for genotyping. The mouse line with Trpv4 gene deletion was referred to as TRPV4-KO. C57BL/6J mice (The Jackson Laboratory) were used as WT mice in the experiments with TRPV4-KO mice. Mice were housed under standard 12-hour light/12-hour dark conditions.

### In Vivo Experiments

PDL of the tail region was performed as previously described by Swain et al., J. Clin. Invest. 130(5): 2527-2541 (2020). The pancreas was visualized using a stereomicroscope, and the tail region of the main pancreatic duct was ligated with 7-0 (0.5 metric) nonabsorbable, Prolene suture without damaging underlying arteries and veins. Mice suffering injury to any underlying blood vessels were excluded from the experiment. Mice were sacrificed at day 8 or day 30 after surgery.

### In Vitro Experiments

Mouse and human PSCs were isolated using collagenase digestion. Modified Krebs Henseleit Buffer (KHB) solution (100 mL) was prepared as previously described by Romac et al., Nat. Commun. 9(1):1715 (2018). Pancreatic tissue was digested with 2 mg of collagenase NB 8 (SERVA, catalog 17456) dissolved in 10 mL of modified KHB solution containing 1 mg soybean trypsin inhibitor (SBTI 1-S; Millipore Sigma, catalog T9003) and 20 mg BSA (Thermo Fisher Scientific; BP1600-100). Digestion solution (5 mL) was used to inflate the pancreas. Pancreas with 5 mL of digestion solution was incubated in a shaking water bath for 10 minutes at 37° C. The solution was then discarded. The pancreas tissue was cut into small pieces and digested with fresh 5 mL digestion buffer. Tissue was incubated in a shaking water bath for 40 minutes at 37° C. The cells were then separated from tissue by pipetting up-down with a 10 mL pipette and passed sequentially through a 70 μm and a 40 μm cell strainer. The filtrate was centrifuged at 150×g for 3 minutes at room temperature. The cells were washed with 10 mL Leibovitz's media (Thermo Fisher Scientific, catalog 11415-064), passed through a 20 μm filter, and centrifuged at 80×g for 3 minutes at room temperature. The supernate was removed, and isolated stellate cells were plated on a thin-layered Matrigel-coated glass bottom culture plate (MatTek, P35G-0-14-C). Before plating the cells, Matrigel solution (Corning, catalog 354234) at a ratio of 1.5:100 DMEM/F12 (Thermo Fisher Scientific, catalog 11330-032) was poured onto the culture plate and incubated for 2 hr at 37° C. to form a thin layer of Matrigel coating. Matrigel mixture was removed, and the plate was washed with PBS before cells were plated. Cell culture media, DMEM/F12 with 5% FBS was used for mouse stellate cells. Fresh, human pancreatic tissue was digested with collagenase as described above with modifications. Collagenase (2.5 mg) in 10 mL of modified KHB solution was used for digestion. The digested tissues were filtered through a 100 μm cell strainer. The cells were cultured with DMEM/F12 with 10% FBS in a Matrigel-coated plate. After 24 hr, the cell media was replaced with fresh media to remove unattached and dead cells. After 2 days, cells were immunostained for GFAP and used for experiments. Perinuclear fat droplets were stained with BODIPY 493/503 (4,4-Difluoro-1,3,5,7,8-Pentamethyl-4-Bora-3a,4a-Diaza-s-Indacene; Invitrogen, catalog D3922) to confirm stellate cell quiescence. The cell viability following Yoda1 treatment was analyzed using the Live/Dead cell imaging kit (Thermo Fisher Scientific, catalog R37601). RNAs were isolated using the RiboPure Kit (Invitrogen, catalog AM1924) according to manufacturer's instructions.

### Shear Stress and Mechanical Pushing

Fluid shear stress and mechanical pushing applied to stellate cells were achieved as previously described by Wang et al., J. Clin. Invest. 126(12): 4527-4536 (2016). Parallel-plate fluid flow chambers (μ-Slide I 0.4 Luer, or μ-Slide I 0.2 Luer from Ibidi) were used for fluid shear stress experiments. The fluid shear stress (T) was calculated using the formula: τ=η×104.7.6 φ for μ-Slide I 0.4 Luer and τ=η×330.4 φ for μ-Slide I 0.2 Luer, where r represents viscosity of the medium and φ represents flow rate (according to the manufacturer's instructions, Ibidi). Mechanical pushing was achieved by applying a blunt borosilicate glass pipette once to the cell membrane with a deflection of 2-3 μm for 1 second using a micromanipulator (World Precision Instruments).

### Immunostaining

Mouse and human PSCs were washed with PBS (pH 7.4) and fixed with 4% paraformaldehyde for 10 minutes at room temperature. The fixed cells were treated with 0.1% Triton X-100. Cells were immunostained with a rabbit anti-TRPV4 antiserum (Alomone, ACC-034, 1:250, rabbit anti-Piezo1 antiserum (Alomone, APC-087, 1:300), rabbit anti-fibronectin antibody (Abcam, ab2413), rabbit anti-collagen type I antibody (Abcam, ab34710), or chicken anti-GFAP antibody (Abcam, 4674) for mouse PSCs or rabbit anti-GFAP (Cell Signaling Technology, 12389) for human PSCs overnight at 2-8° C. Secondary antibodies included DyLight 488-conjugated anti-chicken IgG (Jackson ImmunoResearch, 703-546-155), DyLight 488-conjugated anti-rabbit IgG (Jackson ImmunoResearch, 711-546-152), or CY3-conjugated anti-mouse IgG (Jackson ImmunoResearch, 715-166-150), used for 1 hr at room temperature. Nuclei were stained with Nunc blue (Invitrogen, R37606). All staining images were taken with a Zeiss Axio observer Z1 with a 20× or 40× objective.

### Ca²⁺ Imaging

Calcium 6-QF (Molecular Devices) dye was used for live-cell calcium imaging as described by Romac et al., Nat. Commun. 9(1):1715 (2018). Cells were imaged in HBSS buffer with 2 mM Ca²⁺. Faintly and highly fluorescent loaded cells were excluded from analysis. The chemicals used in calcium imaging experiments included: GsMTx4 (Abcam, catalog ab141871), Yoda1 (Tocris, catalog 5586), GSK1016790A (Millipore Sigma, catalog G0798), HC067047 (Tocris, catalog 4100), YM26734 (Tocris, catalog 2522), and AACOCF3 (Tocris, catalog 1462).

### Histologic Grading

Head and tail regions of the pancreas were embedded in paraffin and sectioned at 5 μm thick. The tissue sections were stained with Masson's trichrome or H&E. The images were captured on an EVOS microscope using an Olympus PlanApo 10× objective. The histological scores for chronic pancreatitis severity were calculated as described by Van Laethem et al., Gastroenterology 110(2): 576-582 (1996). The severity of chronic pancreatitis was graded by considering 5 pathological parameters (inflammatory infiltrate, atrophy, intralobular fibrosis, perilobular fibrosis, and interlobular fibrosis), and each category was scored on a scale of 0 (no injury) to 3 (maximum injury). Total scoring was presented after adding all pathologic parameter scores and was on a scale of 0 (no injury) to 15 (maximum injury). The degree of fibrosis was measured by quantifying the areas stained with Masson's trichrome.

### Statistics

Data were analyzed using GraphPad Prism 9. Results were represented as mean±SEM. Two-tailed Student's t test was used for 2-group comparisons, and 1-way ANOVA with Tukey's multiple-comparison test was used for multigroup data sets. P<0.05 was considered significant. *P≤0.05; **P≤0.01; ***P≤0.001; ****P≤0.0001.

### Example 4

### Increased Intrapancreatic Pressure Causes Pancreatic Fibrosis

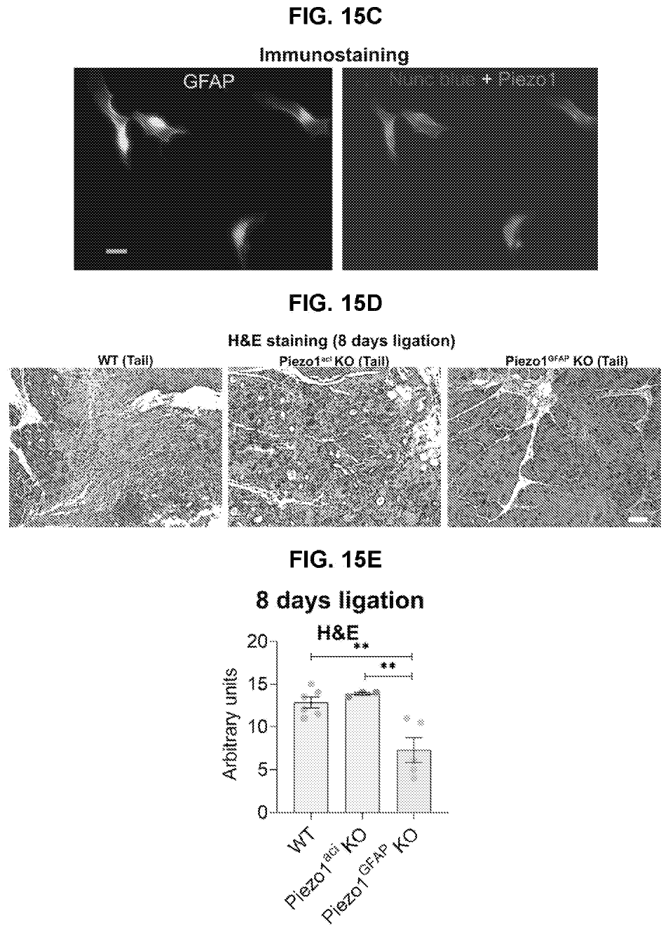

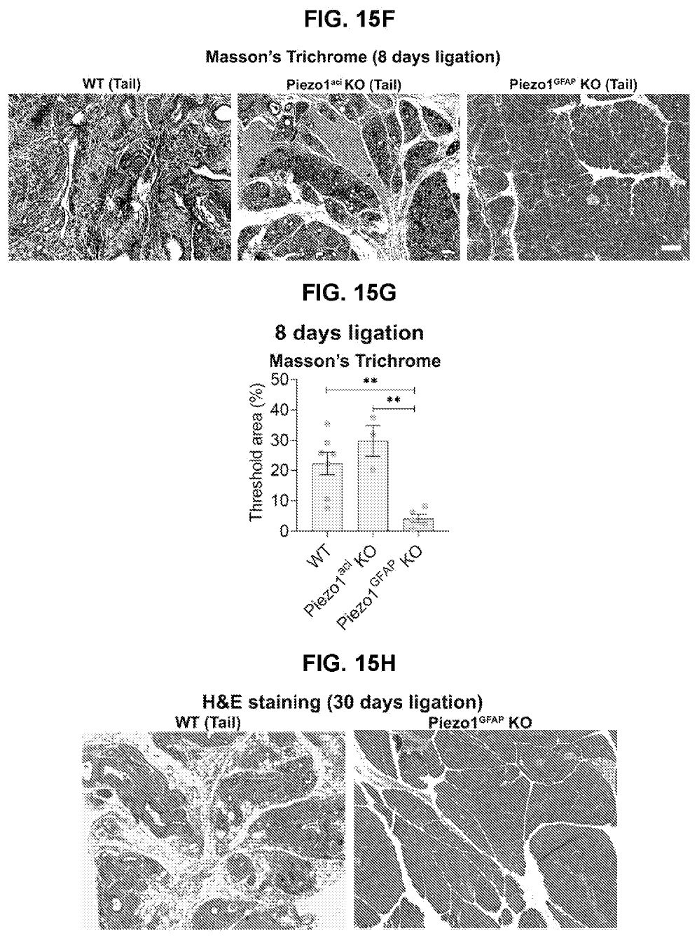

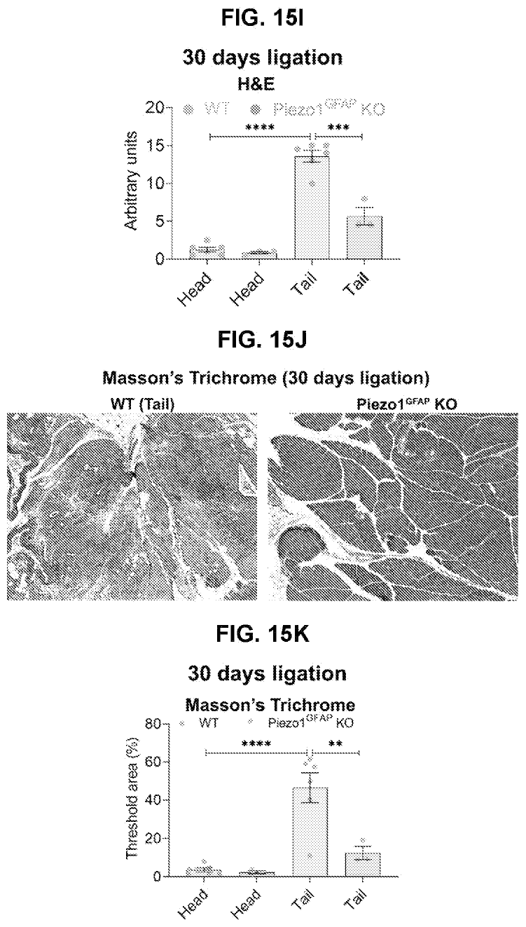

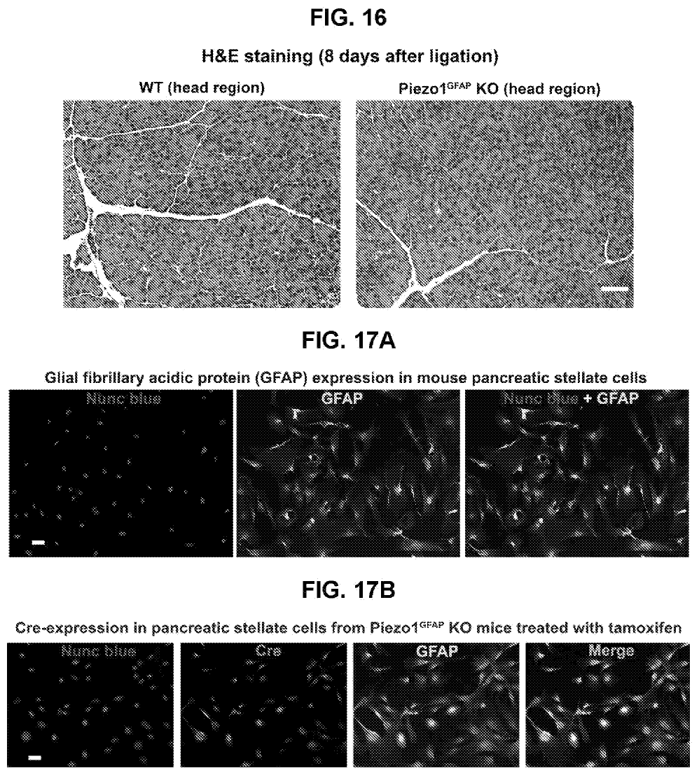

A clinically relevant obstructive pancreatitis model was developed by ligating the pancreatic tail region (FIG. 15A) to evaluate the effects of intrapancreatic pressure on fibrosis. Five minutes after ligation, pancreatic pressure in the tail region increased from 8.5 mmHg (unligated pressure) to 22.7 mmHg (ligated pressure) (FIG. 15B). Eight days after pancreatic duct ligation (PDL), substantial pancreatic fibrosis was observed in the tail region of the pancreata. The nonligated head region of the pancreas was unaffected (FIGS. 15D-G and 15J, and FIG. 16). Similar results were observed 30 days after duct ligation (FIG. 15H-K). Accompanying the high deposition of collagen was loss of endocrine and exocrine tissue in the tail region (FIG. 15J-K).

Piezo1^{GFAP}-KO Mice are Protected from Pressure-Induced Pancreatic Fibrosis

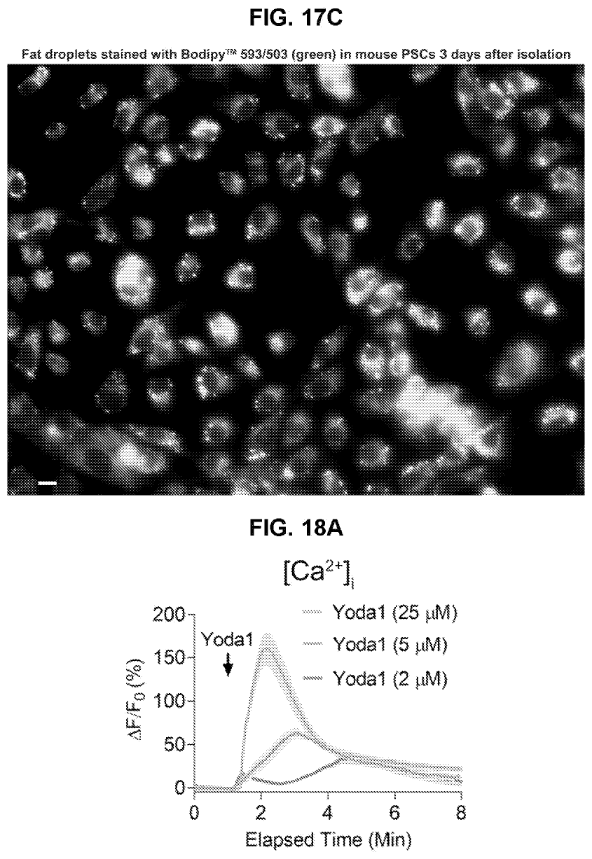

The demonstration that duct obstruction induced pancreatic fibrosis raised the possibility that PSCs were pressure sensitive. Mechanically activated ion channels are key pressure sensors in many tissues, and it was previously demonstrated that the most highly expressed mechanoreceptor in the pancreas is Piezo1. To determine possible mechanoreceptor expression, Piezo1 was identified in PSCs by immunostaining (FIG. 15C and FIG. 17A-B). To evaluate the potential role of Piezo1 in PSCs and pancreatic fibrosis, Piezo1 was deleted in cells expressing the stellate cell gene glial fibrillary acidic protein (GFAP). Piezo1^{GFAP}-KO mice were protected from partial duct ligation-induced pancreatic fibrosis 8 days after ligation (FIG. 15D-G). In contrast, mice with selective deletion of Piezo1 in pancreatic acinar cells (Piezo1^{aci}-KO mice) were not protected against pressure-induced fibrosis (FIG. 15D-G). Similarly, 30 days after duct ligation, substantial collagen deposition with loss of pancreatic exocrine and endocrine tissue was observed in WT but not in Piezo1^{GFAP}-KO mice (FIG. 15H-K). These findings indicate that pressure induced by duct ligation induces pancreatic fibrosis by activating stellate cells independently of acinar cell-mediated pancreatitis.

Piezo1 Causes Sustained [Ca²⁺]ᵢ Elevation in PSCs

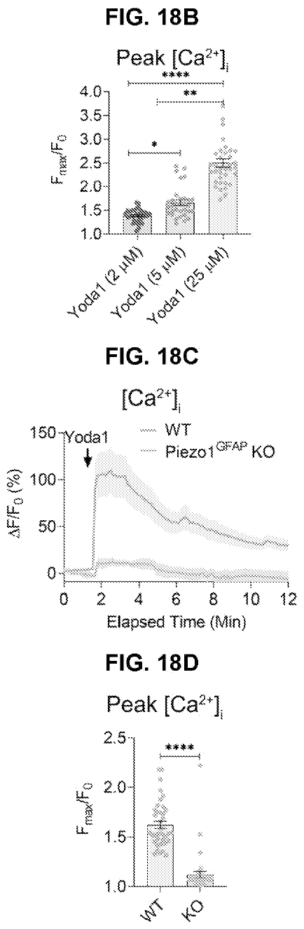

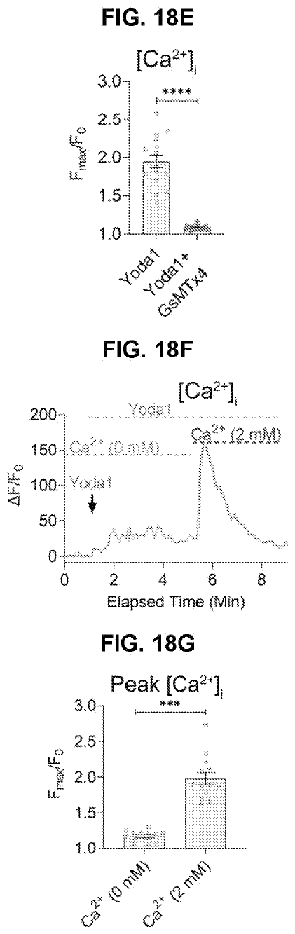

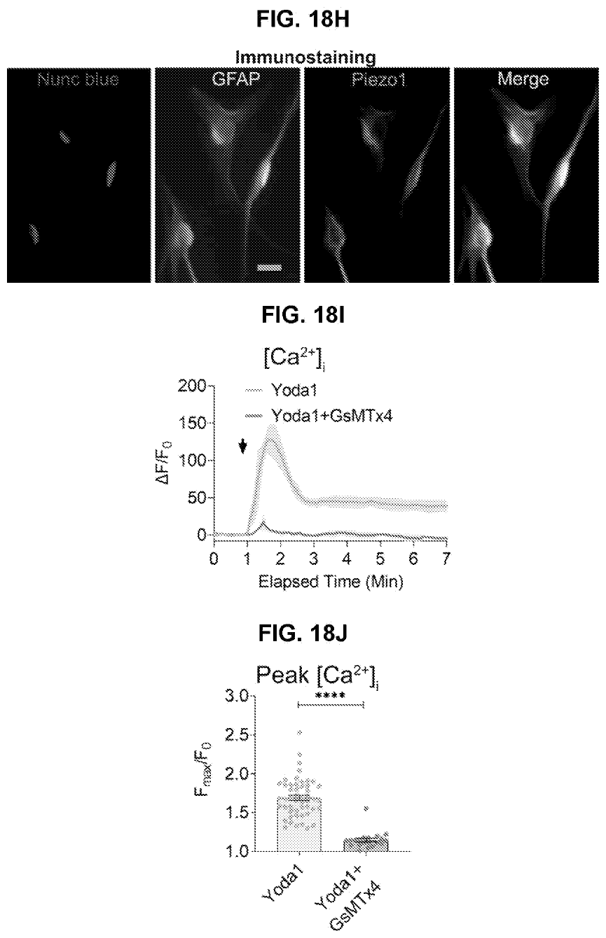

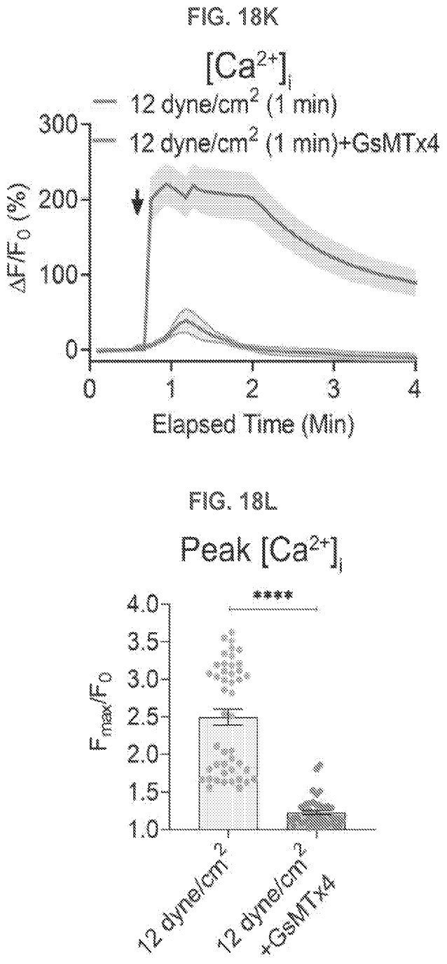

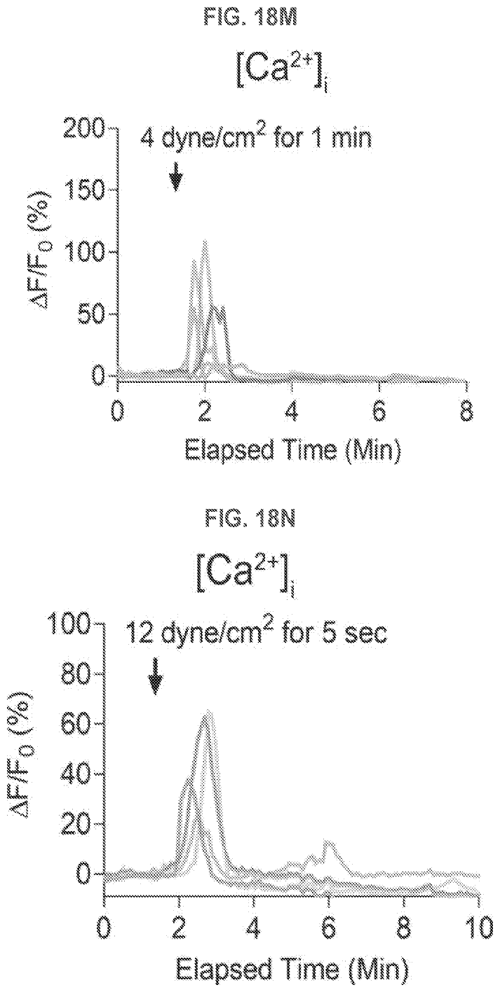

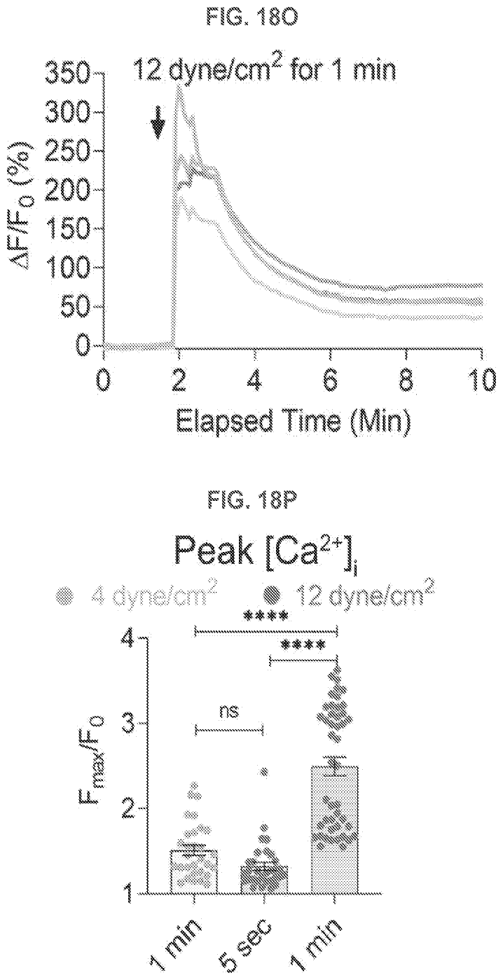

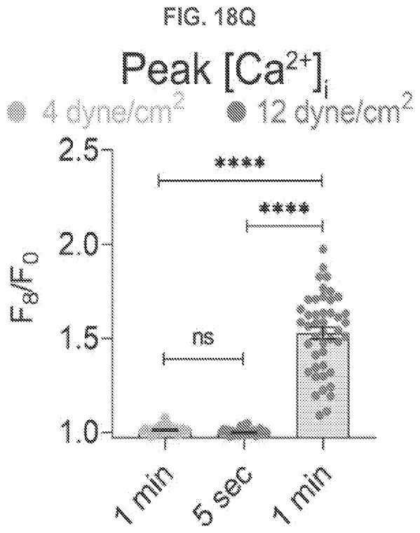

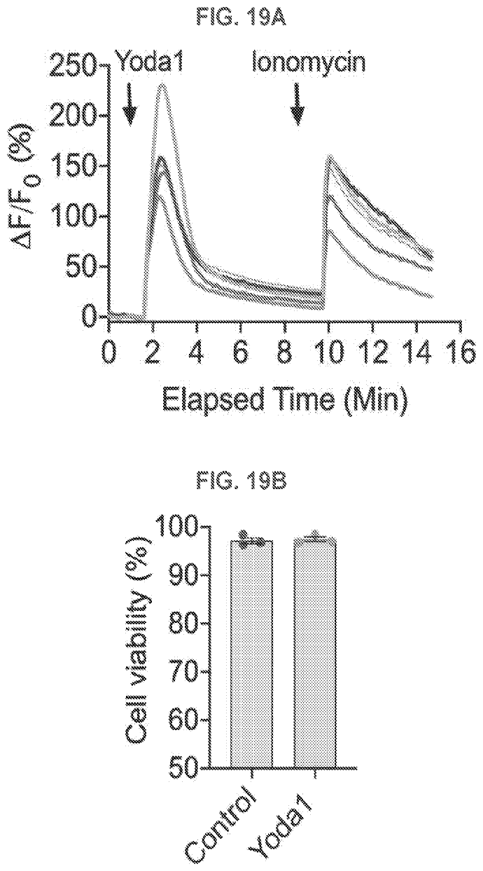

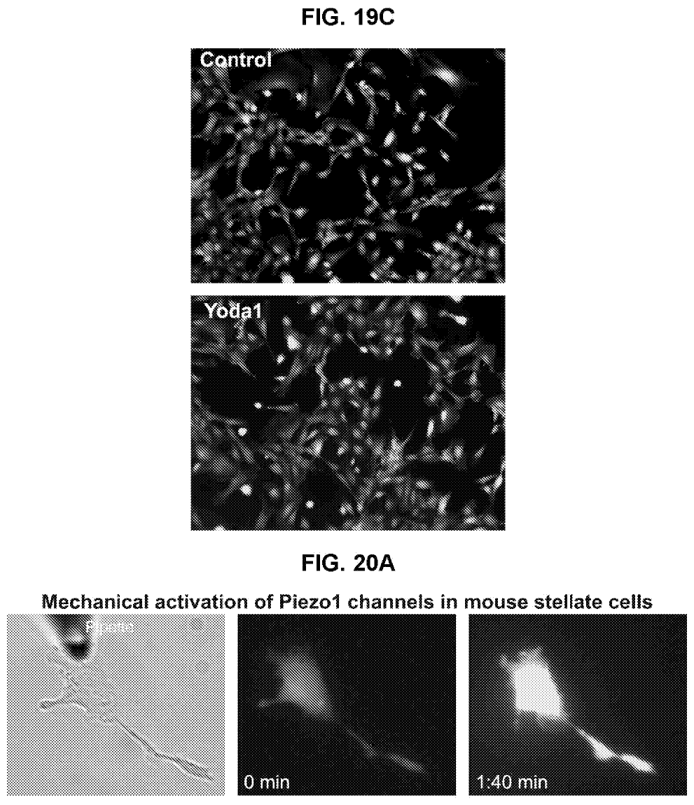

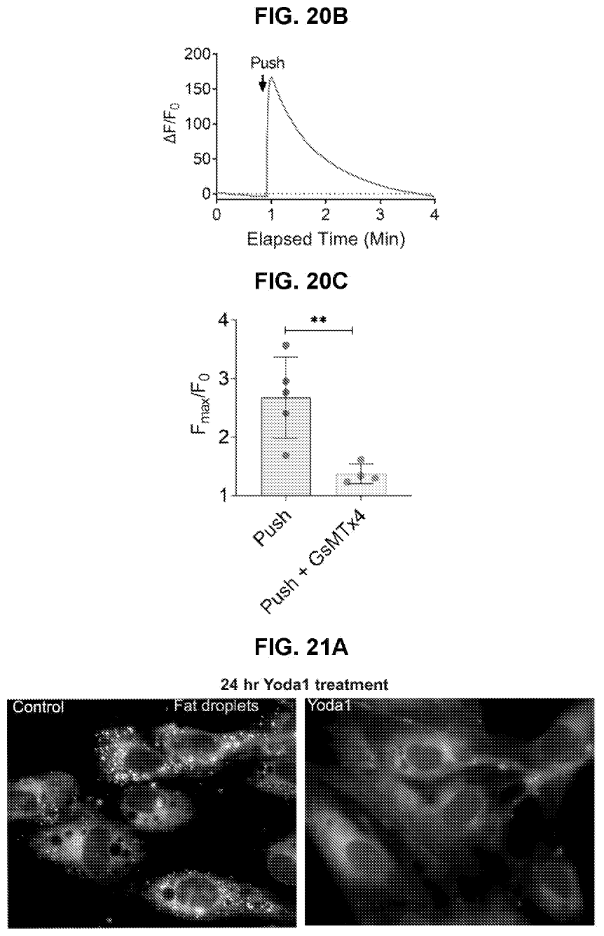

PSCs are known to produce excessive ECM proteins in pancreatic fibrosis, so it was hypothesized that increased intrapancreatic pressure stimulates increased PSC ECM production. To investigate this possibility, mouse PSCs were first cultured on a Matrigel-coated plate to maintain a quiescent phenotype. As evidence of quiescence, nearly 97% of PSCs retained Bodipy+perinuclear fat droplets (FIG. 17C). To determine possible mechanoreceptor expression, Piezo1 was identified in PSCs by immunostaining (FIG. 15C and FIG. 17A-B), and functional activation was demonstrated by dose-dependent increases in [Ca²⁺]ᵢ elevation in response to the Piezo1 agonist Yoda1 (FIG. 18A-B). The Yoda1-stimulated increase in [Ca²⁺]ᵢ was absent in Piezo1-deleted PSCs (FIG. 18C-D), confirming that the effects of Yoda1 are specific for Piezo1. GsMTx4, a Piezo1 antagonist, blocked the calcium rise produced by Yoda1 (FIG. 18E). Removing external calcium abolished the Yoda1-induced calcium influx and replacing calcium (2 mM) restored calcium entry, demonstrating that the Piezo1-mediated increase in [Ca²⁺]ᵢ in PSCs was dependent on external calcium (FIG. 18F-G). High-dose Yoda1 (25 μM) did not affect PSC viability and membrane integrity. All cells responded to the calcium ionophore, ionomycin, after Yoda1 (25 μM) treatment (FIG. 19). Like mouse PSCs, Piezo1 channels are expressed in human PSCs and are sensitive to GsMTx4 blockade following Yoda1 (25 μM) stimulation (FIG. 18H-J). Mechanical forces such as shear stress and pressure are physiological activators of Piezo1. Mechanical force was applied by touching PSCs with a glass pipette. Pressure applied for 1 second produced only a transient rise in [Ca²⁺]ᵢ (FIG. 20). To apply mechanical force for longer periods of time and to avoid cell accommodations to prolonged mechanical pushing, fluid shear stress was used. Prolonged high fluid shear stress is an approach similar to high-fluid pressure situations during obstructive pancreatitis. To determine the effects of shear stress on human PSCs, fluid shear stress was applied at 12 dyne/cm² for 1 minute, which produced a sustained increase in [Ca²⁺]ᵢ similar to that of Yoda1 (25 μM). These changes were blocked by GsMTx4 (FIG. 18K-L). In contrast to the effects of high shear force, low shear stress (4 dyne/cm² for 1 minute) or high shear stress of shorter duration (12 dyne/cm² for 5 seconds) produced only a transient rise in [Ca²⁺]ᵢ (FIG. 18M-Q). These findings indicate that the effects of mechanical force on [Ca²⁺]ᵢ are dependent on force and time.

### Piezo1 Triggers PSC Activation

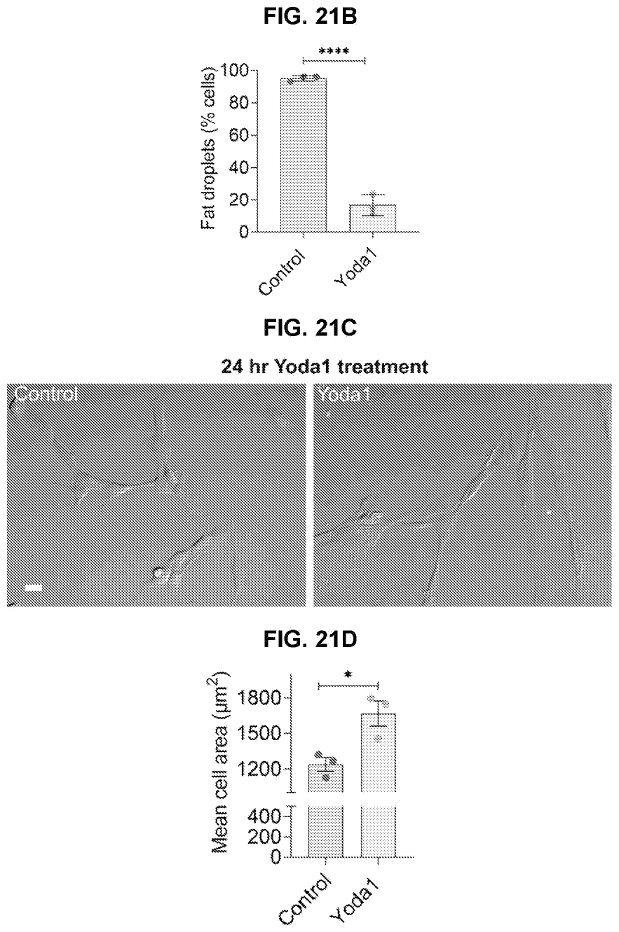

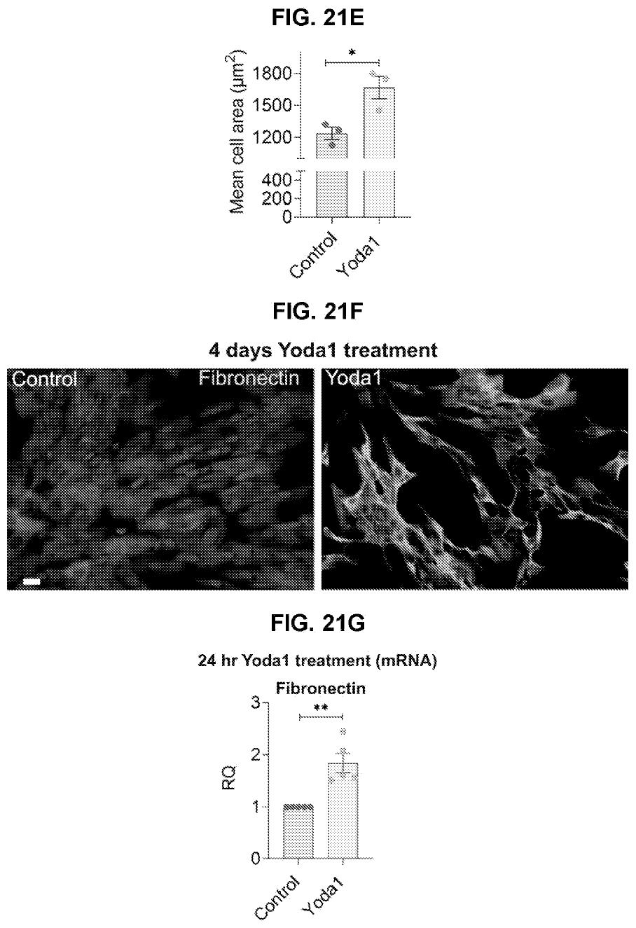

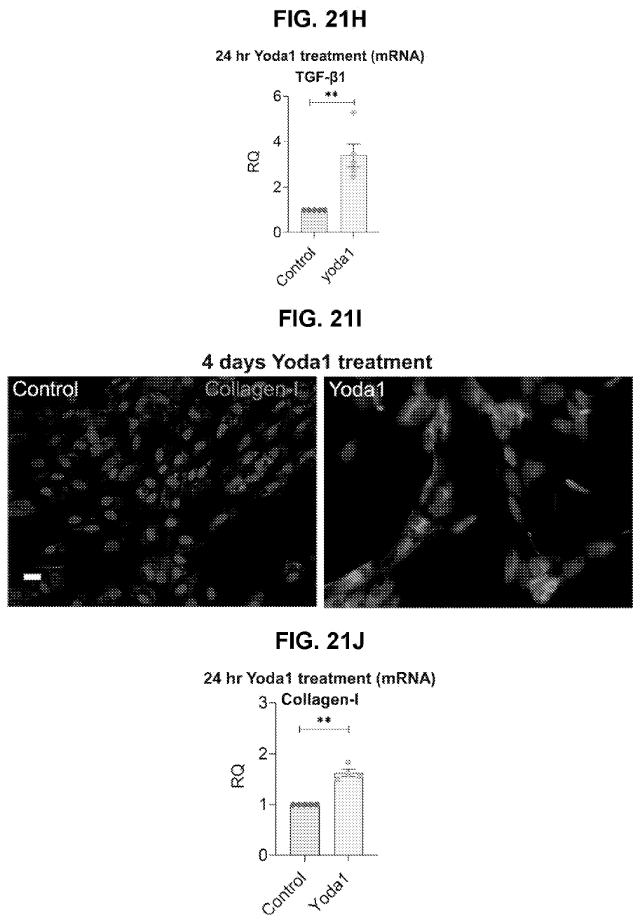

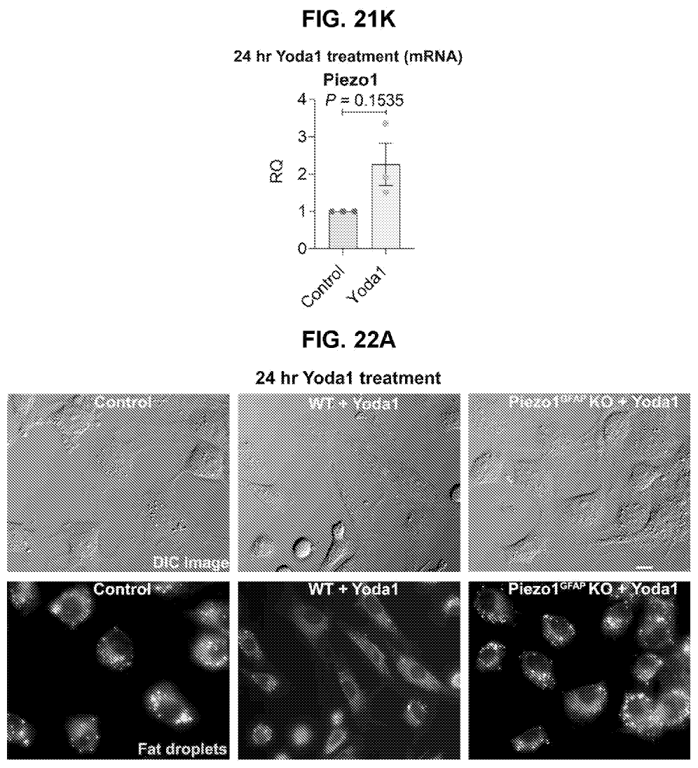

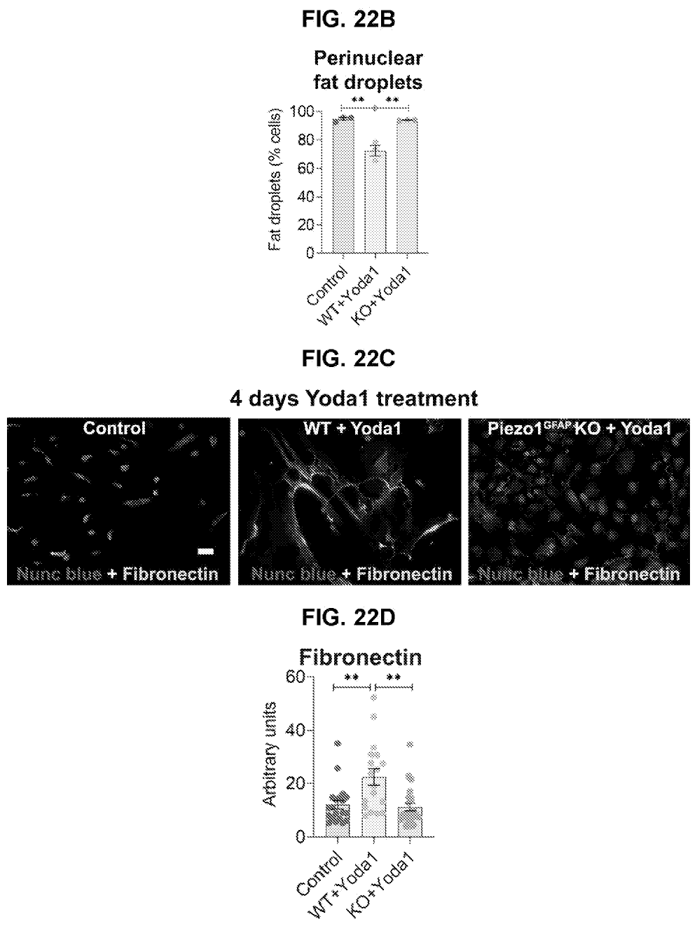

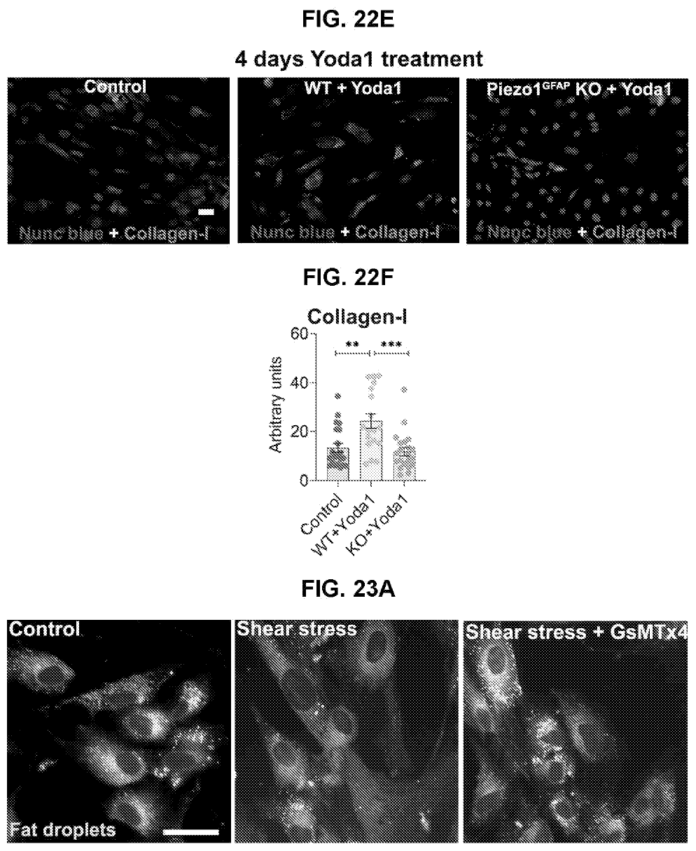

In healthy pancreas, quiescent PSCs are characterized by perinuclear, vitamin A-containing fat droplets and low-level expression of ECM proteins such as fibronectin and collagen. Upon activation, PSCs lose perinuclear fat droplets and produce excess ECM proteins. To determine if Piezo1 has the ability to convert quiescent PSCs to an activated phenotype, Yoda1 (25 μM) was applied to human PSCs. Within 24 hours, nearly 80% of PSCs had lost their perinuclear fat droplets and became elongated with a significant increase in mean cell area and maximum diameter (mean Feret's diameter-max; FIG. 21A-E). Fibronectin, collagen type I, and Piezo1 mRNA levels were also significantly elevated (FIGS. 21G-H and 21J-K). After 4 days of Yoda1 treatment, fibronectin and collagen type I immunostaining were abundant (FIGS. 21F and 211). Like human PSCs, Yoda1 converted mouse PSCs to an activated phenotype with reduced perinuclear fat droplets, changes in cell shape, and increased fibronectin and collagen type I (FIG. 22). All the effects of Yoda1 on fibrosis were prevented in Piezo1-deleted PSCs (FIG. 22), confirming their Piezo1 dependence.

### High Shear Stress Induces PSC Activation and Fibrogenic Responses In Vitro

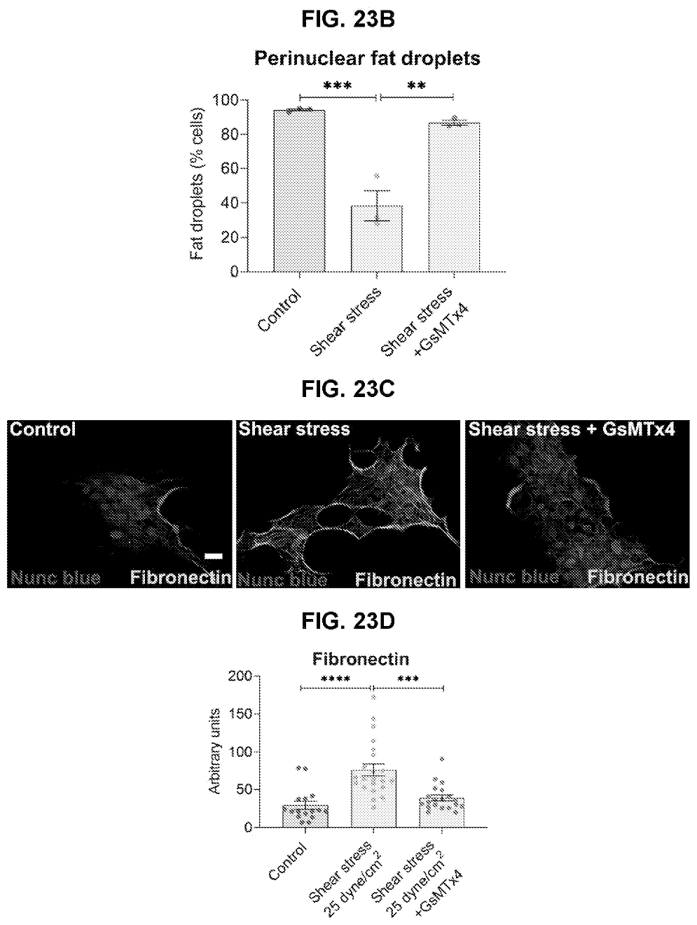

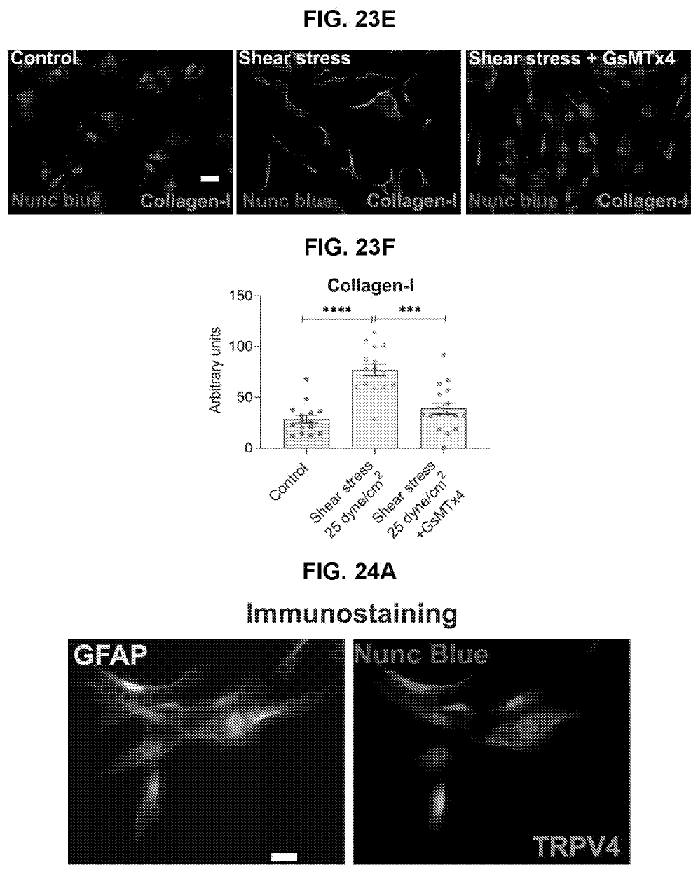

Application of high shear stress (12 dyne/cm²) for 1 minute produced sustained [Ca²⁺]ᵢ elevation in PSCs. To determine if physical force mediates stellate cell activation through Piezo1, the effect of high fluid shear stress was studied with and without the Piezo1 blocker, GsMTx4. Shear stress (12 dyne/cm² for 10 minutes) applied to human PSCs significantly reduced the number of perinuclear fat droplets (FIG. 23A-B). Repeated injury to the pancreas, associated with edema and increased pancreatic pressure, leads to chronic pancreatitis. The observation that Yoda1 increased Piezo1 expression (FIG. 21K) raised the possibility that repeated exposure to mechanical force may induce a Piezo1-mediated fibrogenic response. To test this hypothesis, shear stress (25 dyne/cm² for 10 minutes) was applied twice at an interval of 24 hr and the stellate cell activation and fibrogenic responses were examined in vitro. Importantly, repeated shear stress increased fibronectin and collagen type I in human PSCs (FIG. 23C-F). Treatment with the Piezo1 blocker, GsMTx4, attenuated the shear stress-mediated pathological changes (FIG. 23), demonstrating that mechanical force induced stellate cell activation and increased ECM protein synthesis through Piezo1.

### Piezo1 Signaling Mediates TRPV4 Channel Opening in PSCs

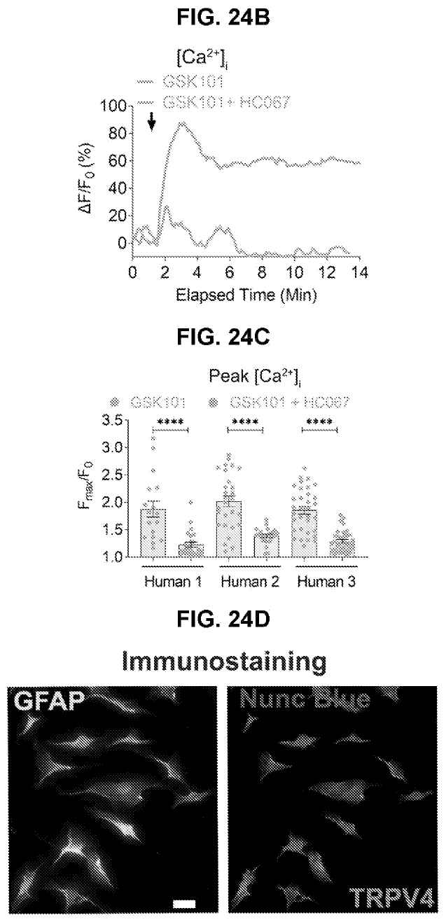

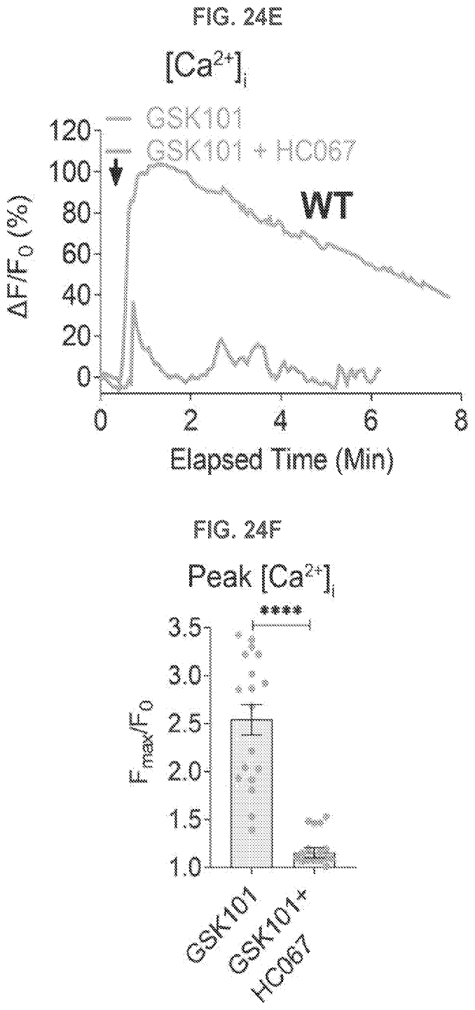

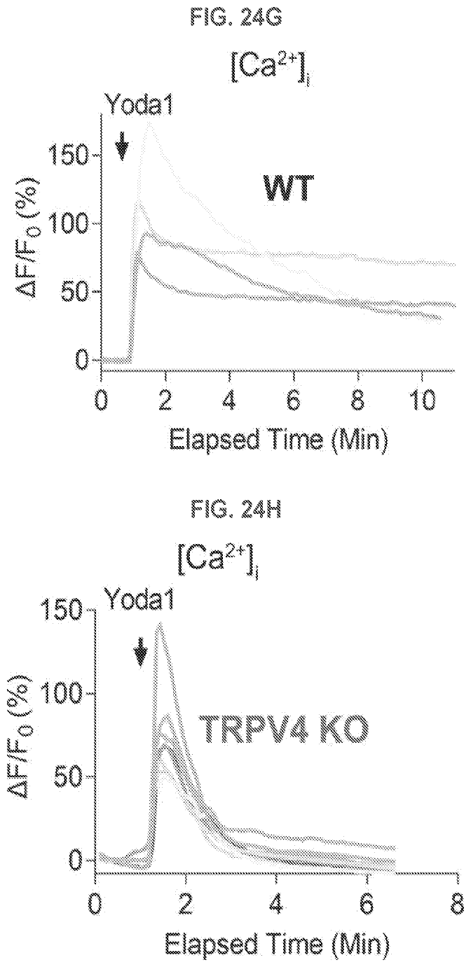

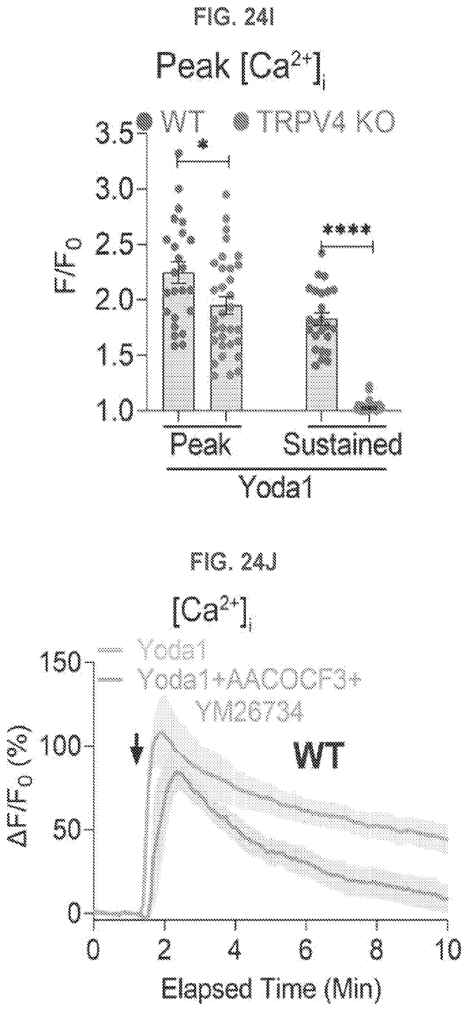

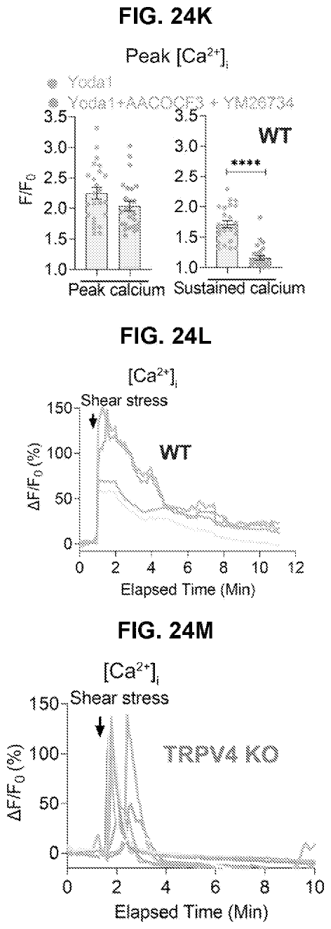

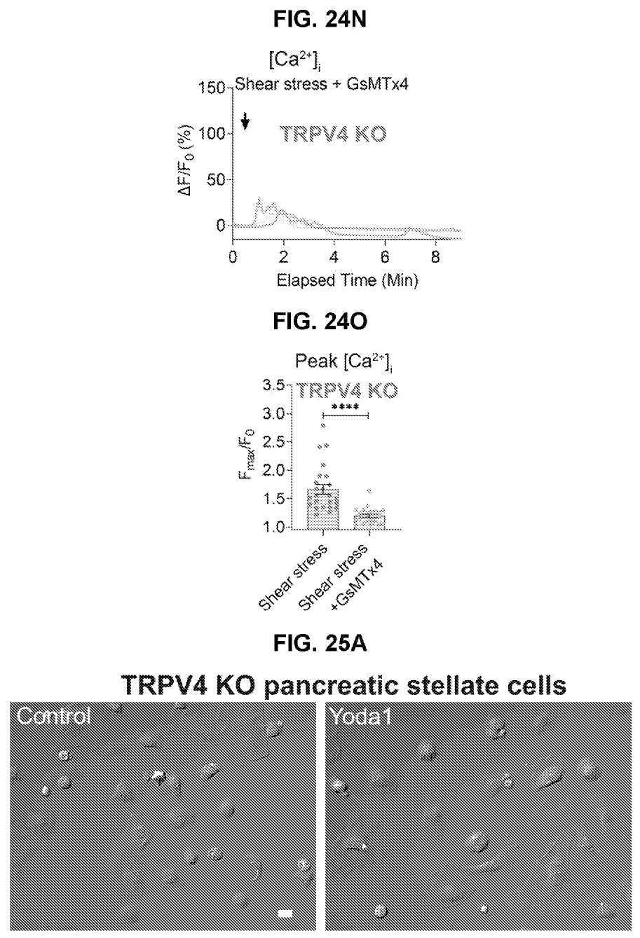

Piezo1 downstream signaling was recently discovered to activate the TRPV4 channel in pancreatic acinar cells and human umbilical vein endothelial cells (HUVECs). Here, functional TRPV4 channels were detected in mouse and human PSCs (FIG. 24A-F). The TRPV4 agonist, GSK1016790A, produced a sustained elevation in [Ca²⁺]ᵢ in quiescent mouse and human PSCs that was blocked with the TRPV4 blocker HC-067047 (FIGS. 24B-C and 24E-F). Although, under certain circumstances, TRPV4-expressing cells respond to mechanical force, this appears to be an indirect effect, since it has not been demonstrated that mechanical manipulations directly cause TRPV4 channel opening. To determine if TRPV4 is involved in Piezo1-mediated changes in [Ca²⁺]ᵢ in PSCs, cells from TRPV4-KO mice were isolated. Yoda1 (25 μM) or prolonged, high shear stress (12 dyne/cm² for 1 minute), which normally produce sustained elevations in [Ca²⁺]ᵢ, caused only transient calcium elevations in TRPV4-null PSCs (FIGS. 24G-I and 24L-M). 5′,6′-epoxyeicosatrienoic acid is an endogenous activator of the TRPV4 channel produced from arachidonic acid via a PLA2-AA cytochrome P450 epoxygenase-dependent pathway. To determine if Piezo1 activates PLA2-AA, the secretory and cytosolic PLA2 inhibitors YM26734 and AACOCF3 were used. Together, YM26734 and AACOCF3 significantly inhibited the Yoda1-mediated sustained elevation in [Ca²⁺]ᵢ in PSCs from WT mice (FIG. 24J-K). Additionally, GsMTx4 blocked the effects of high shear stress (12 dyne/cm² for 1 minute) on the transient [Ca²⁺]ᵢ elevation in TRPV4-null PSCs, confirming that the initial transient calcium influx was due to Piezo1, while subsequent TRPV4 channel opening was responsible for the sustained rise in [Ca²⁺]ᵢ (FIG. 24N-O).

TRPV4-KO Mice are Protected from Pressure-Induced Pancreatic Fibrosis

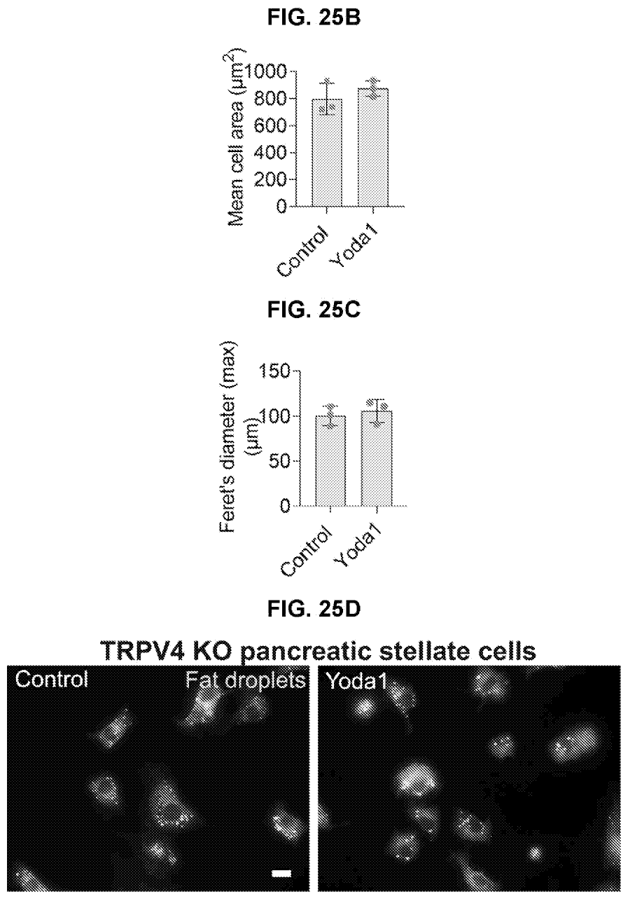

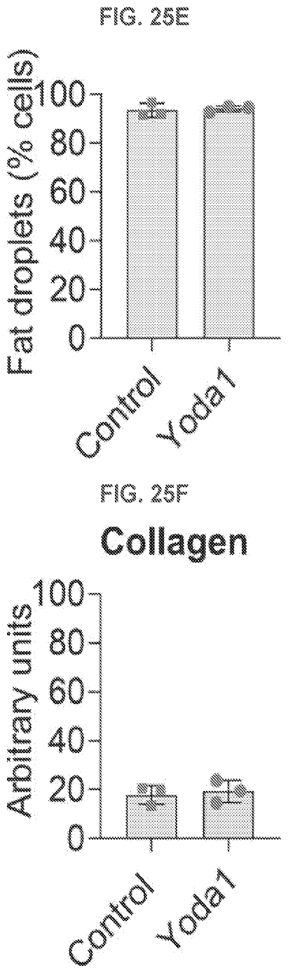

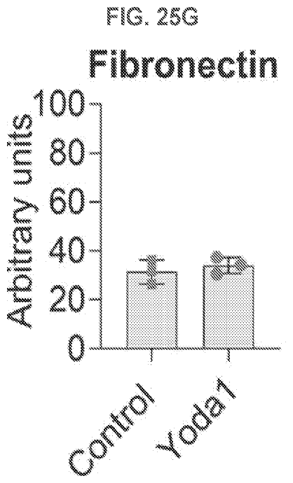

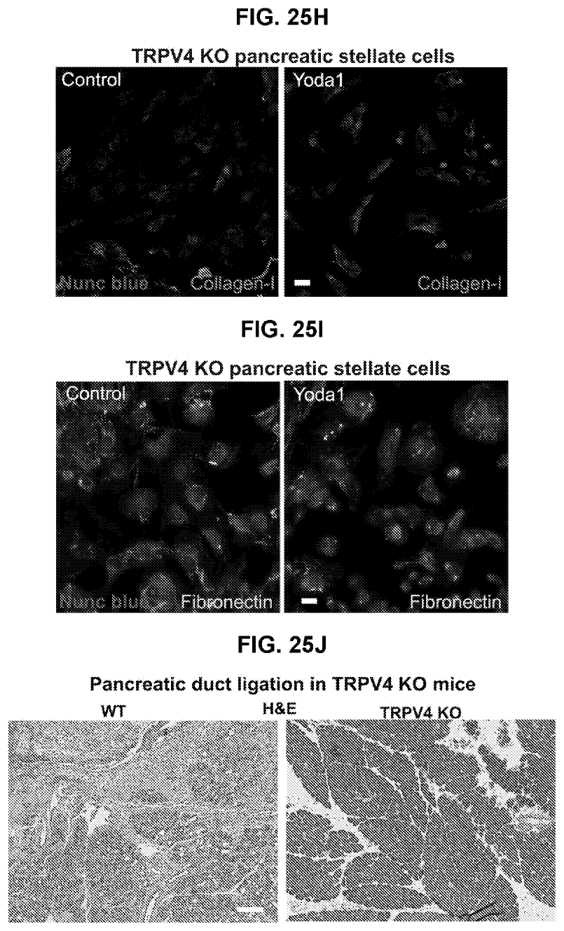

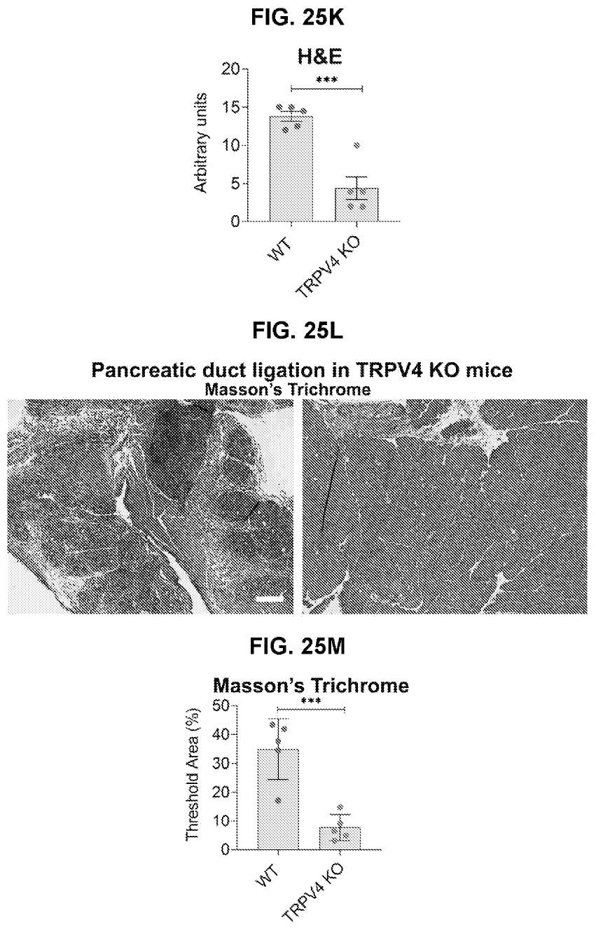

Having determined that the sustained elevation in [Ca²⁺]ᵢ produced by Piezo1 activation requires TRPV4 opening, it was proposed that TRPV4 was responsible for stellate cell activation. To test this possibility, PSCs isolated from TRPV4-KO mice were treated with Yoda1 (25 μM). In contrast to WT PSCs, Yoda1 did not activate PSCs from TRPV4-KO mice, and no changes in PSC activation parameters (cell shape, perinuclear fat droplet abundance, and ECM protein expression) were observed (FIG. 25A-1). If pathological effects of Piezo1 (high-pressure-induced stellate cell activation and fibrosis) require TRPV4 channels, it would be expected that mice lacking TRPV4 would be protected against pressure-induced chronic pancreatitis and fibrosis. As shown in FIG. 25J-M, mice lacking TRPV4 channels were protected from PDL-induced pancreatic fibrosis.

Pancreatic fibrosis is an irreversible complication of chronic pancreatitis that is often accompanied by loss of endocrine and exocrine function. Fibrosis is composed of ECM produced by activated PSCs, which also secrete proinflammatory cytokines that may amplify pancreatic inflammation and accelerate the loss of acinar and islet cells. Pancreatic fibrosis also increases the risk of pancreatic ductal adenocarcinoma (PDAC). The dense desmoplasia found in PDAC is a product of a subtype of activated PSCs known as cancer-associated fibroblasts and poses a major hurdle for chemotherapeutic-based drug delivery. Effective antifibrotic therapies are lacking; therefore, most current efforts are directed at preventing fibrosis by blocking PSC activation. The two most common factors leading to chronic pancreatitis are heavy alcohol use and conditions producing sustained elevations in pancreatic duct pressure, such as duct strictures, cysts, pseudocysts, and obstructive tumors. It is shown here that PSCs exhibit pressure sensitivity by virtue of their expression of the mechanically activated ion channel Piezo1 and that activation of Piezo1 initiates a fibrogenic response. Complete manifestation of the pathological consequences of Piezo1 activation is largely linked to calcium triggered TRPV4 channel opening and its accompanying calcium influx.

The results of this study demonstrate that increased intraductal pressure causes pancreatic fibrosis mediated by PSCs and that PSC sensitivity to pressure is mediated by Piezo1 activation. Brief high shear stress or low shear stress for longer times produced transient increases in [Ca²⁺]ᵢ that were insufficient to activate PSCs. In contrast, Yoda1 or sustained higher shear force produced a sustained elevation in [Ca²⁺]ᵢ and induced PSC activation, manifested by cell elongation, loss of perinuclear fat droplets, and stimulation of profibrotic TGF-β1 and ECM protein (e.g., fibronectin and collagen type I) gene expression. These findings that Piezo1^{GFAP}-KO mice were protected from pressure-induced fibrosis suggest that a Piezo1 blocker could be a possible treatment for pancreatic fibrosis.

Pancreatic fibrosis is an active inflammatory process, accompanied by cell-to-cell contact and dynamic production of inflammatory molecules. Activated PSCs secrete IL-6, IL-1β, monocyte-specific chemokine (MCP-1), and TNF-α; activate tissue-resident macrophages; and recruit inflammatory monocytes, which are major regulators of fibrosis. In human fibrotic tissue and a rat model of chronic pancreatitis, macrophages are in close proximity to PSCs and exacerbate the progression of pancreatic fibrosis through the production of TNF-α and TGF-β1. Importantly, mechanical forces generate proinflammatory responses in macrophages and monocytes in a Piezo1-dependent manner. This is illustrated by the finding that macrophages lacking Piezo1 exhibited decreased inflammation and enhanced wound healing. Piezo1 signaling in myeloid cells also exacerbated a mouse model of pulmonary fibrosis; thus, it appears that Piezo1 can induce fibrosis by acting both directly on stellate cells and indirectly through inflammatory cells. It is conceivable that a blocker of Piezo1 or its downstream signaling pathways could inhibit stellate cell activation and reduce the fibrogenic responses triggered by inflammatory immune cells.

It was recently reported that pancreatic acinar cells express Piezo1 and that elevated pancreatic pressure can cause pancreatitis. During the course of pancreatitis, stellate cells can be activated by proinflammatory molecules, some of which are generated by acinar cells. Thus, it is possible that fibrosis may result from pressure acting directly on PSCs or indirectly on acinar cells through the induction of pancreatitis and subsequent stellate cell activation. In this study, it was observed that PDL produced extensive pancreatic fibrosis in pancreata of WT and Piezo1^{aci}-KO mice, but not Piezo1^{GFAP}-KO mice, indicating that Piezo1 channels in stellate cells rather than acinar cells are responsible for pressure-induced fibrosis. Although acinar cell-mediated inflammatory signaling undoubtedly contributes to the development of chronic pancreatic and fibrosis under certain conditions, it appears that elevated pancreatic pressure may directly promote stellate cell activation and fibrosis.

TGF-β1 is a major profibrogenic cytokine and a target for antifibrotic therapies. Regulation of TGF-β1 function depends on site-specific activation by integrins, and recently, it has been demonstrated that mechanical activation of Piezo1 converts inactive integrins to an active form. In addition to the role of Piezo1 in activation of TGF-β1, these results demonstrate that Piezo1 increases TGF-β1 at the transcriptional level.

Discovery of TRPV4 in PSCs raised the possibility that the pathological effects of Piezo1 on generation of fibrosis may be linked to TRPV4. The results indicated that Piezo1 senses mechanical force and initiates calcium signaling, resulting in TRPV4 activation. In the absence of TRPV4, mechanical force or Yoda1 did not generate the sustained elevation in [Ca²⁺]ᵢ that was necessary to alter PSC morphology, modify perinuclear fat droplet abundance, or initiate fibrogenic responses. Although under certain circumstances, TRPV4 has been variously reported as mechanosensitive, this has not been demonstrated in cell-free systems, and it seems likely that mechanoreceptor properties that were attributed to TRPV4 may be due to true mechanically activated ion channels like Piezo1 that happen to be co-expressed. PSCs appear to be another example of Piezo1 and TRPV4 interdependence.

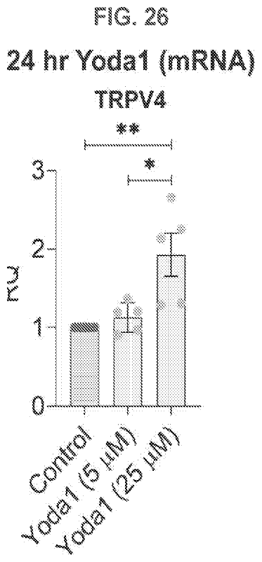

The observation that Yoda1 elevated Piezo1 and TRPV4 mRNA levels in PSCs from WT mice suggests that prolonged pressure may increase the fibrogenic response (FIG. 21K and FIG. 26). It will be interesting to evaluate Piezo1 and TRPV4 in stellate cells of patients with chronic obstructive pancreatitis and to determine if administration of a Piezo1 or TRPV4 blocker can block or reverse obstructive chronic pancreatitis and fibrosis in humans. TRPV4 is expressed in many organs that have mechanosensing properties and exhibit fibrosis in response to injury including the heart, lung, kidney, liver, skin, and intestine. In the liver, TRPV4 expression was increased in hepatic fibrosis and linked to TGF-β1-induced hepatic stellate cell activation. Like hepatic fibrosis, TRPV4 was upregulated in fibrotic pulmonary tissue and TRPV4-KO mice were protected from pulmonary fibrosis. In the heart, TRPV4 converts fibroblasts to a myofibroblast phenotype through a TGF-β1-mediated pathway. Thus, TRPV4 is an established mediator of tissue fibrosis. In addition to the findings in the pancreas, TRPV4 expression was found to be upregulated in a model of alcohol- and high-fat diet-induced pancreatitis, and TRPV4 is expressed in macrophages and linked with inflammation. The observations of this study together with these additional findings support a possible strategy for preventing or treating pancreatic fibrosis by blocking Piezo1 or TRPV4 channels.

### Example 5

### Orthotopic Models of PDAC in WT, Piezo1^{GFAP}KO, and TRPV4-KO Mice

### Mice

Wild type, Piezo1^{GFAP}KO and TRPV4 KO mice have been described in Swain et al., JCI Insight 7: e158288 (2022). Briefly, Piezo1^{fl/fl} mice were a gift from A. Patapoutian (Scripps Research; see Cahalan et al., Elife 4 (2015). To generate Piezo1 deletion in stellate cells, Piezo1^{fl/fl} mice were crossed with B6.Cg-Tg(GFAP-cre/ERT2)505Fmv/J mice (The Jackson Laboratory) to generate the mouse line B6.Cg-Tg(GFAP-cre/ERT2); piezo1^{fl/fl}. To generate conditional genetic Piezo1 deletion in stellate cells, 40 mg of tamoxifen/kg body weight (Millipore Sigma, T5648) was injected i.p. per day for 5 consecutive days. The mice were used 8 days after the last tamoxifen injection. The mouse lines B6.Cg-Tg (GFAP-cre/ERT2); piezo1^{fl/fl} and ptf1a^{CreERTM}; piezo1^{fl/fl} f generated as described in Romac et al., Nat. Commun. 9(1): 1715 (2018) after tamoxifen injection were referred to as Piezo1^{GFAP}-KO and Piezo1^{aci}-KO, respectively. Piezo1^{fl/fl} mice were used as WT in the experiments with Piezo1^{aci}-KO and Piezo1^{GFAP}-KO mice. Seven- to 12-week-old mice (both male and female) were used in the experiments. A small piece of tail of each mouse was used for genotyping. The mouse line with Trpv4 gene deletion (referred to as TRPV4-KO) was obtained from Wolfgang Liedtke (Department of Neurology, Duke University (Kanju et al., Sci. Rep. 6: 26894 (2016) and then bred in-house. C57BL/6J mice (The Jackson Laboratory) were used as WT mice in the experiments with TRPV4-KO mice. Mice were housed under standard 12-hour light/12-hour dark conditions.

### Cells

KPC cells (cancerTools.org) were grown in DMEM/F12, 5% FBS (fetal clone II serum). KPCY cells (Kerafast) were grown in DMEM/F12, 10% FBS and 1× Glutamax.

### Antibodies

GFP: abcam, ab13970
Ki67: abcam, ab16667
YAP: cell signaling technology, S8H1X

### Drugs

Yoda 1 (Tocris, 5586)
GSK2193874 (Sigma, SML0942)

### Methods

### Activation of Human Stellate Cells and Gene Expression Analyses

Mouse and human stellate cells were prepared and maintained in culture as previously described Swain et al., JCI Insight 7: e158288 (2022). Summarized here: Mouse and human PSCs were isolated using collagenase digestion. Modified Krebs Henseleit Buffer (KHB) solution (100 mL) was prepared as described previously in Romac et al., Nat. Commun. 9(1): 1715 (2018). Pancreatic tissue was digested with 2 mg of collagenase NB 8 (SERVA, catalog 17456) dissolved in 10 mL of modified KHB solution containing 1 mg soybean trypsin inhibitor (SBTI 1-S; Millipore Sigma, catalog T9003) and 20 mg BSA (Thermo Fisher Scientific; BP1600-100). Digestion solution (5 mL) was used to inflate the pancreas. Pancreas with 5 mL of digestion solution was incubated in a shaking water bath for 10 minutes at 37° C. The solution was then discarded. The pancreas tissue was cut into small pieces and digested with fresh 5 mL digestion buffer. Tissue was incubated in a shaking water bath for 40 minutes at 37° C. The cells were then separated from tissue by pipetting up-down with a 10 mL pipette and passed sequentially through a 70 μm and a 40 μm cell strainer. The filtrate was centrifuged at 150×g for 3 minutes at room temperature. The cells were washed with 10 mL Leibovitz's media (Thermo Fisher Scientific, catalog 11415-064), passed through a 20 μm filter, and centrifuged at 80 g for 3 minutes at room temperature. The supernate was removed, and isolated stellate cells were plated on a thin-layered Matrigel-coated glass bottom culture plate (MatTek, P35G-0-14-C). Before plating the cells, Matrigel solution (Corning, catalog 354234) at a ratio of 1.5:100 DMEM/F12 (Thermo Fisher Scientific, catalog 11330-032) was poured onto the culture plate and incubated for 2 hours at 37° C. to form a thin layer of Matrigel coating. Matrigel mixture was removed, and the plate was washed with PBS before cells were plated. Cell culture media, DMEM/F12 with 5% FBS was used for mouse stellate cells. Fresh, human pancreatic tissue (provided by Duke University's BioRepository & Precision Pathology Center under IRB approval) was digested with collagenase as described above with modifications. Collagenase (2.5 mg) in 10 mL of modified KHB solution was used for digestion. The digested tissues were filtered through a 100 μm cell strainer. The cells were cultured with DMEM/F12 with 10% FBS in a Matrigel-coated plate. After 24 hours, the cell media was replaced with fresh media to remove unattached and dead cells. After 2 days, cells were immunostained for GFAP and used for experiments. Perinuclear fat droplets were stained with BODIPY 493/503 (4,4-Difluoro-1,3,5,7,8-Pentamethyl-4-Bora-3a,4a-Diaza-s-Indacene; Invitrogen, catalog D3922) to confirm stellate cell quiescence. The cell viability following Yoda1 treatment was analyzed using the Live/Dead cell imaging kit (Thermo Fisher Scientific, catalog R37601) (Swain et al., J. Clin. Invest. 130(5): 2527-2541 (2020). RNAs were isolated using the RiboPure Kit (Invitrogen, catalog AM1924) according to manufacturer's instructions. See Romac et al. Nat Commun.; 9(1): 1715 (2018).

Yoda 1 was resuspended in DMSO at 25 mM stock solution. Yoda 1 was added to stellate cells in culture at a final concentration of 5 or 25 μM and incubated for 24 hours before processing for RNA isolation using the Ribopure kit following manufacturer instructions (Invitrogen AM1924). cDNA was produced using the High Capacity Reverse Transcription kit (appliedbiosystems, 4368814). Realtime PCR was used using TaqMan assays (Life Technologies) and TaqMan Gene Expression Master Mix (appliedbiosystems, 4369016) following manufacturer recommendation.

### Immunostaining of Mouse Stellate Cells

Murine stellate cells from wild type or Piezo1^{GFAP}KO mice were activated with 25 μM Yoda 1 for 4 hours before fixation. Immunostaining staining was performed as described in Swain et al. JCI Insight 7:e158288 (2022). Yap antibody was diluted at 1/100.

### Orthotopic Injections of KPCY Cells

KPCY cells resuspended in PBS+1% matrigel after trypsinization. Either 10,000 or 100,000 cells in 20 μl PBS were injected into the pancreatic tail. Mice (wild type or Piezo1^{GFAP}KO) that had received 10,000 cells were euthanized and organ harvested 28 days after surgery. Mice (wild type or TRPV4-KO) that received 100,000 cancer cells were euthanized at day 20.

### Orthotopic Injection of KPC Cells Followed by Spleen Injection of KPCY Cells

100,000 KPC cells were first injected into the pancreatic tail. 10 days later, 500,000 KPCY cells were injected into the spleen of wild type or TRPV4-KO mice.

Orthotopic injection of KPCY cells with implantation of micro-osmotic pump (Alzet, 1004) filled with either vehicle (6% cavitron W7 HP7 in water—Ashland) or GSK2193874 at 20 mg/mL. Mice were euthanized 20 days later, and organs were measured and harvested.

### Tumor Weight Determination

Solid tumor was dissected from the total pancreas and weighed.

### Preparation of Liver Tissue for RT-PCR

Liver tissue was homogenized using the TissueLyser LT (Qiagen) following the manufacturer's instructions. Isolation of RNA, cDNA synthesis and RT-PCR reactions used the same kit as for stellate cells.

### ELISA IL-6

Blood was collected by cardiac puncture. After 20 minutes on ice, blood was spun in a microfuge for 5 minutes at high speed. Serum was collected and preserved in a −80° C. freezer until use. Serum IL-6 level was measured using the ELISA Kit (R&D; M6000B) following the manufacturer's instruction.

### Liver Tissue Preparation and Immunostaining for Ki67 and GFP Proteins

Liver tissue from wild type or Piezo1^{GFAP}KO mice were frozen in OCT compound (Fisher Healthcare; 4585). Tissue sections were cut at 12 μm thickness. Sections were post-fixed with cold methanol for 20 minutes. KPCY cells express enhanced yellow fluorescent protein (EYFP) that cross reacts with GFP antibody. GFP antibody was used at 1/2000 dilution. Images were taken on a Leica DMi8 microscope.

Liver tissue from wild type or TRPV4-KO mice from the 20 days experiments were first fixed in 4% paraformaldehyde overnight, then replaced subsequently with 10%, 20% and 30% sucrose. Tissues were then embedded in OCT compound and cut at 12 μm. Multiplex immunostainings were performed with Ki67 ( 1/1000) and GFP ( 1/2000) antibodies using a confocal Zeiss 880 microscope.

### Example 6

Mechanical Sensing and Inhibition of Cancer Metastasis with TRPV4 Antagonism

Pancreatic ductal adenocarcinoma (PDAC) is characterized by a dense desmoplasia containing ECM that comprises up to 90% of the tumor volume. This desmoplasia is produced by PSCs that upon stimulation, differentiate into cancer-associated fibroblasts (CAFs), which secrete insoluble ECM and other soluble proteins that stimulate cancer progression. Due to the stiffness of the ECM, mechanical forces are exerted within the microenvironment as cells grow, producing both elevated tissue pressure and shear stress. PSCs are exquisitely sensitive to pressure by virtue of their expression of the mechanically activated ion channel, Piezo1.

Cancer cell migration and invasion are two processes critical to cancer metastasis and both involve transduction of mechanical force. In PDAC, cancer cells migrating to distant tissues encounter a variety of physical restrictions such as extracellular matrix, endothelial barriers, and the inherent solitary morphology of cancer cells which restrict the migration process. To overcome such obstructions, stromal cells surrounding the cancer cells dynamically alter the composition of extracellular matrix via metalloprotease-mediated enzymatic degradation. Additionally, chemo-attractants and growth factors produced from stromal and other cells diffuse from blood vessels modifying the cancer cell's contractile properties for easy migration and invasion. All these events during cancer cell migration to distant tissues are associated with the transduction of mechanical signals into biological activities and are conveyed by mechanical ion channels expressed in stromal cells.

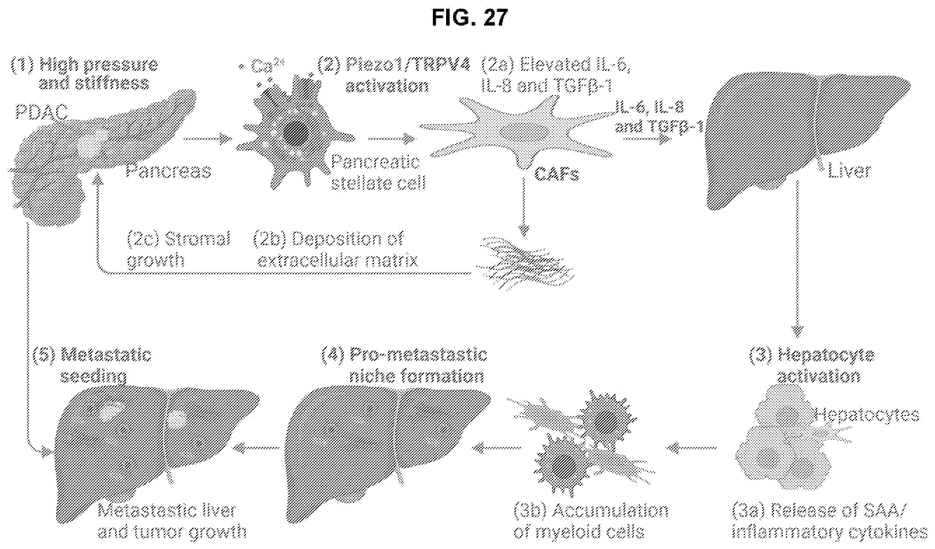

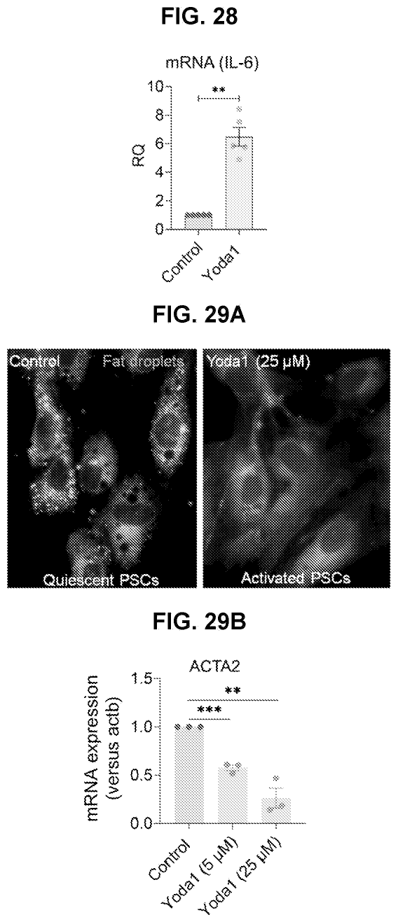

Recently, it has been reported that Piezo1 facilitates breast cancer cell migration and invasion. In glioma, Piezo1 upregulates the expression of genes associated with the tumor microenvironment such as angiogenesis, cell migration, and extracellular matrix deposition. Although Piezo1 has been associated with cancer cell metastasis, its role in PDAC, which harbors a high-pressure environment, is unknown. Metastases are prevalent in PDAC and the liver is the most common site of pancreatic cancer metastases. Although tumor cells in the portal circulation may be trapped in hepatic sinusoids, accumulating evidence indicates that a ‘pro-metastatic niche’ facilitates the spread of cancer cells to the liver. It has been demonstrated that during the early stages of pancreatic tumorigenesis, hepatocytes are primed to attract tumor cells through activation of STAT3 signaling and markers of the niche include increased production and secretion of serum amyloid A1 and A2 (SAA1 and SAA2). Generation of the pro-metastatic niche may require IL-6, which is produced by pancreatic CAFs. Two types of CAFs have been characterized in PDAC, both of which are derived from pancreatic stellate cells. Inflammatory CAFs (iCAFs) express leukemia inhibitory factor (LIF) and IL-6 in contrast to myofibroblast-like CAFs (myCAFs), which produce α-smooth muscle actin (SMA). The inflammatory factors secreted by iCAFs such as LIF, CXCL1, granulocyte colony-stimulating factor (G-CSF), and IL-6 may contribute to tumor progression (FIG. 27).

Piezo1-induced conversion of PSCs to the cancer-associated phenotype is coupled to the ion channel, TRPV4 (FIG. 27). The therapeutic implications of this observation are important because if Piezo1 activation is responsible for the pro-metastatic niche, it may be possible to develop therapeutic strategies to inhibit Piezo1 signaling and reduce pancreatic cancer metastasis. The results of this study show that intrapancreatic injection of KPCY cells in mice with genetic deletion of TRPV4 significantly reduced tumor growth and liver pro-metastatic niche formation factors. Thus, a better understanding of mechanosensing mechanisms underlying PDAC growth and metastasis is essential to improve patient outcomes. It could be possible to prevent PDAC growth and metastases with a TRPV4 antagonist. Notably, TRPV4 antagonists are currently in clinical development. Therefore, these findings may provide a method to reduce PDAC growth and prevent the spread of pancreatic cancer.

### Effects of Pancreatic Pressure on the Conversion of PSCs to a Cancer-Associated Fibroblast Phenotype in Mice

A mouse model of PDL generated by ligating the tail region of the pancreas was used in order to increase pressure within the pancreas and activate PSCs. This model was used in combination with orthotopic transplantation of KPCY (Kras^{LSL-G12D/+}; Trp53^{LSL-R172H/+}; Pdx1-Cre; Rosa26^{YFP/YFP}) cells into the tail region of the pancreas to evaluate the effects of high pressure. In vitro studies were used to model conversion of PSCs into CAFs.

Results have shown that the Piezo1 agonist, Yoda1 (25 μM), caused calcium overload in quiescent pancreatic stellate cells converting them into a fibroblast phenotype characterized by increased expression of fibronectin and IL-6 (FIGS. 18I, 21F-G, and 28). Yoda1 induced a sustained calcium rise that was not observed in PSCs from mice with genetic deletion of Piezo1 in stellate cells (Piezo1^{GFAP}-KO) (FIG. 18C). Moreover, Piezo1^{GFAP}-KO mice were protected from pressure-mediated PDL-induced fibrosis (FIG. 15J-K). These data suggest that activation of Piezo1 converts PSCs into an iCAF phenotype and are consistent with the hypothesis that PSCs produce soluble IL-6 and other factors which induce a pro-metastatic niche within the liver that enhance pancreatic cancer metastases.

To test this hypothesis, the effects of pancreatic pressure on the conversion of PSCs to an iCAF phenotype and their ability to produce factors that contribute to the pro-metastatic niche are determined. KPCY cells (100,000 cells in 25 μL PBS) were injected into the pancreatic tail region with and without pancreatic duct ligation, which produces a high-pressure environment (FIG. 15B). Pancreatic pressure, measured with a pressure transducer (APT300 Hg, Harvard Apparatus), and intratumoral stiffness, measured by atomic force microscopy, are evaluated over time (e.g., days 2 and 5). Both wild-type and Piezo1^{GFAP}-KO mice are used for all assays. Experiments in wild-type mice will demonstrate the ability of pancreatic pressure to modify PSC phenotype and studies in Peizo1^{GFAP}-KO mice will determine if Piezo1 in PSCs is responsible for these changes.

To evaluate the iCAF phenotype, fibronectin surrounding cancer cells and mRNA and serum levels of LIF, IL-6, IL-1, IL-8, CXCL1, and G-CSF are measured. It will also be determined if cancer cell transplantation changes the local pressure surrounding the tumor and if this correlates with PSC activation and iCAF conversion. This study will determine how high pancreatic pressure during PDAC causes Piezo1 activation that leads to the conversion of PSCs to iCAFs and production of factors that contribute to the pro-metastatic niche.

### Effect of Piezo1 on Generation of Pro-Metastatic Factors in PSCs

The effects of Piezo1 on stellate cells in vitro are examined by treating quiescent human and mouse pancreatic stellate cells with the Piezo1 agonist, Yoda1, and measuring mRNA levels of pro-metastatic factors such as IL-6, IL-8, LIF, G-CSF, IL-11, IL-1, and MMPs. Quiescent primary mouse and human stellate cells are used to investigate Piezo1's conversion of PSCs to CAFs. PSCs are isolated using collagenase digestion. To maintain quiescence, isolated stellate cells are plated on a thin-layered Matrigel-coated glass bottom culture plate (MatTek, P35G-0-14-C). After 24 hr, the cell media are replaced with fresh media to remove unattached and dead cells. After two days, a few samples of cells are immunostained for glial fibrillary acidic protein (GFAP) and perinuclear fat droplets are stained with BODIPY 493/503 (4,4-Difluoro-1,3,5,7,8-Pentamethyl-4-Bora-3a,4a-Diaza-s-Indacene; Invitrogen, catalog D3922) to confirm stellate cell quiescence and remaining cells are used for experimentation (FIG. 29A).

It has been demonstrated that the Piezo1 agonist, Yoda1 (25 μM), activates human and mouse stellate cells, and activated stellate cells produce elevated IL-6 and increased expression of fibronectin, which are characteristic of iCAFs in PDAC. Notably, iCAFs produce inflammatory factors such as CXCL1 and granulocyte colony-stimulating factor (G-CSF), which contribute to tumor progression. To further confirm that Piezo1 converts stellate cells into an iCAF phenotype, human and mouse quiescent stellate cells are treated with Yoda1 and the mRNA levels of inflammatory factors such as LIF, IL-6, IL-1, IL-8, CXCL1, and G-CSF are measured. RNAs are isolated using the RiboPure Kit (Invitrogen, catalog #AM1924) according to the manufacturer's instructions. In vitro experiments with Yoda1 will mimic pressure-induced Piezo1 activation in PSCs during pancreatic tumor growth.

### Effect of Piezo1 on CAF Differentiation

In breast cancer, two cell surface molecules, CD10 and GPR77, define a specific CAF subset that sustains cancer stemness and chemo-resistance and promotes tumor formation. Although CAFs from chemo-resistant and chemo-sensitive breast cancer patients have different mRNA signatures, these properties were not distinguishable by conventional fibroblast markers, such as FSP1, α-SMA, PDGFRβ, FAP, and collagen-I. Similar to breast cancer, high pressure and stiffness are two key mechanical factors in PDAC. It is possible that CAFs in PDAC also have distinctive features that convey cancer stemness and chemo-resistance. As an initial step in exploring this possibility, it was determined if CD10 and GPR77 are expressed in mouse and human PDAC tissue by comparing mRNA levels and immunostaining in wild-type mouse PDAC and Piezo1^{GFAP}-KO PDAC mice. The effects of Piezo1 on CD10 and GPR77 mRNA levels are determined in Yoda1-treated human and mouse stellate cells. Preliminary results have demonstrated that Yoda1 treatment decreases ACTA2 and increases CD10 expression in human stellate cells, suggesting that Piezo1 channel activation induces an iCAF phenotype (FIG. 29B). Further experiments will determine if Piezo1 induces heterogeneity in CAFs.

### Effects of Piezo1 on PSC-Mediated Cancer Cell Migration and Invasion In Vitro

To determine whether inflammatory factors secreted from Yoda1-treated stellate cells trigger cancer cell migration and invasion, transwell cell migration and invasion assays are used, respectively. Initially, human stellate cells are treated with and without Yoda1 for 24 hr and after treatment, the cell culture media are passed through a 10 kDa filter to remove Yoda1 from samples. Human pancreatic cancer cell migration and invasion assays are performed in the collected stellate cell cultured media to determine if inflammatory factors produced by activated stellate cells can trigger cancer cell migration and invasion. The effects of Yoda1 on the induction of inflammatory factors (e.g., cytokines, chemokines, and acute phase proteins) are initially screened using the human cytokine array available from the R&D system (Catalog #ARY005B). The effects of individual factors which are significantly elevated are further analyzed by inhibiting their effects with specific inhibitors or using an antibody immunoneutralization approach.

### Characterization of the Signaling Pathway Downstream of Piezo1 Activation in PSCs

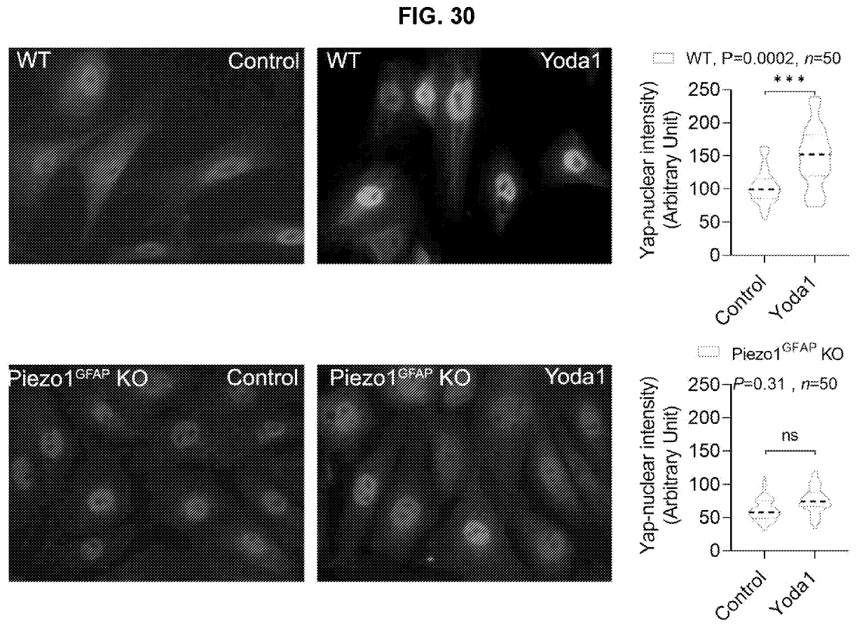

The Hippo pathway target, Yes-associated protein (YAP), is associated with metastasis in breast cancer by elevating TEAD transcriptional activity. YAP and TAZ are homologous mechanoregulatory profibrotic transcription cofactors that regulate cell proliferation, migration, and invasion, and YAP/TAZ is known to regulate fibroblast activation. In PDAC, the mechanosensing role of YAP/TAZ is unknown. Preliminary data show that the Piezo1 agonist Yoda1 increases YAP nuclear localization (FIG. 30). No changes were observed in stellate cells from Piezo1^{GFAP}-KO mice, suggesting that Piezo1 signaling may operate through the YAP/TAZ pathway, converting quiescent stellate cells into CAFs. The Piezo1 agonist Yoda1 is used in human and mouse wild-type and Piezo1^{GFAP}-KO stellate cells. YAP/TAZ nuclear localization is quantified, and the activity of YAP and TAZ (phosphorylated state) is analyzed by western blotting. A YAP blocker will be used to modulate YAP/TAZ activity by transfecting plasmid containing gene sequence that regulates YAP/TAZ activity. RNA sequencing of Yoda1-treated and non-treated human stellate cells should indicate the Piezo1-mediated molecular pathways.

### Effects of Piezo1 on Liver Pro-Metastatic Niche Formation and Pancreatic Cancer Metastases

To determine if Piezo1 participates in pancreatic cancer cell metastases to the liver, orthotropic injection of KPCY cells (100,000 cells in 25 μL PBS) is used. The effects of pancreatic pressure on PSC activation are determined by performing pancreatic duct ligation in mice compared to mice with normal pancreatic pressure (sham surgery, no duct ligation). It is proposed that pancreatic pressure alone is sufficient to convert PSCs into iCAFs that produce circulating factors that induce the pro-metastatic niche. Therefore, to determine the effects of pancreatic pressure on tumor cell seeding in the liver in the absence of a primary pancreatic tumor, KPCY cells are injected into the spleen of mice, which is an established method for evaluating cancer cell seeding from the blood, with and without pancreatic duct ligation (to produce elevated pancreatic pressure and PSC activation). KPCY cells are injected into the spleen 10 days after PDL, and the liver tissue is analyzed 10 days after injection. By measuring blood levels of IL-6 and other factors, the proposed pro-metastatic soluble factors secreted from iCAFs that are essential in the development of the liver pro-metastatic niche are identified. In an alternate approach, KPC cells will be transplanted into the tail region of the pancreas. After ten days, KPCY cells, which express yellow fluorescent protein, are injected into the spleen and cells spreading to liver can be easily visualized by their yellow fluorescence (FIG. 32). This experiment should reveal whether orthotopically transplanted KPC cells generate pro-metastatic niche factors that can affect the spread of KPCY cells from the spleen to the liver.

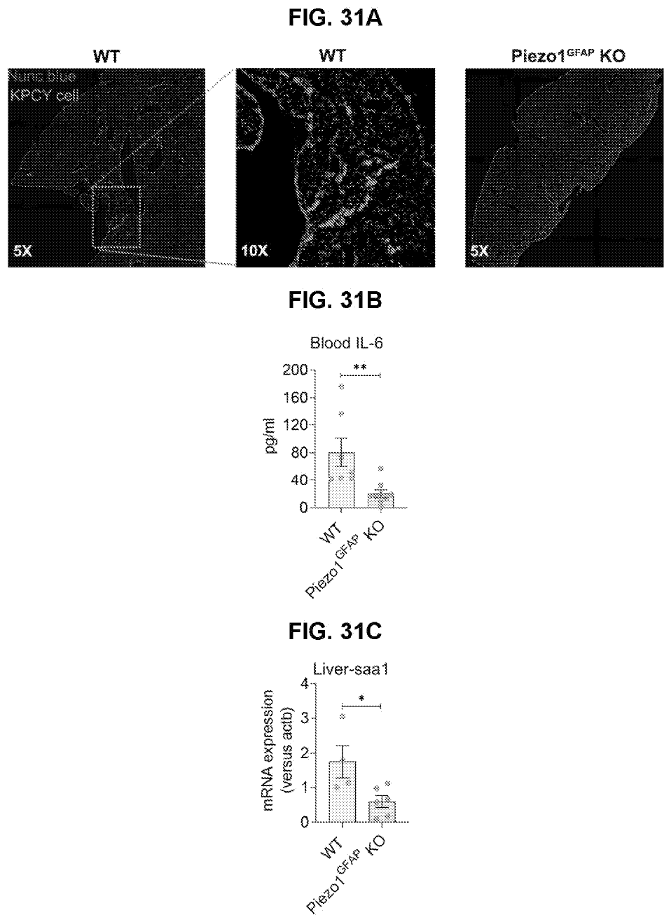

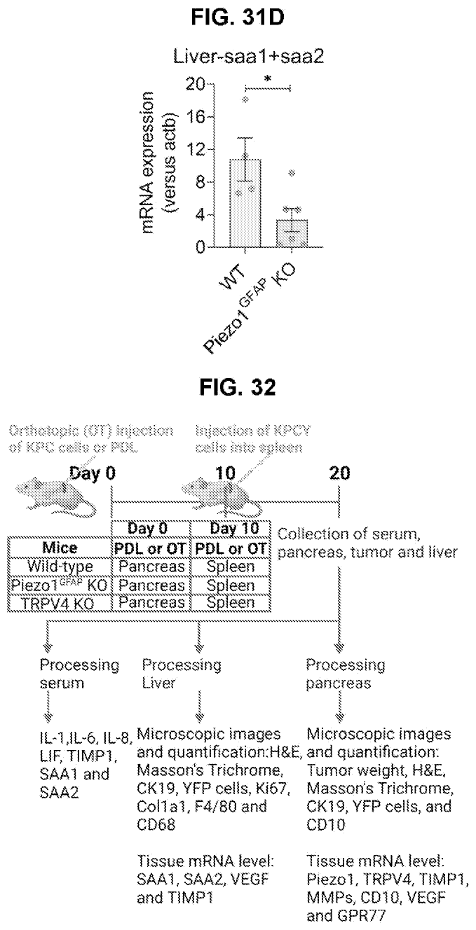

In preliminary observations following KPCY orthotopic transplantation, it was observed that blood IL-6, mRNA levels of the hepatocyte-derived factors SAA1 and SAA2, and the number of metastatic pancreatic cancer cells were reduced in Piezo1^{GFAP}-KO mice (FIG. 31), indicating that Piezo1 in stellate cells is important for hepatic metastasis.

It was recently found that the TRPV4 channel, which is activated downstream of Piezo1, is important for stellate cell activation. Therefore, both Piezo1^{GFAP}-KO and TRPV4 KO mice are tested in these studies, where it is expected that Piezo1 deletion in the stellate cells or global TRPV4 deletion will prevent pancreatic cancer cell metastasis. To determine whether Piezo1 activation is responsible for liver pro-metastatic niche creation and pancreatic cancer metastases, a model of orthotopic transplantation of KPC cells in wild-type, Piezo1^{GFAP}-KO, and TRPV4 KO mice are used (FIG. 32). Increased intrapancreatic pressure is generated by ligating the distal portion of the pancreatic duct (PDL). The effects of increased pressure on pancreatic tumor growth are evaluated by injecting KPC cells into the distal pancreas. Although transplanted KPC cells may metastasize, the rate of early metastasis is low and a more reliable method for assessing metastatic factors is achieved by injecting fluorescently labeled KPCY cells into the spleen. Detection of fluorescent KCPY cells in the liver is indicative of the efficiency of the spread of KPCY cells and would be an indicator of a pro-metastatic condition. This approach should allow for the assessment of the effects of PDL on metastasis independent of orthotopically transplanted KPC cells. Together, these studies should allow for the determination if high intrapancreatic pressure can trigger the formation of the pro-metastasis niche in the liver mediated by Piezo1 activation in pancreatic stellate cells.

mRNA levels of IL-6, IL-8, vascular endothelial growth factor (VEGF), MMPs, and TIMP1 are measured from pancreas tissues, and the hepatocyte-derived factors SAA1, SAA2, and hepatocyte growth factor-like protein (HGFL) are measured in liver tissue from wildtype, Piezo1^{GFAP}-KO, and TRPV4 KO mice. Male and female mice are analyzed separately and combined. Pro-metastatic factors are quantified in serum and liver and pancreas tissue will be processed for microscopic and transcriptional analysis. An important mechanism by which IL-6 is believed to induce the liver pro-metastatic niche is through the activation of STAT3 in hepatocytes. Therefore, P-STAT3 levels will also be quantified in hepatic tissues by western blot. Quantitative analysis of KPCY cells and proliferation of tumors in the liver are determined by immunostaining using EYFP and Ki-67 antibodies, respectively. Masson's trichrome staining will be used to quantify collagen deposition surrounding tumors in the liver. Macrophage recruitment surrounding the tumor is assayed using CD68, F4/80 antibodies. This study should establish whether intrapancreatic pancreatic pressure or stiffness contributes to the liver pro-metastatic niche and pancreatic cancer metastases through Piezo1 or TRPV4 activation in stellate cells.

### Effects of TRPV4 Inhibition on Pancreatic Cancer Growth, Metastasis, and Mouse Survival in an Orthotopic Transplantation Model of PDAC

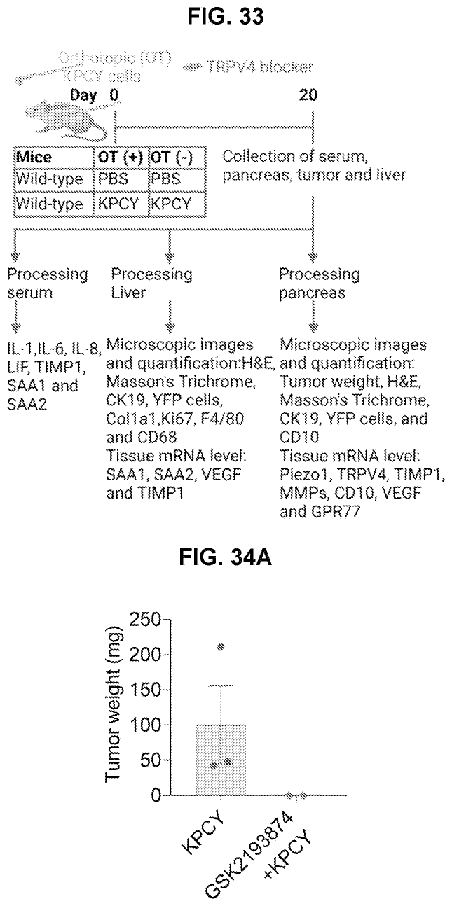

This study determines if a pharmacological inhibitor of Piezo1 signaling can be used to treat pancreatic cancer. Using the experimental approaches as described herein, it will be determined whether TRPV4 mediates pressure-activated PSC activation and generation of pro-metastatic factors IL-6, LIF, G-CSF, IL-11, IL-1, and MMPs. Experiments are performed in wild-type mice with orthotopic KPCY cell transplantation and orthotopically transplanted mice treated with the TRPV4 antagonists, GSK2798745 or GSK2193874 (FIG. 33).

The effects of pharmacological TRPV4 inhibition are compared to orthotopic transplantation studies conducted in TRPV4 KO mice. Notably, transplanted cancer cells are not deficient in TRPV4, and therefore, the effects of pharmacological TRPV4 blockade may have additional effects to those observed in TRPV4 KO mice. Cancer metastases to the liver are assessed by measuring liver volume, weight, and histology. Body weight and survival of mice will also be monitored. This study should demonstrate whether TRPV4 activation contributes to the pro-metastatic niche and is responsible for pancreatic cancer metastases. Preliminary data are consistent with the hypothesis that TRPV4 gene deletion prevents the activation of PSCs, conversion of PSCs to a fibroblast phenotype, and pressure-induced pancreatic fibrosis. These results are important because it may be possible to prevent PDAC metastases with a TRPV4 antagonist, which are currently in clinical development. Therefore, these findings could provide a method to prevent conversion of PSCs to CAFs, generation of pro-metastatic niche promoting factors, and ultimately liver pro-metastatic niche formation. To determine if this is a possible strategy, the effects of the KPC model in TRPV4 KO mice are evaluated.

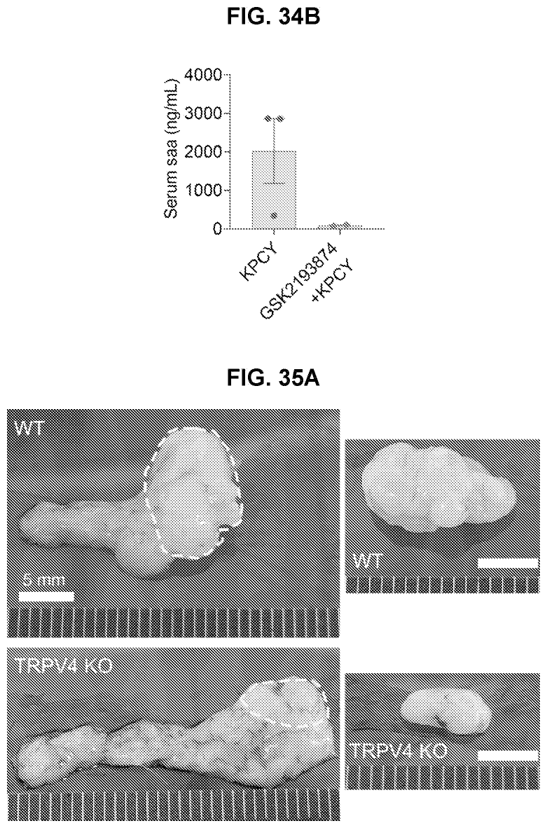

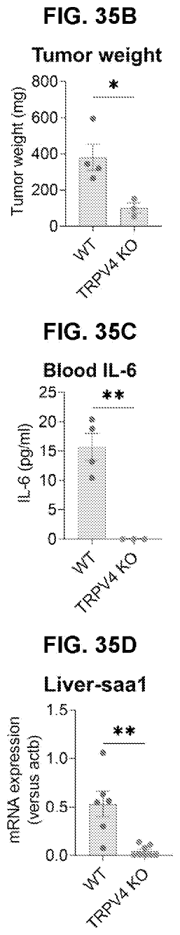

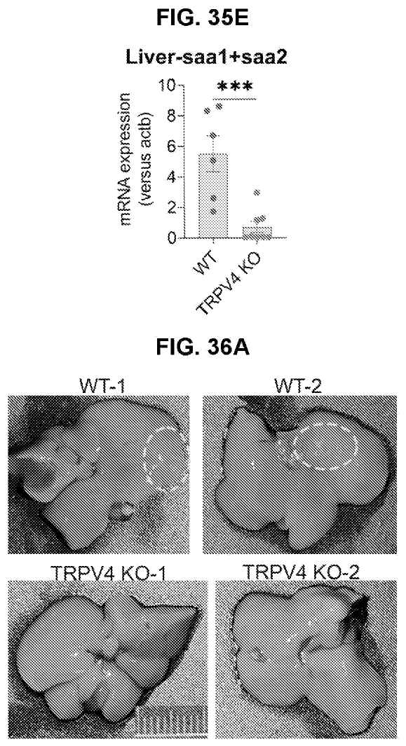

Preliminary data using this model found that the TRPV4 blocker, GSK2193874, inhibited pancreatic cancer growth and reduced serum SAA levels (FIG. 34A-B). Different doses of the TRPV4 blocker GSK2193874 are tested, and results are confirmed by using a second TRPV4 blocker, GSK2798745, to determine whether TRPV4 could be a drug target in PDAC. In preliminary experiments, it was observed that genetic deletion of TRPV4 (TRPV4 KO mice) reduces pancreatic tumor growth and pro-metastatic factors such as IL-6 and serum SAA1 and SAA2 in an orthotopic PDAC mouse model (FIG. 35A-E).

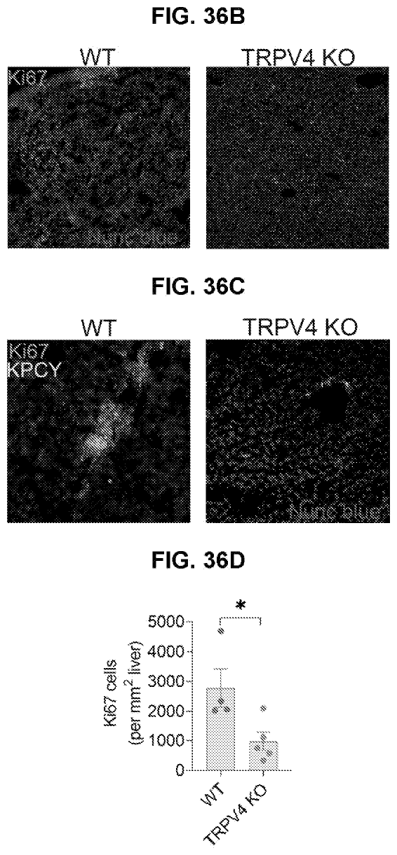

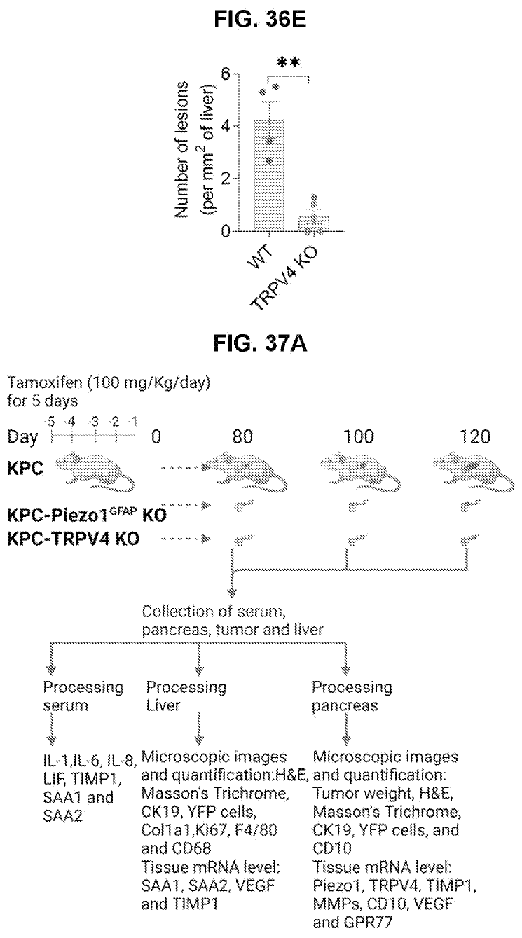

To determine if reduced pro-metastatic factors in TRPV4 KO mice attenuate metastatic liver colonization in PDAC, preliminary studies injected KPC cells into the tail region of the mouse pancreas in order to initiate PDAC. After ten days, PDAC mice were intrasplenically injected with KPCY cells, and the liver was analyzed ten days later to determine if KPCY cells had spread to the liver. It was found that the pancreatic tumor was smaller and metastatic colonization was less in TRPV4 KO mice compared to WT mice (FIG. 36A-E).

This study should determine if the TRPV4 channel operating downstream of Piezo1 signaling drives the high pancreatic pressure regulated liver pro-metastatic niche and pancreatic cancer metastases. These findings should provide a targetable approach to treat PDAC growth and reduce metastases to the liver.

### Effects of Stellate Cell Specific Piezo1 and TRPV4 Gene Deletion on Pancreatic Cancer Growth, Metastasis, and Mouse Survival in a Genetically Modified Mouse Model of PDAC

Kras gene mutations play a crucial role in PDAC initiation and progression and are observed in more than 90% of PDAC patients. In addition, tumor suppressor gene T53 mutations are frequently seen in PDAC patients, and favor PDAC progression. Although several signaling factors are required for Kras-induced pancreatic tumor development, the JAK/STAT3 signaling pathway is considered important. Typically, PDAC begins from a microscopic noninvasive precursor lesion and progresses through several higher-grade lesions in stages of pancreatic intraepithelial neoplasia (PanIN) development. Desmoplastic features accompanying PanIN lesions are mainly created by activated PSCs (i.e., CAFs).

In this study, it is hypothesized that pancreatic CAFs secrete IL-6, which can activate STAT3 signaling and increase the production of serum amyloid A1 and A2 (SAA1 and SAA2) in hepatocytes, the markers of the metastatic niche. To test this hypothesis, the KPC mouse model of PDAC is used with and without genetic deletion of Piezo1 in stellate cells (Piezo1^{GFAP}-KO) or TRPV4 KO to determine if Piezo1 or TRPV4 channels are required for desmoplasia to form and for cancer metastases to develop. KPC mice display disease progression and metastases that resemble human PDAC. KPC mice [Kras^{LSL-G12D};p53^{LoXP}; Pdx1-CreER triple mutant model of tamoxifen-inducible PDAC (Jackson Laboratory)] are used and are crossed with B6.Cg-Tg (GFAP-cre/ERT2); piezo1^{fl/fl} (Piezo1^{GFAP}-KO) mice to generate Kras^{LSL-G12D}; p53^{LoXP}; Pdx1-CreER; Piezo1GFAP-KO; (named KPC-Piezo1GFAP-KO) and KrasLSL-G12D; p53LoxP; Pdx1-CreER mice are crossed with TRPV4 KO mice to generate Kras^{LSL-G12D}; p53^{LoxP}; Pdx1-CreER; TRPV4 KO mice (named KPC-TRPV4 KO). In general, KPC mice develop precursor lesions or early PanIN within 8-10 weeks after tamoxifen administration. At this early stage, an inflammatory response is observed by infiltration of F4/80 positive cells (macrophage marker) and a strong inflammatory response continues as PanIN lesions grow and cancer metastasizes. However, the precise role of pancreatic stromal cells, particularly stellate cells in the inflammatory response, and recruitment of myeloid cells during cancer cell metastasis is unclear. By 16 weeks after tamoxifen, most KPC mice develop invasive PDAC, biliary obstruction, and weight loss.

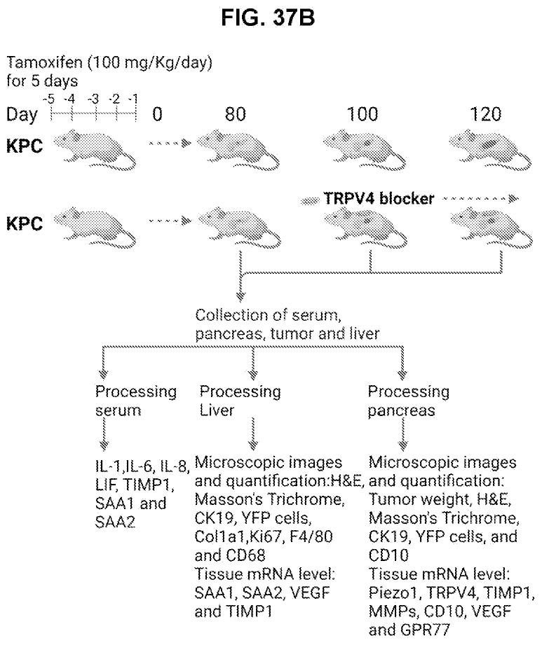

To determine if KPC-Piezo1^{GFAP}-KO and KPC-TRPV4 KO mice exhibit reduced tumor growth and metastasis, an experimental approach was utilized as illustrated in FIG. 37A-B. Body weight, pancreatic tumor growth, pro-metastatic factors, and liver metastases (liver volume, weight, and histology) are measured in KPC, KPC-Piezo1^{GFAP}-KO, and KPC-TRPV4 KO mice on days 80, 100, and 120 after tamoxifen and, as appropriate, the study is extended to assess survival. Pancreatic precursor lesions are evaluated by anti-CK19 and histological assessment of early and late PanIN.

### Effects of TRPV4 Inhibition on Pancreatic Cancer Growth and Metastasis and Mouse Survival in a Genetically Modified Mouse Model of PDAC

To determine if a TRPV4 antagonist can be used to treat pancreatic cancer and prevent pancreatic cancer metastases, KPC mice are treated with GSK2193874 or GSK2798745. Drug are administered by an osmotic mini-pump implanted at day 100 after tamoxifen treatment. The results of this study should reveal if a TRPV4 blocker can attenuate further pancreatic tumor growth and metastasis. These findings should indicate if TRPV4 blockade can be used to treat PDAC.

### Example 7

### Method for Treatment and Prophylaxis of Keloids

Keloid is overgrowth of granulation tissue at the site of a scar beyond the normal boundaries of healing and is composed primarily of collagen. Type Ill collagen predominates in early stages and type I collagen appears in later stages of keloid growth. Keloids are generally firm or rubbery lesions that often grow in a claw-like pattern. Although they appear as tumors, keloids are benign, non-malignant tissue and are non-contagious. Keloids are more common in individuals of African, Asian, Hispanic, and European descent. In the United States keloids are 15 times more frequent in people of sub-Saharan African descent than in people of European descent. There appears to be a genetic predisposition as keloids develop more commonly in those individuals with a family history of keloids. Keloids also develop more commonly in individuals between the ages of 10 and 30 years.

Keloids usually develop at the site of skin injury or trauma such as surgery, skin piercings, scratches, burns, chickenpox scars, vaccination sites, or acne. As fibrotic tumors, keloids contain atypical fibroblasts and extracellular matrix that is composed of collagen, fibronectin, elastin, and proteoglycans. They are relatively acellular although fibroblasts can be seen throughout the lesions. Keloids may cause physical disfigurement, pain, and itching.

Treatments for keloids or strategies to prevent keloids are often ineffective but may include intra-lesional corticosteroids (e.g., triamcinolone acetonide), cryosurgery, radiation, laser therapy, pressure therapy, silicone gel sheeting, interferon, 5-fluorouracil, and surgical excision, as well as various topical agents including flavonoids and imiquimod creams. See Gauglitz et al., Mol. Med. 17: 113-125 (2011).

Keloids are characterized by a dense deposition of collagen and other extracellular matrix (ECM) that comprise most of the tumor volume. This matrix is produced by fibroblasts that upon stimulation, differentiate into myofibroblasts which secrete insoluble ECM and other soluble proteins that may contribute to keloid progression. Due to the stiffness of the ECM, mechanical forces are exerted within the microenvironment as cells grow, producing both elevated tissue pressure and shear stress. In addition, stretching or pressure on the skin may exert mechanical forces on keloids. Fibroblasts are exquisitely sensitive to pressure by virtue of their expression of the mechanically activated ion channel, Piezo1. Stimulation of Piezo1 by mechanical force or shear stress opens the channel and allows extracellular calcium to flow into the cell. Two chemical tools have been used to study Piezo1 activation. Yoda1 is a highly selective chemical agonist for Piezo1 and GsMTx4 (a tarantula toxin) inhibits Piezo1. Piezo1 is expressed in several tissues including endothelium, urinary bladder, kidney, lung, pancreas, the gastrointestinal tract, and skin. Piezo1 plays a physiological role in vascular and lymphatic development, and acts as a homeostatic sensor to control epithelial cell division. Piezo1 also affects stem cell fate decisions, cell migration, red cell volume and cell osmotic pressure. Apart from its physiological functions, Piezo1 is involved in high shear stress-mediated endothelial dysfunction, and pancreatitis. Mechanical stress activates Piezo1 and collagen synthesis in pancreatic stellate cells, a necessary stromal cell type responsible for ECM deposition in the pancreas. In mice, genetic deletion of Piezo1 in stellate cells prevented stellate cell activation and reduced ECM protein synthesis.

The sustained actions of Piezo1 on intracellular calcium are mediated by a second ion channel—transient receptor potential vanilloid subfamily 4 (TRPV4). TRPV4 is a non-selective cation channel permeable to calcium ions. Physical force (e.g., shear stress or membrane stretching) and hypotonic cell swelling activate TRPV4, but this sensing appears to be indirect. Piezo1 induces the activation of phospholipase A2, which causes TRPV4 channel opening. Activation of phospholipase A2 activity triggers the release of arachidonic acid and its metabolite, 5′,6′-epoxyeicosatrienoic acid (5′,6′-EET) which is an endogenous ligand for TRPV4. TRPV4 is highly expressed in both human and mouse fibroblasts, and recent data showed that the pattern of Piezo1-induced sustained elevation in intracellular calcium cells was reproduced by selective chemical activation of Piezo1 or TRPV4 in vitro. Collagen-producing activation was accompanied by increased expression and deposition of fibrogenic proteins. Thus, sustained elevation in [Ca²⁺]ᵢ produced by Piezo1 activation requires TRPV4 opening. TRPV4 was proposed as responsible for fibroblast activation resulting in collagen production. Consistent with this, mice lacking TRPV4 are protected against pressure-induced fibrosis.

Injury to skin initiates a fibrotic response through the stimulation of myofibroblasts which secrete collagen. Myofibroblasts, characterized by expression of glial fibrillary acidic protein (GFAP) and α-smooth muscle actin expression, reside in close proximity to keratinocytes and other dermal elements in the skin. GFAP-expressing cells are present in both the epidermis and dermis. However, epidermal cells express a higher level of GFAP than dermal cells. Piezo1 and GFAP co-localized in the majority of cells. Collagen-producing cells in the pancreas (known as stellate cells) express GFAP and Piezo1 and respond to mechanical force. Prolonged stimulation induces these cells to produce collagen. High levels of Piezo1 and TRPV4 are expressed in human keloid scar and based on the discovery that Piezo1 mediated mechano-signaling pathways induced TRPV4 channel activation is required for abnormal collagen synthesis in pancreas, suggests that this phenomenon may operate in keloid where it would be responsible for high levels of collagen deposition and unusual skin growth.

Human keloid, 5-μm thick paraffin-imbedded formalin-fixed tissues were used for immunohistochemistry and immunostaining. For immunostaining, tissues were deparaffinized following the Abcam deparaffinization protocol, and treated for antigen retrieval using the Abcam antigen retrieval buffer, catalog no; ab93684, as per manufacturer's instructions. The fixed tissues were treated with 0.15% Triton X-100 for 15 min. and then blocked with 2% bovine serum albumin for 1 hr at room temperature. Human keloid tissues were immunostained with rabbit anti-TRPV4 antiserum (Alomone, ACC-034, 1:250), rabbit anti-Piezo1 antiserum (Proteintech, 15939-1-AP, 1:300), rabbit anti-collagen type I antibody (Abcam, ab34710, 1:1000) overnight at 2-8° C. Secondary goat anti-rabbit IgG Alexa Flour Cy3, or secondary goat anti-chicken IgG Alexa Flour 488 (Jackson ImmunoResearch), was used for 45 min. at room temperature. Nuclei were stained with Nunc blue (Invitrogen, R37606), and tissues were mounted with ProLong Gold antifade reagent (Invitrogen). All stained images were taken with a Zeiss 880 airyscan fast inverted confocal microscope.

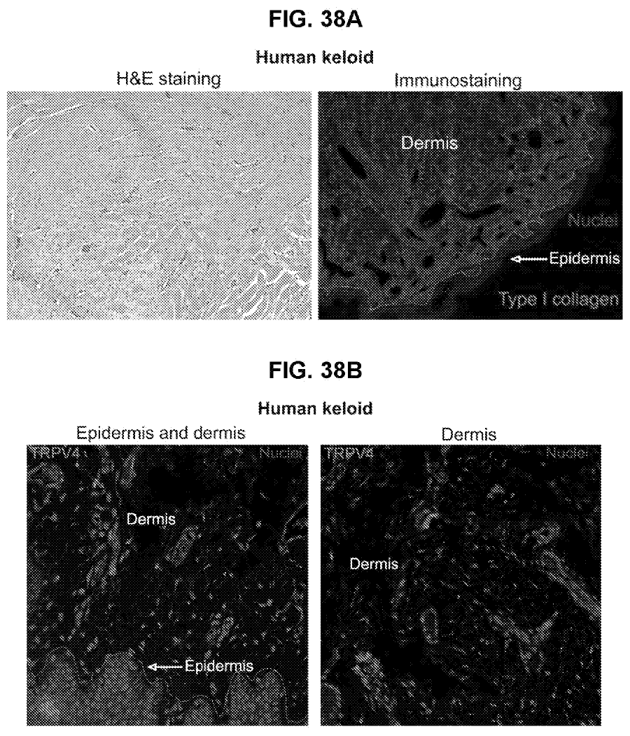

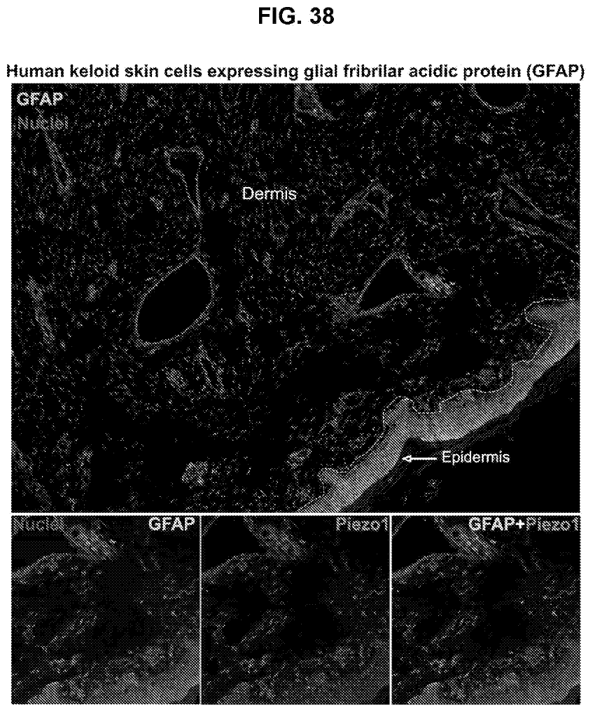

To determine if Piezo1 and TRPV4 are involved in collagen deposition in keloid formation, human keloid was examined for the expression of Piezo1 and TRPV4. As shown in the FIG. 38A-B, and FIG. 39, human keloids contain abundant collagen with cells scattered throughout the thick fibrous strands. TRPV4 immunostaining is detectable in cells of the dermis and in cells scattered throughout the keloid tissue. TRPV4-containing cells also stain positively for the fibroblast marker glial fibrillary acidic protein (GFAP) indicating the fibroblasts within keloids express TRPV4. Based on previous observations that TRPV4 stimulates collagen synthesis and deposition, these findings suggest that TRPV4 may be involved in collagen deposition in keloid formation and raise the possibility that pharmacological blockade of TRPV4 may be used to prevent or treat keloid formation.

1. A method for treating a subject suffering from or reducing the likelihood of developing a pancreatic disease or disorder, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

2. The method of claim 1, wherein the Piezo1 antagonist comprises GsMTx-4.

3. The method of claim 1, wherein the TRPV4 antagonist comprises Ruthenium Red, RN-1734, HC-067047, RN-9893, GSK2798745, GSK2193874, or combinations thereof.

4. The method of claim 1, wherein the PLA2 antagonist comprises YM26734, AACOCF₃, or a combination thereof.

5. The method of claim 1, wherein the pancreatic disease or disorder comprises pancreatitis, pancreatic fibrosis, pancreatic cancer, metastatic pancreatic cancer, or combinations thereof.

6. The method of claim 5, wherein the pancreatic cancer or the metastatic pancreatic cancer comprises pancreatic ductal adenocarcinoma (PDAC).

7. The method of claim 1, further comprising administering one or more additional therapeutic agents to the subject.

8. The method of claim 7, wherein the one or more additional therapeutic agents is selected from chemotherapeutic agents, anticancer agents, anti-inflammatory agents, antibiotics, steroids, or combinations thereof.

9. The method of claim 7, wherein the one or more additional therapeutic agents is administered to the subject before administration of the pharmaceutical composition comprising the inhibitory molecule.

10. The method of claim 7, wherein the one or more additional therapeutic agents is administered to the subject concurrently with administration of the pharmaceutical composition comprising the inhibitory molecule.

11. The method of claim 7, wherein the one or more additional therapeutic agents is administered to the subject after administration of the pharmaceutical composition comprising the inhibitory molecule.

12. A method for treating or reducing the likelihood of pancreatic stellate cell activation and/or activation of a fibrinogenic or inflammatory phenotype in a subject, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

13. A method for treating or reducing the likelihood of a subject developing keloids, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

14. The method of claim 13, wherein the Piezo1 antagonist comprises GsMTx-4.

15. The method of claim 13, wherein the TRPV4 antagonist comprises Ruthenium Red, RN-1734, HC-067047, RN-9893, GSK2798745, GSK2193874, or combinations thereof.

16. The method of claim 13, wherein the PLA2 antagonist comprises YM26734, AACOCF₃, or a combination thereof.

17. The method of claim 13, further comprising administering one or more additional therapeutic agents selected from anti-inflammatory agents, antibiotics, steroids, or combinations thereof to the subject.

18. (canceled)

19. The method of claim 17, wherein the one or more additional therapeutic agents is administered to the subject before, concurrently, or after administration of the pharmaceutical composition comprising the inhibitory molecule.

20-21. (canceled)

22. A method for treating or reducing the likelihood of fibroblast activation and/or activation of a fibrinogenic or inflammatory phenotype in a subject, the method comprising: administering to the subject a therapeutically effective amount of a pharmaceutical composition comprising one or more inhibitory molecules comprising a Piezo1 antagonist, a PLA2 antagonist, a TRPV4 antagonist, or combinations thereof.

23. A pharmaceutical composition for treating a subject suffering from, or reducing the likelihood of developing, a pancreatic disease or disorder or keloid, the pharmaceutical composition comprising one or more inhibitory molecules comprising a Piezo1 antagonist, a PLA2 antagonist, a TRPV4 antagonist, or combinations thereof.

24-29. (canceled)
